(19) United States
(12) Patent Application Publication
(10) Pub. No.: US 2024/0335572 A1
(43) Pub. Date: Oct. 10, 2024

(54) RADIOLABELED ANTI-PD-L1 ANTIBODIES FOR IMMUNO-PET IMAGING

(71) Applicant: Regeneron Pharmaceuticals, Inc., Tarrytown, NY (US)

(72) Inventors: Marcus Kelly, New York, NY (US); Dangshe Ma, Millwood, NY (US); William Olson, Yorktown Heights, NY (US); Gavin Thurston, Briarcliff Manor, NY (US)

(21) Appl. No.: 18/750,073

(22) Filed: Jun. 21, 2024

Related U.S. Application Data
(63) Continuation of application No. 17/560,003, filed on Dec. 22, 2021, now Pat. No. 12,053,534.
Application No. 17/560,003 is a continuation of application No. 16/915,894, filed on Jun. 29, 2020, now abandoned.
Application No. 16/915,894 is a division of application No. 15/829,311, filed on Dec. 1, 2017, now Pat. No. 10,736,976.
(60) Provisional application No. 62/428,672, filed on Dec. 1, 2016.
(60) Provisional application No. 62/457,267, filed on Feb. 10, 2017.
(60) Provisional application No. 62/569,773, filed on Oct. 9, 2017.

(51) Int. Cl. A61K 51/10; A61K 51/04; C07K 16/22; C07K 16/28; C07K 19/00

(52) U.S. Cl. CPC A61K 51/1045; A61K 51/0474; A61K 51/1093; C07K 16/22; C07K 16/2827; C07K 19/00; A61K 2121/00; C07K 2317/515; C07K 2317/565

(57) ABSTRACT

Radiolabeled anti-PD-L1 antibodies and their use in immuno-PET imaging are provided herein. Included are methods of detecting the presence of PD-L1 proteins in a patient or sample.

### CROSS-REFERENCE TO RELATED APPLICATIONS

This application is a continuation of U.S. patent application Ser. No. 17/560,003, filed Dec. 22, 2021, which is a continuation application of U.S. patent application Ser. No. 16/915,894, filed Jun. 29, 2020, which is a divisional application of U.S. patent application Ser. No. 15/829,311, filed Dec. 1, 2017, now U.S. Pat. No. 10,736,976 which claims the benefit under 34 U.S.C. § 119(e) of U.S. Provisional Application Ser. No. 62/428,672, filed Dec. 1, 2016, U.S. Provisional Application Ser. No. 62/457,267, filed Feb. 10, 2017, and U.S. Provisional Application Ser. No. 62/569,773, filed Oct. 9, 2017, all of which are herein specifically incorporated by reference in their entireties.

### FIELD

This disclosure relates to radiolabeled anti-PD-L1 antibodies and their use in immuno-PET imaging.

### SEQUENCE LISTING

A copy of the sequence listing is submitted concurrently with the specification electronically via Patent Center. The content of the electronic sequence listing (10305US04_Sequence_Listing_ST26.xml; Size 413,696 bytes; and Date of Creation: Jun. 7, 2024) is herein incorporated by reference in its entirety.

### BACKGROUND

Programmed death-ligand 1 (PD-L1) (also called B7-H1 or CD274) is a 290 amino acid protein receptor ligand expressed widely on both lymphoid and non-lymphoid tissues such as CD4 and CD8 T-cells, macrophage lineage cells, peripheral tissues as well as on tumor cells, and virally-infected cells (Dong et al 1999, Nature Med.). PD-L1 binds to receptors PD-1 and B7-1 which belong to the CD28/CTLA-4 (cytotoxic T lymphocyte antigen)/ICOS (inducible co-stimulator) family of T-cell co-inhibitory receptors (Chen et al 2013, Nature Rev. Immunol. 13: 227-242) and attenuates the immune response by inhibiting T-cell activation. PD-L1 binding to PD-1 or B7-1 results in decreased T-cell proliferation and cytokine secretion, compromising humoral and cellular immune responses in diseases such as cancer, and viral infection. The expression of PD-L1 on tumor cells and virally-infected cells is exploited by tumors and chronic viral infections to evade immune response. PD-L1 is expressed on a wide variety of tumors and studies on animal models have shown that PD-L1 on tumors inhibits T-cell activation and lysis of tumor cells and may lead to increased death of tumor-specific T-cells. In chronic viral infections, PD-L1 expressed on virally-infected cells binds to PD-1 on virus-specific T-cells and these T-cells become “exhausted” with loss of effector functions and proliferative capacity (Freeman 2008, PNAS 105: 10275-10276). The PD-1: PD-L1 system also plays an important role in induced T-regulatory (Treg) cell development and in sustaining Treg function (Francisco et al 2010, Immunol. Rev. 236: 219-242). Blocking PD-L1 with antagonists, including monoclonal antibodies, has been studied in treatments of cancer and chronic viral infections (Ribas 2012, NEJM 366: 2517-2519; Freeman 2008, PNAS 105: 10275-10276; Sheridan 2012, Nature Biotechnology 30: 729-730).

Immuno-positron emission tomography (PET) is a diagnostic imaging tool that utilizes monoclonal antibodies labeled with positron emitters, combining the targeting properties of an antibody with the sensitivity of positron emission tomography cameras. See, e.g., The Oncologist, 12: 1379 (2007); Journal of Nuclear Medicine, 52(8): 1171 (2011). Immuno-PET enables the visualization and quantification of antigen and antibody accumulation in vivo and, as such, can serve as an important tool for diagnostics and complementing therapy. For example, immuno-PET can aid in the selection of potential patient candidates for a particular therapy, as well as in the monitoring of treatment.

As both PD1 and PD-L1 have emerged as targets for immunotherapy, there is need for diagnostic tools for anti-PD1 and/or anti-PD-L1 therapy, including, inter alia, diagnostic tools that enable the detection of suitable patient candidates for said therapy.

### BRIEF SUMMARY

Included in this disclosure are radiolabeled anti-PD-L1 antibody conjugates for use in immuno-PET imaging.

In one aspect, the conjugate comprises an anti-PD-L1 antibody or antigen-binding fragment thereof, a chelating moiety, and a positron emitter.

Provided herein are also processes for synthesizing said conjugates and synthetic intermediates useful for the same.

Provided herein are also methods of imaging a tissue that expresses PD-L1, the methods comprising administering a radiolabeled anti-PD-L1 antibody conjugate described herein to the tissue; and visualizing the PD-L1 expression by positron emission tomography (PET) imaging.

Provided herein are also methods for detecting PD-L1 in a tissue, the methods comprising administering a radiolabeled anti-PD-L1 antibody conjugate described herein to the tissue; and visualizing the PD-L1 expression by PET imaging. In one embodiment, the tissue is present in a human subject. In certain embodiments, the subject is a non-human mammal. In certain embodiments, the subject has a disease or disorder such as cancer, an inflammatory disease, or an infection.

In some aspects, the subject is administered a dose of 5 mg, or 10 mg, or 20 mg, of a radiolabeled anti-PD-L1 antibody conjugate.

Provided herein are also methods for identifying a patient to be suitable for anti-tumor therapy comprising an inhibitor of the PD-1/PD-L1 signaling axis, the methods comprising selecting a patient with a solid tumor, administering a radiolabeled antibody conjugate described herein, and visualizing the administered radiolabeled antibody conjugate in the tumor by PET imaging wherein presence of the radiolabeled antibody conjugate in the tumor identifies the patient as suitable for anti-tumor therapy comprising an inhibitor of the PD-1/PD-L1 signaling axis.

Provided herein are also methods of treating a tumor, the methods comprising selecting a subject with a solid tumor; determining that the solid tumor is PD-L1-positive; and administering an anti-tumor therapy to the subject in need thereof. In certain embodiments, the anti-tumor therapy comprises an inhibitor of the PD-1/PD-L1 signaling axis (e.g., an anti-PD-1 antibody or an anti-PD-L1 antibody). In certain embodiments, the subject is administered a radiolabeled antibody conjugate described herein, and localization of the radiolabeled antibody conjugate is imaged via positron emission tomography (PET) imaging to determine if the tumor is PD-L1-positive.

Provided herein are also methods for monitoring the efficacy of an anti-tumor therapy in a subject, wherein the methods comprise selecting a subject with a solid tumor wherein the subject is being treated with an anti-tumor therapy; administering a radiolabeled conjugate described herein to the subject; imaging the localization of the administered radiolabeled conjugate in the tumor by PET imaging; and determining tumor growth, wherein a decrease from the baseline in uptake of the conjugate or radiolabeled signal indicates tumor regression and efficacy of the anti-tumor therapy. In certain embodiments, the anti-tumor therapy comprises an inhibitor of the PD-1/PD-L1 signaling axis (e.g., an anti-PD-1 antibody).

Provided herein are also methods for predicting response of a patient to an anti-tumor therapy comprising an inhibitor of the PD-1/PD-L1 signaling axis, the methods comprising selecting a patient with a solid tumor; and determining if the tumor is PD-L1-positive, wherein if the tumor is PD-L1-positive it indicates a positive response of the patient to an anti-tumor therapy comprising an inhibitor of the PD-1/PD-L1 signaling axis. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is PD-L1-positive.

### BRIEF DESCRIPTION OF THE FIGURES

FIG. 1A depicts SDS-PAGE and FIG. 1B depicts SEC of un-modified anti-PD-L1 antibody and anti-PD-L1 DFO modified antibody.

FIGS. 2A and 2B depict radio-SEC-HPLC after ⁸⁹Zr radiolabeling for Study 1.

FIG. 3 depicts radio-SEC-HPLC of DFO-conjugate (anti-PD-L1) after ⁸⁹Zr radiolabeling for Study 2.

FIG. 4 depicts radio-SEC-HPLC SEC after ⁸⁹Zr radiolabeling Study 3.

FIG. 5A depicts UV280-SEC-HPLC chromatogram and FIG. 5B depicts_radio-iTLC trace after ⁸⁹Zr radiolabeling for Study 1.

FIGS. 6A, 6B, 6C, and 6D shows hPD-L1 expression by tumor cell lines MC38-cOVA/eGFP-mPD-L1−/−hPD-L1^{Tg} (FIG. 6A), LOX-IMVI (FIG. 6B), MDA-MB-231 (FIG. 6C), and SK-Br-3 (FIG. 6D) in vitro, as described in Example 5 herein.

FIG. 7 shows hPD-L1 expression by MC38-cOVA/eGFP-mPD-L1−/−hPD-L1^{Tg} and LOX-IMVI tumor cells with or without interferon-gamma treatment in vitro in a second experiment, as described in Example 5 herein.

FIGS. 8A, 8B, 8C, 8D, 8E, and 8F depict chromatograms generated by SEC-HPLC analysis using samples from radioimmunoconjugate preparations of ⁸⁹Zr-DFO-anti-PD-L1 antibody conjugate for studies shown in FIG. 8A, FIG. 8B, FIG. 8D, and FIG. 8E, and of isotype control radioimmunoconjugate ⁸⁹Zr-DFO-IgG4^{P} for studies shown in FIG. 8C and FIG. 8F. Chromatograms for absorbance at 280 nm are shown in FIG. 8A-FIG. 8C and radio-chromatograms for intensity of γ-emission are shown in FIG. 8D-FIG. 8F. In FIG. 8A-FIG. 8C, elution of buffer components was also detected. These peaks of salts in the sample buffer (retention time>25 min, asterisk “*”) were excluded from the integration of peak areas. Peaks are labeled to indicate HMW (high molecular weight) immunoconjugate (“1”), monomeric immunoconjugate (“2”), unincorporated ⁸⁹Zr (“3”), and salts in the sample buffer (“*”). Abbreviations: mAU=milli absorbance units; cps=counts per second.

FIG. 9 provides ex vivo biodistribution data for ⁸⁹Zr-DFO-anti-PD-L1 antibody conjugate in PD-1hu/hu-PD-L1hu/hu mice. Sixteen mice (2 groups of 8 animals each) were administered a single IV dose of 50 μCi (1 mg/kg)⁸⁹Zr-DFO-anti-PD-L1 antibody conjugate on day 0 and were sacrificed on day 6 (black columns) or day 10 (gray columns) post dosing. Blood, collected via cardiac puncture, and the indicated harvested tissues were weighed and radioactivity was determined. The percent injected dose per gram (% ID/g) values for individual samples collected on day 6 or 10 were calculated relative to the radioactivity of a dose-standard from injected material (⁸⁹Zr-DFO-anti-PD-L1 antibody conjugate) and the weight of the individual samples. Data are plotted as mean±SD.

### DETAILED DESCRIPTION

### 1. Definitions

Unless defined otherwise herein, all technical and scientific terms used herein have the same meaning as commonly understood by one of ordinary skill in the art to which the disclosed subject matter belongs.

The term “PD-L1” refers to programmed death-ligand 1, also known as CD274 and B7H1. The amino acid sequence of full-length PD-L1 is provided in GenBank as accession number NP_054862.1. The term “PD-L1” also includes protein variants of PD-L1. The term “PD-L1” includes recombinant PD-L1 or a fragment thereof. The term also encompasses PD-L1 or a fragment thereof coupled to, for example, histidine tag, mouse or human Fc, or a signal sequence such as ROR1. For example, the term includes sequences comprising a mouse Fc (mIgG2a) or human Fc (hIgG1) at the C-terminal, coupled to amino acid residues 19-239 of full-length PD-L1 (NP_054862.1). Protein variants comprise a histidine tag at the C-terminal, coupled to amino acid residues 19-239 of NP_054862.1. Unless specified as being from a non-human species, the term “PD-L1” means human PD-L1. PD-L1 is a 290 amino acid protein with extracellular IgV-like and IgC-like domains (amino acids 19-239 of full length PD-L1), a transmembrane domain and an intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, and virally-infected cells and is a component of the immunosuppressive milieu (Ribas 2012, NEJM 366: 2517-2519). PD-L1 binds to one of two T-cell co-inhibitors PD-1 and B7-1.

The term “PD-1” refers to the programmed death-1 protein, a T-cell co-inhibitor, also known as CD279. The amino acid sequence of full-length PD-1 is provided in GenBank as accession number NP_005009.2. The term also encompasses PD-1 or a fragment thereof coupled to, for example, histidine tag, mouse or human Fc, or a signal sequence such as ROR1. For example, the term includes sequences comprising a mouse Fc (mIgG2a) or human Fc (hIgG1) at the C-terminal, coupled to amino acid residues 25-170 of NP_005009.2 with a C93S change. PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-L1 and PD-L2.

The term “B7-1” refers to the T-lymphocyte activation antigen, also known as costimulatory factor CD80. B7-1 is a 288 amino acid membrane receptor with an extracellular N-terminal domain which comprises IgV-like (aa 37-138) and IgC-like (aa 154-232) regions, a transmembrane domain (aa 243-263) and a C-terminal intracellular region (aa 263-288). The amino acid sequence of full-length B7-1 is provided in GenBank as accession number NP_005182.1.

As used herein, the term “T-cell co-inhibitor” refers to a ligand and/or receptor which modulates the immune response via T-cell activation or suppression. The term “T-cell co-inhibitor”, also known as T-cell co-signaling molecule, includes, but is not limited to, PD-1, lymphocyte activation gene 3 protein (LAG-3, also known as CD223), cytotoxic T-lymphocyte antigen-4 (CTLA-4), B and T lymphocyte attenuator (BTLA), CD-28, 2B4, LY108, T-cell immunoglobulin and mucin-3 (TIM3), T-cell immunoreceptor with immunoglobulin and ITIM (TIGIT; also known as VSIG9), leucocyte associated immunoglobulin-like receptor 1 (LAIR1; also known as CD305), inducible T-cell costimulator (ICOS; also known as CD278), B7-1 (CD80), and CD160.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-PD-L1 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes fully human anti-PD-L1 monoclonal antibodies comprising variants of any of the HCVR, LCVR, and/or CDR amino acid sequences disclosed herein having one or more conservative substitutions. For example, the present disclosure includes anti-PD-L1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multi-specific antigen-binding molecules” also includes bi-specific, tri-specific or multi-specific antibodies or antigen-binding fragments thereof. In certain embodiments, an antibody of the present disclosure is functionally linked to another antibody or antigen-binding fragment thereof to produce a bispecific antibody with a second binding specificity. Bispecific and multi-specific antibodies of the present disclosure are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to PD-L1. Moreover, multi-specific antibodies that bind to one domain in PD-L1 and one or more additional antigens or a bi-specific that binds to two different regions of PD-L1 are nonetheless considered antibodies that “specifically bind”, as used herein.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to PD-L1.

An “isolated antibody”, as used herein, is intended to refer to an antibody that is substantially free of other antibodies (Abs) having different antigenic specificities (e.g., an isolated antibody that specifically binds PD-L1, or a fragment thereof, is substantially free of Abs that specifically bind antigens other than PD-L1.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix. Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, in need of amelioration, prevention and/or treatment of a disease or disorder such as chronic viral infection, cancer or autoimmune disease.

### II. Radiolabeled Immunoconjugates of PD-L1 Antibodies for Immuno-PET Imaging

Provided herein are radiolabeled antigen-binding proteins that bind programmed death-ligand 1 (PD-L1). In some embodiments, the radiolabeled antigen-binding proteins comprise an antigen-binding protein covalently linked to one or more chelating moieties, which are chemical moieties that are capable of chelating a positron emitter.

In some embodiments, provided herein are antigen-binding proteins that bind PD-L1, e.g., antibodies, wherein said antigen-binding proteins that bind PD-L1 are covalently bonded to one or more moieties having the following structure:

-L-M_{Z}

wherein L is a chelating moiety; M is a positron emitter; and z, independently at each occurrence, is 0 or 1; and wherein at least one of z is 1.

In some embodiments, the radiolabeled antigen-binding protein is a compound of Formula (I):

M-L-A-[L-M_{Z}]ₖ (I)

A is a protein that binds PD-L1; L is a chelating moiety; M is a positron emitter; z is 0 or 1; and k is an integer from 0-30. In some embodiments, k is 1.

In certain embodiments, the radiolabeled antigen-binding protein is a compound of Formula (II):

A-[L-M]ₖ (II)

wherein A is a protein that binds PD-L1; L is a chelating moiety; M is a positron emitter; and k is an integer from 1-30.

In some embodiments, provided herein are compositions comprising a conjugate having the following structure:

A-Lₖ

wherein A is a protein that binds PD-L1; L is a chelating moiety; and k is an integer from 1-30; wherein the conjugate is chelated with a positron emitter in an amount sufficient to provide a specific activity suitable for clinical PET imaging.

Suitable binding proteins, chelating moieties, and positron emitters are provided below.

### A. PD-L1 Binding Proteins

Suitable PD-L1 binding protein are proteins that specifically bind to PD-L1, including those described in US Patent Publication No. US 2015-0203580 A1, incorporated herein by reference in its entirety. Exemplary anti-PD-L1 antibodies of the present disclosure are listed in Table 1 of US Patent Publication No. US 2015-0203580 A1, also presented below.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H2M8306N | 2 | 4 | 6 | 8 | 10 | 12 | 14 | 16 |
| H2M8307N | 18 | 20 | 22 | 24 | 26 | 28 | 30 | 32 |
| H2M8309N | 34 | 36 | 38 | 40 | 42 | 44 | 46 | 48 |
| H2M8310N | 50 | 52 | 54 | 56 | 58 | 60 | 62 | 64 |
| H2M8312N | 66 | 68 | 70 | 72 | 74 | 76 | 78 | 80 |
| H2M8314N | 82 | 84 | 86 | 88 | 90 | 92 | 94 | 96 |
| H2M8316N | 98 | 100 | 102 | 104 | 106 | 108 | 110 | 112 |
| H2M8317N | 114 | 116 | 118 | 120 | 122 | 124 | 126 | 128 |
| H2M8321N | 130 | 132 | 134 | 136 | 138 | 140 | 142 | 144 |
| H2M8323N | 146 | 148 | 150 | 152 | 154 | 156 | 158 | 160 |
| H2M8718N | 162 | 164 | 166 | 168 | 170 | 172 | 174 | 176 |
| H2M8718N2 | 178 | 180 | 182 | 184 | 170 | 172 | 174 | 176 |
| H2M8719N | 186 | 188 | 190 | 192 | 194 | 196 | 198 | 200 |
| H1H9323P | 202 | 204 | 206 | 208 | 210 | 212 | 214 | 216 |
| H1H9327P | 218 | 220 | 222 | 224 | 226 | 228 | 230 | 232 |
| H1H9329P | 234 | 236 | 238 | 240 | 242 | 244 | 246 | 248 |
| H1H9336P | 250 | 252 | 254 | 256 | 258 | 260 | 262 | 264 |
| H1H9344P2 | 266 | 268 | 270 | 272 | 274 | 276 | 278 | 280 |
| H1H9345P2 | 282 | 284 | 286 | 288 | 274 | 276 | 278 | 280 |
| H1H9351P2 | 290 | 292 | 294 | 296 | 274 | 276 | 278 | 280 |
| H1H9354P2 | 298 | 300 | 302 | 304 | 274 | 276 | 278 | 280 |
| H1H9364P2 | 306 | 308 | 310 | 312 | 274 | 276 | 278 | 280 |
| H1H9373P2 | 314 | 316 | 318 | 320 | 274 | 276 | 278 | 280 |
| H1H9382P2 | 322 | 324 | 326 | 328 | 274 | 276 | 278 | 280 |
| H1H9387P2 | 330 | 332 | 334 | 336 | 274 | 276 | 278 | 280 |
| H1H9396P2 | 338 | 340 | 342 | 344 | 274 | 276 | 278 | 280 |

Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-PD-L1 antibodies.

In some embodiments, the binding protein is an antibody or antigen binding fragment comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

In some embodiments, the binding protein is an antibody or antigen binding fragment comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

In some embodiments, the binding protein is an antibody or antigen binding fragment comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present disclosure provides antibodies, or antigen-binding fragments thereof, comprising an HCVR/LCVR amino acid sequence pair contained within any of the exemplary anti-PD-L1 antibodies listed in Table 1. In certain embodiments, the HCVR/LCVR amino acid sequence pair is selected from the group consisting of SEQ ID NOs: 2/10, 18/26, 34/42, 50/58, 66/74, 82/90, 98/106, 114/122, 130/138, 146/154, 162/170, 178/170, 186/194, 202/210, 218/226, 234/242, 250/258, 266/274, 282/274, 290/274, 298/274, 306/274, 314/274, 322/274, 330/274, and 338/274. In certain embodiments, the HCVR/LCVR amino acid sequence pair is selected from one of SEQ ID NOs: 82/90 (e.g., H2M8314N), 162/170 (e.g., H2M8718N), 306/274 (e.g., H1H9364P2), and 314/274 (e.g., H1H9373P2). In certain other embodiments, the HCVR/LCVR amino acid sequence pair is selected from one of SEQ ID NOs: 98/106 (e.g., H2M8316N), 146/154 (e.g., H2M8323N), 290/274 (e.g., H1H9351P2), and 330/274 (e.g., H1H9387P2).

In some embodiments, the binding protein is an antibody or antigen binding fragment comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

In some embodiments, the binding protein is an antibody or antigen binding fragment comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present disclosure provides antibodies, or antigen-binding fragments thereof, comprising an HCDR3/LCDR3 amino acid sequence pair contained within any of the exemplary anti-PD-L1 antibodies listed in Table 1. In certain embodiments, the HCDR3/LCDR3 amino acid sequence pair is selected from the group consisting of SEQ ID NOs: 88/96 (e.g., H2M8314N), 168/176 (e.g., H2M8718N), 312/280 (e.g., H1H9364P2), and 320/280 (e.g., H1H9373P2). In certain other embodiments, the HCDR3/LCDR3 amino acid sequence pair is selected from the group consisting of SEQ ID NOs: 104/112 (e.g., H2M8316N), 152/160 (e.g., H2M8323N), 296/280 (e.g., H1H9351P2), and 336/280 (e.g., H1H9387P2).

In some embodiments, the binding protein is an antibody or antigen binding fragment comprising a set of six CDRs (i.e., HCDR1-HCDR2-HCDR3-LCDR1-LCDR2-LCDR3) contained within any of the exemplary anti-PD-L1 antibodies listed in Table 1. In certain embodiments, the HCDR1-HCDR2-HCDR3-LCDR1-LCDR2-LCDR3 amino acid sequence set is selected from the group consisting of SEQ ID NOs: 84-86-88-92-94-96 (e.g., H2M8314N); 164-166-168-172-174-176 (e.g., H2M8718N); 308-310-312-276-278-280 (e.g., H1H9364P2); and 316-318-320-276-278-280 (e.g., H1H9373P2). In certain other embodiments, the HCDR1-HCDR2-HCDR3-LCDR1-LCDR2-LCDR3 amino acid sequence set is selected from the group consisting of SEQ ID NOs: 100-102-104-108-110-112 (e.g., H2M8316N); 148-150-152-156-158-160 (e.g., H2M8323N); 292-294-296-276-278-280 (e.g., H1H9351P2); and 332-334-336-276-278-280 (e.g., H1H9387P2).

In some embodiments, the binding protein is an antibody or antigen binding fragment comprising a set of six CDRs (i.e., HCDR1-HCDR2-HCDR3-LCDR1-LCDR2-LCDR3) contained within an HCVR/LCVR amino acid sequence pair as defined by any of the exemplary anti-PD-L1 antibodies listed in Table 1. For example, in some embodiments, the binding protein is an antibody or antigen binding fragment comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 82/90 (e.g., H2M8314N), 98/106 (e.g., H2M8316N), 146/154 (e.g., H2M8323N), 162/170 (e.g., H2M8718N), 290/274 (e.g., H1H9351P2), 306/274 (e.g., H1H9364P2), 314/274 (e.g., H1H9373P2) and 330/274 (e.g., H1H9387P2). Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In some embodiments, binding proteins are antibodies and antigen-binding fragments thereof that compete for specific binding to PD-L1 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

In some embodiments, the binding proteins are isolated antibodies and antigen-binding fragments thereof that block PD-L1 binding to PD-1 or to B7-1. In some embodiments, the antibody or antigen-binding fragment thereof that blocks PD-L1 binding to PD-1 or to B7-1 may bind to the same epitope on PD-L1 as PD-1/B7-1 or may bind to a different epitope on PD-L1 as PD-1/B7-1. In certain embodiments, the antibodies of the disclosure that block PD-L1 binding to PD-1 or to B7-1 comprise the CDRs of an HCVR having an amino acid sequence selected from the group consisting of HCVR sequences listed in Table 1; and the CDRs of a LCVR having an amino acid sequence selected from the group consisting of LCVR sequences listed in Table 1.

In alternate embodiments, the present disclosure provides antibodies and antigen-binding fragments thereof that do not block PD-L1 binding to PD-1 or to B7-1. In certain embodiments, the present disclosure provides isolated antibodies or antigen-binding fragments thereof that bind PD-L1, wherein the antibodies or antigen-binding fragments thereof enhance PD-L1 binding to PD-1 or to B7-1. In some embodiments, the isolated antibodies or antigen-binding fragments thereof that enhance PD-L1 binding to PD-1/B7-1 comprise the CDRs of a HCVR, wherein the HCVR has an amino acid sequence selected from the group consisting of SEQ ID NOs: 18, 66, 114, 130, 202, 218, 266, 282, 298, 322 and 338; and the CDRs of a LCVR, wherein the LCVR has an amino acid sequence selected from the group consisting of SEQ ID NOs: 26, 74, 122, 138, 210, 226, and 274. In some embodiments, the isolated antibodies or antigen-binding fragments thereof comprise an HCVR/LCVR amino acid sequence pair selected from the group consisting of SEQ ID NOs: 18/26 (e.g., H2M8307N), 66/74 (e.g., H2M8312N), 114/122 (e.g., H2M8317N), 130/138 (e.g., H2M8321N), 202/210 (e.g., H1H9323P), 218/226 (e.g., H1H9327P), 266/274 (e.g., H1H9344P2), 282/274 (e.g., H1H9345P2), 298/274 (e.g., H1H9354P2), 322/274 (e.g., H1H9382P2), and 338/274 (e.g., H1H9396P2).

In some embodiments, the binding proteins are antibodies and antigen-binding fragments thereof that bind specifically to PD-L1 from human or other species. In certain embodiments, the antibodies may bind to human PD-L1 and/or to cynomolgus PD-L1.

In some embodiments, the binding proteins are antibodies and antigen-binding fragments thereof that cross-compete for binding to PD-L1 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

In one embodiment, the binding protein is an isolated antibody or antigen-binding fragment that has one or more of the following characteristics: (a) blocks the binding of PD-L1 to PD-1 or to B7-1; (b) binds specifically to human PD-L1 and/or cynomolgus PD-L1; (c) inhibits T-cell proliferation in a mixed lymphocyte reaction (MLR) assay; and (d) increases IL-2 and/or interferon-gamma secretion in a MLR assay.

In some embodiments, the binding protein is an antibody or antigen binding fragment thereof may bind specifically to PD-L1 in an agonist manner, i.e., it enhances or stimulates PD-L1 binding and/or activity; in other embodiments, the antibody can bind specifically to PD-L1 in an antagonist manner, i.e., it blocks PD-L1 from binding to its receptor.

In certain embodiments, the antibodies or antigen-binding fragments are bispecific comprising a first binding specificity to PD-L1 and a second binding specificity for a second target epitope. The second target epitope may be another epitope on PD-L1 or on a different protein such as a T-cell co-inhibitor. In certain embodiments, the target epitope may be on a different cell including e.g., a different T-cell, a B-cell, a tumor cell, an autoimmune tissue cell or a virally infected cell.

In some embodiments, the antibodies and antigen-binding fragments of antibodies bind monomeric PD-L1 (e.g., at 25° C. or at 37° C.) with a K_{D} of less than about 318 pM as measured by surface plasmon resonance, e.g., using the assay format as defined in Example 3 of US Patent Publication No. US 2015-0203580 A1, or substantially similar assay. In certain embodiments, the antibodies or antigen-binding fragments thereof bind monomeric PD-L1 with a K_{D} of less than about 300 pM, less than about 250 pM, less than about 150 pM, less than about 100 pM, or less than about 50 pM, as measured by surface plasmon resonance, e.g., using the assay format as defined in Example 3 of US Patent Publication No. US 2015-0203580 A1, or a substantially similar assay.

In some embodiments, the antibodies and antigen-binding fragments thereof bind dimeric PD-L1 (e.g., at 25° C. or at 37° C.) with a K_{D} of less than about 15 pM as measured by surface plasmon resonance, e.g., using the assay format as defined in Example 3 of US Patent Publication No. US 2015-0203580 A1 or sustainably similar assay. In certain embodiments, the antibodies or antigen-binding fragments thereof bind dimeric PD-L1 with a K_{D} of less than about 12 pM, less than about 10 pM, less than about 8 pM, or less than about 5 pM, as measured by surface plasmon resonance, e.g., using the assay format as defined in Example 3 of US Patent Publication No. US 2015-0203580 A1, or a substantially similar assay.

In some embodiments, the antibodies or antigen-binding fragments thereof bind cynomolgus (Macaca fascicularis) PD-L1 (e.g., at 25° C. or at 37° C.) with a K_{D} of less than about 28 nM as measured by surface plasmon resonance, e.g., using the assay format as defined in Example 3 of US Patent Publication No. US 2015-0203580 A1. In certain embodiments, the antibodies or antigen-binding fragments thereof bind cynomolgus PD-L1 with a K_{D} of less than about 25 nM, less than about 20 nM, less than about 15 nM, less than about 10 nM, or less than about 5 nM, as measured by surface plasmon resonance, e.g., using the assay format as defined in Example 3 of US Patent Publication No. US 2015-0203580 A1, or a substantially similar assay.

In some embodiments, the antibodies and antigen-binding fragments thereof bind PD-L1 with a dissociative half-life (t½) of greater than about 1 minute as measured by surface plasmon resonance at 25° C. or 37° C., e.g., using an assay format as defined in Example 3 of US Patent Publication No. US 2015-0203580 A1, or a substantially similar assay. In certain embodiments, the antibodies or antigen-binding fragments bind PD-L1 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, or greater than about 800 minutes, as measured by surface plasmon resonance at 25° C. or 37° C., e.g., using an assay format as defined in Example 3 of US Patent Publication No. US 2015-0203580 A1 (e.g., mAb-capture or antigen-capture format), or a substantially similar assay.

In some embodiments, the antibodies or antigen-binding fragments thereof block PD-L1 binding to PD-1 with an IC₅₀ of less than about 770 pM as determined using a ELISA-based immunoassay assay, e.g., as shown in Example 4 of US Patent Publication No. US 2015-0203580 A1, or a substantially similar assay. In some embodiments, the antibodies or antigen-binding fragments thereof block PD-L1 binding to B7-1 with an IC₅₀ of less than about 10 nM as determined using a ELISA-based immunoassay assay, e.g., as shown in Example 4 of US Patent Publication No. US 2015-0203580 A1, or a substantially similar assay. In some embodiments, the antibodies and antigen-binding fragments thereof bind to PD-L1 and enhance the binding of PD-L1 to PD-1 or to B7-1.

In some embodiments, the antibodies bind to the extracellular domain of PD-L1 or to a fragment of the domain. In some embodiments, the antibodies bind to more than one domain (cross-reactive antibodies). In certain embodiments, the antibodies of the bind to an epitope located in the extracellular domain comprising amino acid residues 19-239 of NP_054862.1.

In certain embodiments, the antibodies function by blocking or inhibiting the PD-1-binding or the B7-1-binding activity associated with PD-L1 by binding to any other region or fragment of the full length protein. In certain embodiments, the antibodies attenuate or modulate the interaction between PD-L1 and PD-1/B7-1.

In certain embodiments, the antibodies are bi-specific antibodies. The bi-specific antibodies can bind one epitope in one domain and can also bind a second epitope in a different domain of PD-L1. In certain embodiments, the bi-specific antibodies bind two different epitopes in the same domain. In one embodiment, the multi-specific antigen-binding molecule comprises a first antigen-binding specificity wherein the first binding specificity comprises the extracellular domain or fragment thereof of PD-1; and a second antigen-binding specificity to another epitope of PD-L1. In another embodiment, the multi-specific antigen-binding molecule comprises a first antigen-binding specificity wherein the first binding specificity comprises the extracellular domain or fragment thereof of B7-1; and a second antigen-binding specificity to another epitope of PD-L1.

In one embodiment, the antibody or fragment thereof is a fully human monoclonal antibody or antigen-binding fragment thereof that binds to PD-L1, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 186, 202, 218, 234, 250, 258, 266, 274, 282, 290, 298, 306, 314, 322, 330 and 338, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 194, 210, 226, 242, 258, and 274, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 192, 208, 224, 240, 256, 272, 280, 288, 296, 304, 312, 320, 328, 336 and 344, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 200, 216, 232, 248, 264, and 280, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 188, 204, 220, 236, 252, 268, 284, 292, 300, 308, 316, 324, 332, and 340, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 190, 206, 222, 238, 254, 270, 286, 294, 302, 310, 318, 326, 334, and 342, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 196, 212, 228, 244, 260, and 276, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 198, 214, 230, 246, 262, and 278, or a substantially similar sequence thereof having at least 90%, at least 95%, at least 98% or at least 99% sequence identity; (v) is a multi-specific antigen-binding molecule comprising a first binding specificity to PD-L1 and a second binding specificity to an antigen selected from the group consisting of PD-L1, a tumor specific antigen, a virally infected cell antigen, and a T-cell co-inhibitor; (vi) binds to human PD-L1 with a K_{D} of about 4 pM to about 645 nM; (vii) binds to cynomolgus PD-L1 with a K_{D} of about 70 pM to about 400 nM; (viii) blocks or enhances the binding of PD-L1 to PD-1 with an IC50≤770 pM; (ix) blocks or enhances the binding of PD-L1 to B7-1 with an IC50≤10 nM; (x) blocks PD-1-induced T-cell down-regulation and/or rescues T-cell signaling in a T-cell/APC luciferase reporter assay; (xi) stimulates T-cell proliferation and activity in a mixed lymphocyte reaction (MLR) assay; (xii) induces IL-2 and/or IFNγ production in a MLR assay; and (xiii) suppresses tumor growth and increases survival in subjects with cancer.

In one embodiment, the antibody or fragment thereof is a fully human monoclonal antibody or antigen-binding fragment thereof that blocks PD-L1 binding to PD-1 or to B7-1, wherein the antibody or fragment thereof exhibits one or more of the following characteristics: (i) comprises a HCVR having an amino acid sequence selected from the group consisting of SEQ ID NO: 82, 98, 146, 162, 290, 306, 314, and 330, or a substantially similar sequence thereof having at least 90%, at least 95%, at least 98% or at least 99% sequence identity; (ii) comprises a LCVR having an amino acid sequence selected from the group consisting of SEQ ID NO: 90, 106, 154, 170, and 274, or a substantially similar sequence thereof having at least 90%, at least 95%, at least 98% or at least 99% sequence identity; (iii) comprises a HCDR3 domain having an amino acid sequence selected from the group consisting of SEQ ID NO: 88, 104, 152, 168, 296, 312, 320, and 336, or a substantially similar sequence thereof having at least 90%, at least 95%, at least 98% or at least 99% sequence identity; and a LCDR3 domain having an amino acid sequence selected from the group consisting of SEQ ID NO: 96, 112, 160, 176, and 280, or a substantially similar sequence thereof having at least 90%, at least 95%, at least 98% or at least 99% sequence identity; (iv) comprises a HCDR1 domain having an amino acid sequence selected from the group consisting of SEQ ID NO: 84, 100, 148, 164, 292, 308, 316, and 332, or a substantially similar sequence thereof having at least 90%, at least 95%, at least 98% or at least 99% sequence identity; a HCDR2 domain having an amino acid sequence selected from the group consisting of SEQ ID NO: 86, 102, 150, 166, 294, 310, 318, and 334, or a substantially similar sequence thereof having at least 90%, at least 95%, at least 98% or at least 99% sequence identity; a LCDR1 domain having an amino acid sequence selected from the group consisting of SEQ ID NO: 92, 108, 156, 172, and 276, or a substantially similar sequence thereof having at least 90%, at least 95%, at least 98% or at least 99% sequence identity; and a LCDR2 domain having an amino acid sequence selected from the group consisting of SEQ ID NO: 94, 110, 158, 174, and 278, or a substantially similar sequence thereof having at least 90%, at least 95%, at least 98% or at least 99% sequence identity; (v) is a multi-specific antigen-binding molecule comprising a first binding specificity to PD-L1 and a second binding specificity to an antigen selected from the group consisting of a different epitope of PD-L1, a tumor specific antigen, a virally-infected cell antigen, and a T-cell co-inhibitor; (vi) binds to human PD-L1 with a K_{D}≤10⁻¹⁰M; (vii) binds to cynomolgus PD-L1 with a K_{D}≤10⁻⁷M; (viii) blocks the binding of PD-L1 to PD-1 or to B7-1; (ix) blocks PD-1-induced T-cell down-regulation and/or rescues T-cell signaling in a T-cell/APC luciferase reporter assay; (xi) stimulates T-cell proliferation and activity in a mixed lymphocyte reaction (MLR) assay; (xii) induces IL-2 and/or IFNγ production in a MLR assay; and (xiii) suppresses tumor growth and increases survival in subjects with cancer.

In certain embodiments, the anti-PD-L1 antibodies or antigen-binding fragments thereof bind an epitope within any one or more of the regions exemplified in PD-L1, either in natural form, or recombinantly produced, or to a fragment thereof. In some embodiments, the antibodies of the disclosure bind to an extracellular region comprising one or more amino acids selected from the group consisting of amino acid residues 19-239 of PD-L1. In some embodiments, the antibodies of the disclosure bind to a region comprising one or more amino acids selected from the group consisting of amino acid residues 1-221 of cynomolgus PD-L1.

In certain embodiments, the antibodies of the disclosure, as shown in Table 1, interact with at least one amino acid sequence selected from the group consisting of amino acid residues ranging from about position 19 to about position 130 of PD-L1; or amino acid residues ranging from about position 130 to about position 153 of PD-L1; or amino acid residues ranging from about position 153 to about position 210 of PD-L1; or to amino acid residues ranging from about position 210 to about position 239 of PD-L1.

In some embodiments, the anti-PD-L1 antibodies bind to the same epitope, or a portion of the epitope, as any of the specific exemplary antibodies described herein in Table 1, or an antibody having the CDR sequences of any of the exemplary antibodies described in Table 1. Likewise, suitable antibodies also include anti-PD-L1 antibodies that compete for binding to PD-L1 or a PD-L1 fragment with any of the specific exemplary antibodies described herein in Table 1, or an antibody having the CDR sequences of any of the exemplary antibodies described in Table 1. For example, suitable antibodies include anti-PD-L1 antibodies that cross-compete for binding to PD-L1 with one or more antibodies as defined in Example 6 of herein (e.g., H2aM8309N, H1H9329P, H1H9336P, H2aM8314N, H2aM8316N, H2AM8718N, H1H9387P2, H1H9351P2, H1H9364P2, H1H9373P2, and H2aM8306N). The present disclosure also includes anti-PD-L1 antibodies that cross-compete for binding to PD-L1 with one or more antibodies as defined in Example 6 of US Patent Publication No. US 2015-0203580 A1 (e.g., H1H9396P2, H2aM8317N, H2aM8321N, H1H9323P, H1H9382P2, H1H9344P2, H1H9345P2 and H1H9354P2).

The antibodies and antigen-binding fragments described herein specifically bind to PD-L1 and modulate the interaction of PD-L1 with PD-1 or with B7-1. The anti-PD-L1 antibodies may bind to PD-L1 with high affinity or with low affinity. In certain embodiments, the antibodies are blocking antibodies wherein the antibodies bind to PD-L1 and block the interaction of PD-L1 with PD-1 or with B7-1. In some embodiments, the blocking antibodies of the disclosure block the binding of PD-L1 to PD-1 or to B7-1 and/or stimulate or enhance T-cell activation. In some embodiments, the blocking antibodies are useful for stimulating or enhancing the immune response and/or for treating a subject suffering from cancer, or a chronic viral infection. The antibodies when administered to a subject in need thereof may reduce the chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection. In certain embodiments, the anti-PD-L1 antibodies that bind to PD-L1 with a low affinity are used as multi-specific antigen-binding molecules wherein the first binding specificity binds to PD-L1 with a low affinity and the second binding specificity binds to an antigen selected from the group consisting of a different epitope of PD-L1, a T-cell co-inhibitor such as PD-1, a tumor specific antigen and an infected-cell-specific antigen.

In certain embodiments, the antibodies of the present disclosure are agonist antibodies, wherein the antibodies bind to PD-L1 and enhance the interaction of PD-L1 and PD-1/B7-1. In some embodiments, the activating antibodies enhance binding of PD-L1 to PD-1 or to B7-1 and/or inhibit or suppress T-cell activation. The activating antibodies of the present disclosure may be useful for inhibiting the immune response in a subject and/or for treating autoimmune disease.

In certain embodiments, the anti-PD-L1 antibodies are multi-specific antigen-binding molecules, wherein they comprise a first binding specificity to PD-L1 and a second binding specificity to an antigen selected from the group consisting of a different epitope of PD-L1, a T-cell co-inhibitor such as PD-1, a tumor specific antigen and an infected-cell-specific antigen. In certain embodiments, the first binding specificity binds to PD-L1 with low affinity, e.g., with a K_{D} of 10⁻⁸ M, 10⁻⁷ M or more.

Certain anti-PD-L1 antibodies of the present disclosure are able to bind to and neutralize the activity of PD-L1, as determined by in vitro or in vivo assays. The ability of the antibodies of the disclosure to bind to and neutralize the activity of PD-L1 may be measured using any standard method known to those skilled in the art, including binding assays, or activity assays, as described herein.

Non-limiting, exemplary in vitro assays for measuring binding activity are illustrated in Example 3 of US Patent Publication No. US 2015-0203580 A1. In Example 3, the binding affinities and kinetic constants of human anti-PD-L1 antibodies for human PD-L1 and cynomolgus PD-L1 were determined by surface plasmon resonance and the measurements were conducted on a T200 Biacore instrument. In Examples 4 and 5 of US Patent Publication No. US 2015-0203580 A1, blocking assays were used to determine the ability of the anti-PD-L1 antibodies to block PD-L1-binding ability of PD-1 or to B7-1 in vitro. In Example 6 of US Patent Publication No. US 2015-0203580 A1, blocking assays were used to determine cross-competition between different anti-PD-L1 antibodies. Example 7 of US Patent Publication No. US 2015-0203580 A1 describes the binding of the antibodies to cells overexpressing PD-L1. In Example 8 of US 2015-0203580 A1, a luciferase assay was used to determine the ability of anti-PD-L1 antibodies to antagonize PD-1/PD-L1 signaling in T-cells.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to PD-L1. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide or fragment thereof of a multi-specific antigen-binding molecule. In such embodiments, the term “antigen-binding fragment” includes, e.g., an extracellular domain of PD-1 which binds specifically to PD-L1. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

The anti-PD-L1 antibodies and antibody fragments of the present disclosure encompass proteins having amino acid sequences that vary from those of the described antibodies, but that retain the ability to bind PD-L1. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

According to certain embodiments of the present disclosure, anti-PD-L1 antibodies comprise an Fc domain comprising one or more mutations which enhance or diminish antibody binding to the FcRn receptor, e.g., at acidic pH as compared to neutral pH. For example, the present disclosure includes anti-PD-L1 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes anti-PD-L1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). In one embodiment, the present disclosure includes anti-PD-L1 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present disclosure.

The present disclosure also includes anti-PD-L1 antibodies comprising a chimeric heavy chain constant (C_{H}) region, wherein the chimeric C_{H} region comprises segments derived from the C_{H} regions of more than one immunoglobulin isotype. For example, the antibodies of the disclosure may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the disclosure comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Ser. No. 14/170,166, filed Jan. 31, 2014, the disclosure of which is hereby incorporated by reference in its entirety).

### B. Positron Emitters and Chelating Moieties

Suitable positron emitters include, but are not limited to, those that form stable complexes with the chelating moiety and have physical half-lives suitable for immuno-PET imaging purposes. Illustrative positron emitters include, but are not limited to, ⁸⁹Zr, ⁶⁸Ga, ⁶⁴Cu, ⁴⁴Sc, and ⁸⁶Y. Suitable positron emitters also include those that directly bond with the PD-L1 binding protein, including, but not limited to, ⁷⁶Br and ¹²⁴I, and those that are introduced via prosthetic group, e.g., ¹⁸F.

The chelating moieties described herein are chemical moieties that are covalently linked to the PD-L1 binding protein, e.g., anti-PD-L1 antibody and comprise a portion capable of chelating a positron emitter, i.e., capable of reacting with a positron emitter to form a coordinated chelate complex. Suitable moieties include those that allow efficient loading of the particular metal and form metal-chelator complexes that are sufficiently stable in vivo for diagnostic uses, e.g., immuno-PET imaging. Illustrative chelating moieties include those that minimize dissociation of the positron emitter and accumulation in mineral bone, plasma proteins, and/or bone marrow depositing to an extent suitable for diagnostic uses.

Examples of chelating moieties include, but are not limited to, those that form stable complexes with positron emitters ⁸⁹Zr, ⁶⁸Ga, ⁶⁴Cu, ⁴⁴Sc, and/or ⁸⁶Y. Illustrative chelating moieties include, but are not limited to, those described in Nature Protocols, 5(4): 739, 2010; Bioconjugate Chem., 26(12): 2579 (2015); Chem Commun (Camb), 51(12): 2301 (2015); Mol. Pharmaceutics, 12: 2142 (2015); Mol. Imaging Biol., 18: 344 (2015); Eur. J. Nucl. Med. Mol. Imaging, 37:250 (2010); Eur. J. Nucl. Med. Mol. Imaging (2016). doi:10.1007/s00259-016-3499-x; Bioconjugate Chem., 26(12): 2579 (2015); WO 2015/140212A1; and U.S. Pat. No. 5,639,879, incorporated by reference in their entireties.

Illustrative chelating moieties also include, but are not limited to, those that comprise desferrioxamine (DFO), 1,4,7,10-tetraacetic acid (DOTA), diethylenetriaminepentaacetic acid (DTPA), ethylenediaminetetraacetic acid (EDTA), (1,4,7,10-Tetraazacyclododecane-1,4,7,10-tetra(methylene phosphonic) acid (DOTP), 1R, 4R, 7R, 10R)-α′α″α′″-Tetramethyl-1,4,7,10-tetraazacyclododecane-1,4,7,10-tetraacetic acid (DOTMA), 1,4,8,11-Tetraazacyclotetradecane-1,4,8, 11-tetraacetic acid (TETA), H₄octapa, H₆phospa, H₂dedpa, H₅decapa, H₂azapa, HOPO, DO2A, 1,4,7,10-Tetrakis(carbamoylmethyl)-1,4,7,10-tetraazacyclododecane (DOTAM), 1,4,7-triazacyclononane-N,N′,N″-triacetic acid (NOTA), 1,4,7,10-Tetrakis(carbamoylmethyl)-1,4,7,10-tetraazacyclododecane (DOTAM), 1,4,8,11-tetraazabicyclo[6.6.2]hexadecane-4, 11-dicetic acid (CB-TE2A), 1,4,7,10-Tetraazacyclododecane (Cyclen), 1,4,8,11-Tetraazacyclotetradecane (Cyclam), octadentate chelators, hexadentate chelators, phosphonate-based chelators, macrocyclic chelators, chelators comprising macrocyclic terephthalamide ligands, bifunctional chelators, fusarinine C and fusarinine C derivative chelators, triacetylfusarinine C (TAFC), ferrioxamine E (FOXE), ferrioxamine B (FOXB), ferrichrome A (FCHA), and the like.

In some embodiments, the chelating moieties are covalently bonded to the PD-L1 binding protein, e.g., antibody or antigen binding fragment thereof, via a linker moiety, which covalently attaches the chelating portion of the chelating moiety to the binding protein. In some embodiments, these linker moieties are formed from a reaction between a reactive moiety of the PD-L1 binding protein, e.g., cysteine or lysine of an antibody, and reactive moiety that is attached to a chelator, including, for example, a p-isothiocyanatobenyl group and the reactive moieties provided in the conjugation methods below. In addition, such linker moieties optionally comprise chemical groups used for purposes of adjusting polarity, solubility, steric interactions, rigidity, and/or the length between the chelating portion and PD-L1 binding protein.

### C. Preparation of Radiolabeled Anti-PD-L1 Conjugates

The radiolabeled anti-PD-L1 protein conjugates can be prepared by (1) reacting a PD-L1 binding protein, e.g., antibody, with a molecule comprising a positron emitter chelator and a moiety reactive to the desirable conjugation site of the PD-L1 binding protein and (2) loading the desirable positron emitter.

Suitable conjugation sites include, but are not limited to, lysine and cysteine, both of which can be, for example, native or engineered, and can be, for example, present on the heavy or light chain of an antibody. Cysteine conjugation sites include, but are not limited to, those obtained from mutation, insertion, or reduction of antibody disulfide bonds. Methods for making cysteine engineered antibodies include, but are not limited to, those disclosed in WO2011/056983. Site-specific conjugation methods can also be used to direct the conjugation reaction to specific sites of an antibody, achieve desirable stoichiometry, and/or achieve desirable drug-to-antibody (DAR) ratios. Such conjugation methods are known to those of ordinary skill in the art and include, but are not limited to cysteine engineering and enzymatic and chemo-enzymatic methods, including, but not limited to, glutamine conjugation, Q295 conjugation, and transglutaminase-mediated conjugation, as well as those described in J. Clin. Immunol., 36: 100 (2016), incorporated herein by reference in its entirety. Suitable moieties reactive to the desirable conjugation site generally enable efficient and facile coupling of the PD-L1 binding protein, e.g., antibody and positron emitter chelator. Moieties reactive to lysine and cysteine sites include electrophilic groups, which are known to those of ordinary skill. In certain aspects, when the desired conjugation site is lysine, the reactive moiety is an isothiocyanate, e.g., p-isothiocyanatobenyl group or reactive ester. In certain aspects, when the desired conjugation site is cysteine, the reactive moiety is a maleimide.

When the chelator is desferrioxamine (DFO), suitable reactive moieties include, but are not limited to, an isothiocyantatobenzyl group, an n-hydroxysuccinimide ester,2,3,5,6 tetraflurorphenol ester, n-succinimidyl-S-acetylthioacetate, and those described in BioMed Research International, Vol 2014, Article ID 203601, incorporated herein by reference in its entirety. In certain embodiments, the PD-L1 binding protein is an antibody and the molecule comprising a positron emitter chelator and moiety reactive to the conjugation site is p-isothiocyantatobenzyl-desferrioxamine (p-SCN-Bn-DFO):

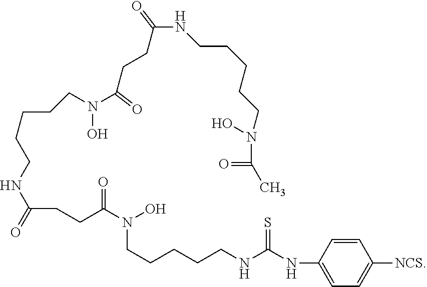

Positron emitter loading is accomplished by incubating the PD-L1 binding protein chelator conjugate with the positron emitter for a time sufficient to allow coordination of said positron emitter to the chelator, e.g., by performing the methods described in the examples provided herein, or substantially similar method.

### D. Illustrative Embodiments of Conjugates

Included in the instant disclosure are radiolabeled antibody conjugates comprising an antibody or antigen binding fragment thereof, that binds human program death ligand 1 (PD-L1), a chelating moiety, and a positron emitter.

In some embodiments, the chelating moiety comprises a chelator capable of forming a complex with ³⁹Zr. In certain embodiments, the chelating moiety comprises desferrioxamine. In certain embodiments, the chelating moiety is p-isothiocyanatobenzyl-desferrioxamine.

In some embodiments, the positron emitter is ³⁹Zr.

In some embodiments, the chelating moiety-to-antibody ratio of the conjugate is from 1 to 2.

In a particular embodiment, chelating moiety is p-isothiocyanatobenzyl-desferrioxamine and the positron emitter is ³⁹Zr. In another particular embodiment, the chelating moiety is p-isothiocyanatobenzyl-desferrioxamine and the positron emitter is ³⁹Zr, and the chelating moiety-to-antibody ratio of the conjugate is from 1 to 2.

In some embodiments, provided herein are antigen-binding proteins that bind PD-L1, wherein said antigen-binding proteins that bind PD-L1 are covalently bonded to one or more moieties having the following structure:

-L-M_{Z}

wherein L is a chelating moiety; M is a positron emitter; and z, independently at each occurrence, is 0 or 1; and wherein at least one of z is 1. In certain embodiments, the radiolabeled antigen-binding protein is a compound of Formula (I):

M-L-A-[L-M_{Z}]ₖ (I)

A is a protein that binds PD-L1; L is a chelating moiety; M is a positron emitter; z is 0 or 1; and k is an integer from 0-30. In some embodiments, k is 1.

In some embodiments, L is:

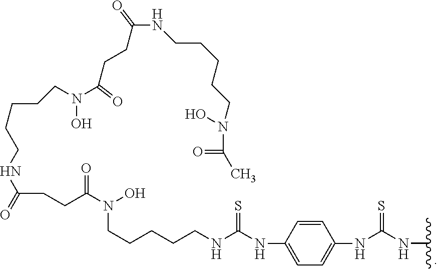 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

In some embodiments, M is ⁸⁹Zr.

In some embodiments, k is an integer from 1 to 2. In some embodiments, k is 1.

In some embodiments, -L-M is

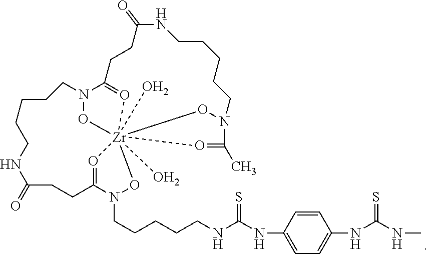

Included in the instant disclosure are also methods of synthesizing a radiolabeled antibody conjugates comprising contacting a compound of Formula (III):

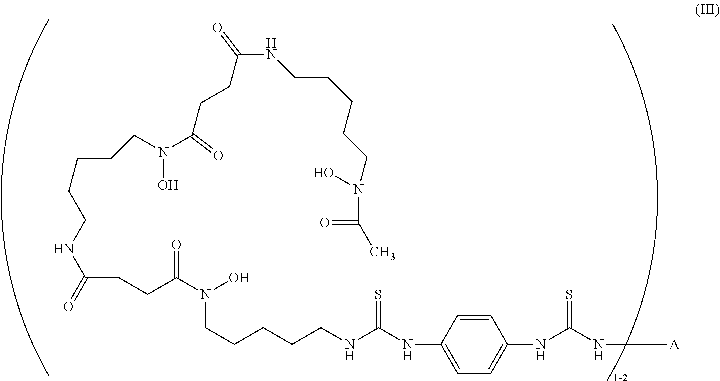

with ³⁹Zr, wherein A is an antibody or antigen-binding fragment thereof that binds PD-L1. In certain embodiments, the compound of Formula (III) is synthesized by contacting an antibody, or antigen binding fragment thereof, that binds PD-L1, with p-SCN-Bn-DFO.

Provided herein is also the product of the reaction between a compound of Formula (III) with ⁸⁹Zr.

Provided herein are compounds of Formula (III):

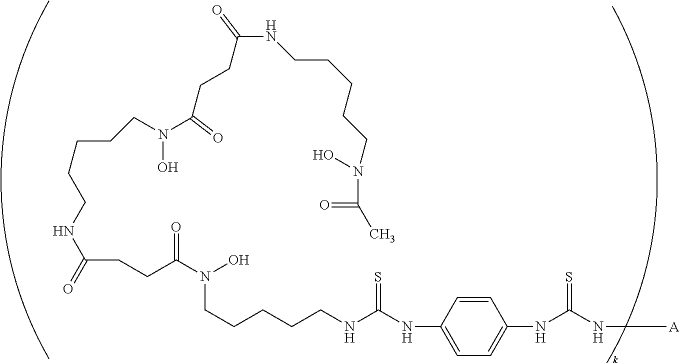

wherein A is an antibody or antigen binding fragment thereof that binds PD-L1 and k is an integer from 1-30. In some embodiments, k is 1 or 2.

In some embodiments, provided herein are compositions comprising a conjugate having the following structure:

A-Lₖ

wherein A is a protein that binds PD-L1; L is a chelating moiety; and k is an integer from 1-30; wherein the conjugate is chelated with a positron emitter in an amount sufficient to provide a specific activity suitable for clinical PET imaging. In some embodiments, the amount of chelated positron emitter is an amount sufficient to provide a specific activity of 1-3 mCi per 1-50 mg of the protein that binds PD-L1.

In some embodiments, the antibody or antigen-binding fragment thereof binds monomeric human programmed death-ligand 1 (PD-L1) with a binding dissociation equilibrium constant (K_{D}) of less than about 310 pM as measured in a surface plasmon resonance assay at 37° C.

In some embodiments, the antibody or antigen-binding fragment thereof binds monomeric human PD-L1 with a K_{D} less than about 180 pM in a surface plasmon resonance assay at 25° C.

In some embodiments, the antibody or antigen-binding fragment thereof binds dimeric human PD-L1 with a K_{D} of less than about 15 pM as measured in a surface plasmon resonance assay at 37° C.

In some embodiments, the antibody or antigen-binding fragment thereof that binds dimeric human PD-L1 with a K_{D} less than about 8 pM in a surface plasmon resonance assay at 25° C.

In some embodiments, the antibody or antigen-binding fragment thereof competes for binding to human PD-L1 with a reference antibody comprising the complementarity determining regions (CDRs) of a HCVR, wherein the HCVR has an amino acid sequence selected from the group consisting of HCVR sequences listed in Table 1; and the CDRs of a LCVR, wherein the LCVR has an amino acid sequence selected from the group consisting of LCVR sequences listed in Table 1. In some embodiments, the reference antibody or antigen-binding fragment thereof comprises an HCVR/LCVR amino acid sequence pair as set forth in Table 1. In some embodiments, the reference antibody comprises an HCVR/LCVR amino acid sequence pair selected from the group consisting of SEQ ID NOs: 82/90, 98/106, 146/154, 162/170, 290/274, 306/274, 314/274 and 330/274.

In some embodiments, the antibody or antigen-binding fragment thereof enhances PD-L1 binding to one of PD-1 or B7-1. In some embodiments, the antibody or antigen binding fragment thereof blocks PD-L1 binding to PD-1 and/or B7-1. In some embodiments, the antibody or antigen binding fragment thereof do not increase or decrease PD-L1 binding to its ligands.

In some embodiments, the antibody or antigen-binding fragment thereof comprises the complementarity determining regions (CDRs) of a HCVR, wherein the HCVR has an amino acid sequence selected from the group consisting of SEQ ID NOs: 18, 66, 114, 130, 202, 218, 266, 282, 298, 322, and 338; and the CDRs of a LCVR, wherein the LCVR has an amino acid sequence selected from the group consisting of SEQ ID NOs: 26, 74, 122, 138, 210, 226, and 274. In certain embodiments, the isolated antibody comprises an HCVR/LCVR amino acid sequence pair selected from the group consisting of SEQ ID NOs: 18/26, 66/74, 114/122, 130/138, 202/210, 218/226, 266/274, 282/274, 298/274, 322/274, and 338/274.

In some embodiments, the antibody is a human monoclonal antibody or antigen-binding fragment thereof that binds specifically to human PD-L1, wherein the antibody or antigen-binding fragment thereof comprises a heavy chain variable region (HCVR) having an amino acid sequence selected from the group consisting of HCVR sequences listed in Table 1.

In some embodiments, the antibody is a human monoclonal antibody or antigen-binding fragment thereof that binds specifically to human PD-L1, wherein the antibody or antigen-binding fragment thereof comprises a light chain variable region (LCVR) having an amino acid sequence selected from the group consisting of LCVR sequences listed in Table 1.

In some embodiments, the antibody a human monoclonal antibody or antigen-binding fragment thereof that binds specifically to human PD-L1, wherein the antibody or antigen-binding fragment thereof comprises (a) a HCVR having an amino acid sequence selected from the group consisting of HCVR sequences listed in Table 1; and (b) a LCVR having an amino acid sequence selected from the group consisting of LCVR sequences listed in Table 1.

In some embodiments, the antibody or antigen-binding fragment thereof comprises three heavy chain complementarity determining regions (CDRs) (HCDR1, HCDR2 and HCDR3) contained within any one of the heavy chain variable region (HCVR) sequences listed in Table 1; and three light chain CDRs (LCDR1, LCDR2 and LCDR3) contained within any one of the light chain variable region (LCVR) sequences listed in Table 1.

In some embodiments, the antibody or antigen-binding fragment thereof comprises:
(a) a HCDR1 domain having an amino acid sequence selected from the group consisting of SEQ ID NOs: 4, 20, 36, 52, 68, 84, 100, 116, 132, 148, 164, 180, 188, 204, 220, 236, 252, 268, 284, 292, 300, 308, 316, 324, 332, and 340;
(b) a HCDR2 domain having an amino acid sequence selected from the group consisting of SEQ ID NOs: 6, 22, 38, 54, 70, 86, 102, 118, 134, 150, 166, 182, 190, 206, 222, 238, 254, 270, 286, 294, 302, 310, 318, 326, 334, and 342;
(c) a HCDR3 domain having an amino acid sequence selected from the group consisting of SEQ ID NOs: 8, 24, 40, 56, 72, 88, 104, 120, 136, 152, 168, 184, 192, 208, 224, 240, 256, 272, 288, 296, 304, 312, 320, 328, 336, and 344;
(d) a LCDR1 domain having an amino acid sequence selected from the group consisting of SEQ ID NOs: 12, 28, 44, 60, 76, 92, 108, 124, 140, 156, 172, 196, 212, 228, 244, 260, and 276;
(e) a LCDR2 domain having an amino acid sequence selected from the group consisting of SEQ ID NOs: 14, 30, 46, 62, 78, 94, 110, 126, 142, 158, 174, 198, 214, 230, 246, 262, and 278; and
(f) a LCDR3 domain having an amino acid sequence selected from the group consisting of SEQ ID NOs: 16, 32, 48, 64, 80, 96, 112, 128, 144, 160, 176, 200, 216, 232, 248, 264, and 280.

In some embodiments, the antibody or antigen-binding fragment comprises a HCVR/LCVR amino acid sequence pair selected from the group consisting of SEQ ID NOs: 82/90, 98/106, 146/154, 162/170, 290/274, 306/274, 314/274 and 330/274.

In some embodiments, the antibody or antigen-binding fragment thereof comprises the CDRs of a HCVR, wherein the HCVR has an amino acid sequence selected from the group consisting of SEQ ID NOs: 2, 34, 50, 82, 98, 146, 162, 178, 186, 234, 250, 290, 306, 314, and 330; and the CDRs of a LCVR, wherein the LCVR has an amino acid sequence selected from the group consisting of SEQ ID NOs: 10, 42, 58, 90, 106, 154, 170, 194, 242, 258, and 274.

### E. Scaled Manufacturing for Production of Anti-PD-L1 Antibody-Chelator Conjugates

Included in the present disclosure are scaled-up manufacturing processes for producing anti-PD-L1 antibodies conjugated to a chelator. The anti-PD-L1 antibody-chelator conjugates are in a form suitable for radiolabeling.

Good manufacturing processes are adhered to in all aspects of production, including maintaining a sterile environment, practicing aseptic procedures, keeping records of all processes, and documenting product quality, purity, strength, and identity, and any deviations therefrom.

The scaled-up manufacturing process is, in some embodiments, much faster than the manufacturing process for research and development. In some embodiments, the scaled-up manufacturing process can take less than 12 hours, or less than 10 hours, or less than 8 hours, or less than 6 hours, or less than 4 hours, or less than or about 2 hours.

In some embodiments, a first step comprises ultrafiltration and diafiltration (UFDF), using a 30-50 kDa membrane, of the anti-PD-L1 antibody to remove excipients, conjugation interfering species, and salts that inhibit the conjugation process. Exemplary membrane polymers include polyethersulfone (PES), cellulose acetate (CA), and regenerated cellulose (RC). In this step, the antibody is buffer exchanged in a low ionic strength and non-interfering buffer solution. The buffer pH can be between about 4.5 to about 6, or about 5 to about 6, or about 5.3 to about 5.7, or about 5.5. Buffer systems contemplated as useful herein include any buffer system lacking a primary amine. Exemplary buffers include acetate, phosphate, or citrate buffers. The buffer provides protein stability during pre-conjugation processing. The process volume can be further reduced to concentrate the antibody, then sterile filtered.

Following the pre-conjugation UFDF, the concentrated and filtered antibody can be transferred into an amine free carbonate buffer system. The carbonate buffer system can have a pH in a range from about 8.5 to about 9.6, or from about 9.0 to about 9.6, or from about 9.2 to about 9.4, or from about 9.4 to about 9.6, or a pH of about 9.4.

A chelator, for example, DFO, in solvent is added to a target concentration into the buffer system containing the antibody, and additional solvent can be added to the solution to a desired percentage. The chelator can be added in molar excess of the antibody, for example, 3.5-5:1 chelator to antibody. The total reaction volume can be up to 5 L.

The reaction temperature and the reaction time are inversely related. For example, if the reaction temperature is higher, the reaction time is lower. If the reaction temperature is lower, the reaction time is higher. Illustratively, at a temperature above about 18° C., the reaction may take less than 2 hours; at a temperature below 18° C., the reaction may take more than 2 hours.

The conjugation reaction can be terminated by quenching, for example, by the addition of acetic acid.

In some embodiments, conjugation of the antibody with deferoxamine is performed to produce DFO-mAb conjugates. In some embodiments, conjugation of the antibody with p-SCN-Bn-deferoxamine is performed to produce DFO-mAb conjugates.

Exemplary solvents for the chelator include DMSO and DMA. Subsequent UFDF steps utilize membranes, and the membrane is chosen based on the solvent system used in the conjugation step. For example, DMA dissolves PES membranes, so the two could not be used in the same system.

Carbonate buffers are not preferred for stability of the conjugate during long term storage. Thus, once the antibody-chelator conjugates have been formed, they can be buffer exchanged into a buffer chosen specifically for long term storage and stability. Exemplary buffers include citrate, acetate, phosphate, arginine, and histidine buffers. A further UFDF step can be performed to remove residual salts and to provide a suitable concentration, excipient level, and pH of the conjugated monoclonal antibody. The resulting antibody-chelator conjugates can be sterile filtered and stored for subsequent formulation.

### III. Methods of Using Radiolabeled Immunoconjugates

In certain aspects, the present disclosure provides diagnostic and therapeutic methods of use of the radiolabeled antibody conjugates of the present disclosure.

According to one aspect, the present disclosure provides methods of detecting PD-L1 in a tissue, the methods comprising administering a radiolabeled antibody conjugate of the provided herein to the tissue; and visualizing the PD-L1 expression by positron emission tomography (PET) imaging. In certain embodiments, the tissue comprises cells or cell lines. In certain embodiments, the tissue is present in a subject, wherein the subject is a mammal. In certain embodiments, the subject is a human subject. In certain embodiments, the subject has a disease or disorder selected from the group consisting of cancer, infectious disease and inflammatory disease. In one embodiment, the subject has cancer. In certain embodiments, the infectious disease is bacterial or viral infection caused by, for example, hepatitis B virus (HBV), hepatitis C virus (HCV), human immunodeficiency virus (HIV), and Mycobacterium tuberculosis.

According to one aspect, the present disclosure provides methods of imaging a tissue that expresses PD-L1 comprising administering a radiolabeled antibody conjugate of the present disclosure to the tissue; and visualizing the PD-L1 expression by positron emission tomography (PET) imaging. In one embodiment, the tissue is comprised in a tumor. In one embodiment, the tissue is comprised in a tumor cell culture or tumor cell line. In one embodiment, the tissue is comprised in a tumor lesion in a subject.

According to one aspect, the present disclosure provides methods for measuring response to a therapy, wherein the response to a therapy is measured by measuring inflammation. The methods, according to this aspect, comprise administering a radiolabeled antibody conjugate provided herein to a subject in need thereof and visualizing the PD-L1 expression by positron emission tomography (PET) imaging. In certain embodiments, the inflammation is present in a tumor in the subject. In certain embodiments, an increase in PD-L1 expression correlates to increase in inflammation in the tumor.

According to one aspect, the present disclosure provides methods for determining if a patient is suitable for anti-tumor therapy comprising an inhibitor of the PD-1/PD-L1 signaling axis, the methods comprising selecting a patient with a solid tumor, administering a radiolabeled antibody conjugate of the present disclosure, and localizing the administered radiolabeled antibody conjugate in the tumor by PET imaging wherein presence of the radiolabeled antibody conjugate in the tumor identifies the patient as suitable for anti-tumor therapy comprising an inhibitor of the PD-1/PD-L1 signaling axis.

According to one aspect, the present disclosure provides methods for identifying a candidate for anti-tumor therapy comprising an inhibitor of the PD-1/PD-L1 signaling axis, the methods comprising selecting a patient with a solid tumor, administering a radiolabeled antibody conjugate of the present disclosure, and localizing the administered radiolabeled antibody conjugate in the tumor by PET imaging wherein presence of the radiolabeled antibody conjugate in the tumor identifies the patient as suitable for anti-tumor therapy comprising an inhibitor of the PD-1/PD-L1 signaling axis.

According to one aspect, the present disclosure provides methods for predicting response of a patient to an anti-tumor therapy comprising an inhibitor of the PD-1/PD-L1 signaling axis, the methods comprising selecting a patient with a solid tumor, determining if the tumor is PD-L1-positive, wherein a positive response of the patient is predicted if the tumor is PD-L1-positive. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is PD-L1-positive.

According to one aspect, the present disclosure provides methods for detecting a PD-L1-positive tumor in a subject. The methods, according to this aspect, comprise selecting a subject with a solid tumor; administering a radiolabeled antibody conjugate of the present disclosure to the subject; and determining localization of the radiolabeled antibody conjugate by PET imaging, wherein presence of the radiolabeled antibody conjugate in a tumor indicates that the tumor is PD-L1-positive.

As used herein, the expression “a subject in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient”. For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression includes subjects with primary or established tumors. In specific embodiments, the expression includes human subjects that have and/or need treatment for a solid tumor, e.g., colon cancer, breast cancer, lung cancer, prostate cancer, skin cancer, liver cancer, bone cancer, ovarian cancer, cervical cancer, pancreatic cancer, head and neck cancer, and brain cancer. The term includes subjects with primary or metastatic tumors (advanced malignancies). In certain embodiments, the expression “a subject in need thereof” includes patients with a solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel). In certain embodiments, the expression “a subject in need thereof” includes patients with a solid tumor which has been treated with one or more lines of prior therapy but which has subsequently relapsed or metastasized. In certain embodiments, the term includes subjects having an inflammatory disease or disorder including, but not limited to, cancer, rheumatoid arthritis, atherosclerosis, periodontitis, hay fever, heart disease, coronary artery disease, infectious disease, bronchitis, dermatitis, meningitis, asthma, tuberculosis, ulcerative colitis, Crohn's disease, inflammatory bowel disease, hepatitis, sinusitis, psoriasis, allergy, fibrosis, lupus, vasiculitis, ankylosing spondylitis, Graves' disease, Celiac disease, fibromyalgia, and transplant rejection.

In certain embodiments, the methods of the present disclosure are used in a subject with a solid tumor. The terms “tumor”, “cancer” and “malignancy” are interchangeably used herein. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas. In certain embodiments, the term “solid tumor” includes cancers including, but not limited to, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophageal cancer, head and neck cancer, salivary gland cancer, and myeloma.

According to one aspect, the present disclosure provides methods of treating a tumor in a subject. The methods, according to this aspect, comprise selecting a subject with a solid tumor; determining that the tumor is PD-L1-positive; and administering one or more doses of an inhibitor of the PD-1/PD-L1 signaling axis. In certain embodiments, the tumor is determined to be PD-L1-positive by administering a radiolabeled antibody conjugate of the present disclosure to the subject; and visualizing the radiolabeled antibody conjugate in the tumor by PET imaging, wherein presence of the radiolabeled antibody conjugate in the tumor indicates that the tumor is PD-L1-positive.

As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, and/or to increase duration of survival of the subject.

According to one aspect, the present disclosure provides methods for monitoring the efficacy of an anti-tumor therapy in a subject, wherein the methods comprise selecting a subject with a solid tumor wherein the subject is being treated with an anti-tumor therapy; administering a radiolabeled antibody conjugate of the present disclosure to the subject; imaging the localization of the administered radiolabeled conjugate in the tumor by PET imaging; and determining tumor growth, wherein a decrease from the baseline in radiolabeled signal indicates tumor regression and efficacy of the anti-tumor therapy. In certain embodiments, the anti-tumor therapy comprises an inhibitor of the PD-1/PD-L1 signaling axis (e.g., an anti-PD-1 antibody).

In certain embodiments, the present disclosure provides methods to assess changes in the inflammatory state of a tumor, the methods comprising selecting a subject with a solid tumor wherein the subject is being treated with an anti-tumor therapy; administering a radiolabeled antibody conjugate provided herein to the subject; and imaging the localization of the administered radiolabeled conjugate in the tumor by PET imaging, wherein an increase from the baseline in radiolabeled signal indicates increase in inflammation and efficacy of the anti-tumor therapy. In certain embodiments, the anti-tumor therapy comprises an inhibitor of the PD-1/PD-L1 signaling axis (e.g., an anti-PD-1 antibody).

As used herein, the term “baseline,” with respect to the PD-L1 expression in the tumor, means the numerical value of uptake of the radiolabeled conjugate for a subject prior to or at the time of administration of a dose of anti-tumor therapy. The uptake of the radiolabeled conjugate is determined using methods known in the art (see, for example, Oosting et al 2015, J. Nucl. Med. 56: 63-69). In certain embodiments, the anti-tumor therapy comprises an inhibitor of the PD-1/PD-L1 signaling axis.

To determine whether there is tumor regression, the uptake of the radiolabeled conjugate is quantified at baseline and at one or more time points after administration of the inhibitor of the PD-1/PD-L1 signaling axis (e.g., an anti-PD-1 antibody). For example, the uptake of the administered radiolabeled antibody conjugate (e.g., radiolabeled anti-PD-L1 antibody conjugate) may be measured at day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the inhibitor of the PD-1/PD-L1 signaling axis (e.g., an anti-PD-1 antibody). The difference between the value of the uptake at a particular time point following initiation of treatment and the value of the uptake at baseline is used to establish whether there has been a difference in amount of tumor tissue (tumor regression or progression). For example, a decrease from baseline in the uptake upon treatment with at least one dose of the inhibitor of the PD-1/PD-L1 signaling axis means tumor regression and indicates efficacy of the anti-tumor therapy.

In certain embodiments, the radiolabeled antibody conjugate is administered intravenously or subcutaneously to the subject. In certain embodiments, the radiolabeled antibody conjugate is administered intra-tumorally. Upon administration, the radiolabeled antibody conjugate is localized in the tumor. The localized radiolabeled antibody conjugate is imaged by PET imaging and the uptake of the radiolabeled antibody conjugate by the tumor is measured by methods known in the art. In certain embodiments, the imaging is carried out 1, 2, 3, 4, 5, 6 or 7 days after administration of the radiolabeled conjugate. In certain embodiments, the imaging is carried out on the same day upon administration of the radiolabeled antibody conjugate.

In certain embodiments, the antibody or antigen-binding fragment thereof that binds specifically to PD-L1. In certain embodiments, the anti-PD-L1 antibody comprises the CDRs of a HCVR, wherein the HCVR has an amino acid sequence selected from the group consisting of SEQ ID NOs: 2, 34, 50, 82, 98, 146, 162, 178, 186, 234, 250, 290, 306, 314, and 330; and the CDRs of a LCVR, wherein the LCVR has an amino acid sequence selected from the group consisting of SEQ ID NOs: 10, 42, 58, 90, 106, 154, 170, 194, 242, 258, and 274.

In certain embodiments, the inhibitor of the PD-1/PD-L1 signaling axis comprises an antibody or antigen-binding fragment thereof that binds specifically to PD-1. In certain embodiments, the anti-PD-1 antibody is selected from the group consisting of nivolumab, pembrolizumab and REGN2810. In certain other embodiments, the inhibitor of the PD-1/PD-L1 signaling axis comprises an antibody or antigen-binding fragment thereof that binds specifically to PD-L1. In one embodiment, the anti-PD-L1 antibody is atezolizumab. In one embodiment, the anti-PD-L1 antibody comprises an HCVR of SEQ ID NO: 82 and a LCVR of SEQ ID NO: 90.

### IV. Examples

Certain embodiments of the disclosure are illustrated by the following non-limiting examples.

### Example 1: Generation of Human Antibodies to PD-L1

Human anti PD-L1 antibodies, including those listed in Table 1, were prepared and characterized as described in US Patent Publication No. US 2015-0203580 A1, which is incorporated herein by reference in its entirety. In brief, human antibodies to PD-L1 were generated using a fragment of PD-L1 that ranges from about amino acids 19-239 of PD-L1 (Genbank Accession No. NP_054862.1). The immunogen was administered directly, with an adjuvant to stimulate the immune response, to a VELOCIMMUNE® mouse comprising DNA encoding human Immunoglobulin heavy and kappa light chain variable regions. The antibody immune response was monitored by a PD-L1-specific immunoassay. When a desired immune response was achieved splenocytes were harvested and fused with mouse myeloma cells to preserve their viability and form hybridoma cell lines. The hybridoma cell lines were screened and selected to identify cell lines that produce PD-L1-specific antibodies. Using this technique, and the immunogen described above, several anti-PD-L1 chimeric antibodies (i.e., antibodies possessing human variable domains and mouse constant domains) were obtained; exemplary antibodies generated in this manner were designated as H2M8306N, H2M8307N, H2M8309N, H2M8310N, H2M8312N, H2M8314N, H2M8316N, H2M8317N, H2M8321N, H2M8323N, H2M8718N, H2M8718N2, and H2M8719N.

Anti-PD-L1 antibodies were also isolated directly from antigen-positive B cells without fusion to myeloma cells, as described in U.S. 2007/0280945A1, herein specifically incorporated by reference in its entirety. Using this method, several fully human anti-PD-L1 antibodies (i.e., antibodies possessing human variable domains and human constant domains) were obtained; exemplary antibodies generated in this manner were designated as follows: H1H9323P, H1H9327P, H1H9329P, H1H9336P, H1H9344P2, H1H9345P2, H1H9351P2, H1H9354P2, H1H9364P2, H1H9373P2, H1H9382P2, H1H9387P2, and H1H9396P2.

### Example 2: Conjugation of Anti-PD-L1 Antibody H4H8314N with p-SCN-Bn-DFO

In order to modify the parental anti-PD-L1 antibody, H4H8314N, and an isotype control antibody to be suitable for ImmunoPET studies with radiolabeling, a chelator, p-SCN-bn-Deferoxamine (DFO; Macrocylics, Cat #: B-705), was attached to the antibodies.

For the modification, H4H8314N was first buffer exchanged into PBS, pH 7.2 from histidine buffer by dialysis at 4° C. overnight (Slide-A-Lyzer Dialysis Cassette G2 10 k MWCO; ThermoScientific) then buffer exchanged again using a PD-10 column (GE Healthcare, Cat. #: 17-0851-01) into a buffer composed of 50 mM carbonate buffer, 150 mM NaCl, pH 9.0 (conjugation buffer). To determine the concentration following the buffer exchanges, the samples were measured on a Nanodrop 2000 UV/VIS spectrometer (Thermo Scientific) using the MacVector sequence based extinction coefficient of 1.46 g/L (see Table 2). In 15 a mL polypropylene tube, 773.9 uL of H4H8314N (12.5 mg) was added to 1676.1 uL of conjugation buffer. In a separate vial, 29.3 uL of DMSO was added to 20.7 uL of DFO. In one-quarter increments, this DFO solution was added to the H1H8314N solution, each time gently being mixed by pipetting up-and-down. The final solution was 5 mg/mL H4H8314N in conjugation buffer, 2% DMSO with 6-fold mole-to-mole excess of DFO. This solution was allowed to incubate in a 37° C. water bath with no additional stirring.

After 30 minutes at 37° C., the solution was promptly passed through a PD-10 desalting column (GE Healthcare, Cat. #: 17-0851-01), pre-equilibrated with a buffer containing 250 mM NaAcO at pH 5.4 (formulation buffer). The final solution was sterile-filtered via a syringe filter (Acrodisc 13 mm syringe filter, Pall Corporation, Cat #: 4602). The concentration and DFO-to-Antibody Ratio (DAR) was subsequently measured by UV/VIS spectroscopy. For the absorbance measurement, the DFO-conjugated antibody was measured against the formulation buffer at 252 nm (A252), 280 nm (A280) and 600 nm (A600). For the calculation, the background was corrected at each absorbance value using the equation:

$${A}_{λ}^{′} = {A}_{λ} - {A}_{600}$$

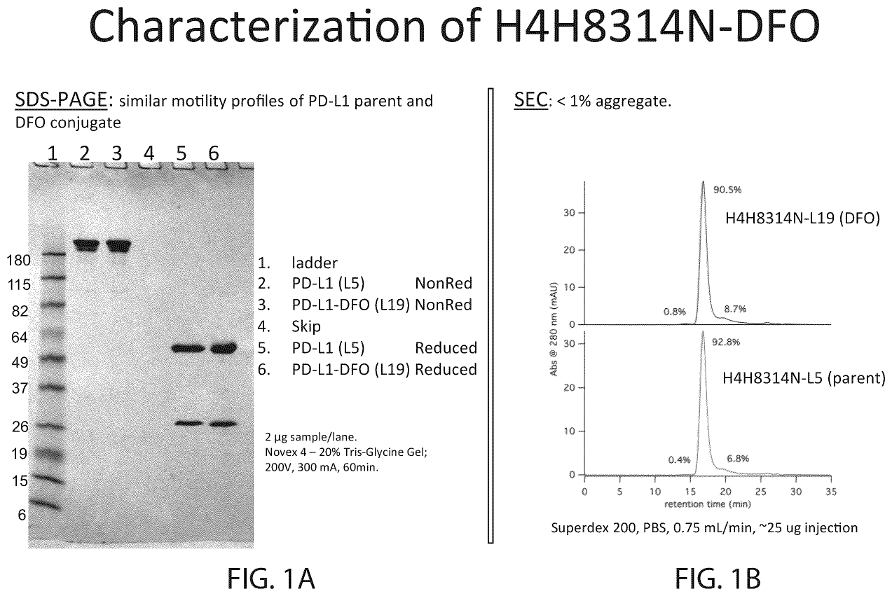

The antibody conjugate was tested for aggregation using SEC chromatography, with 25 ug of the sample injected onto a Superdex 200 column (GE Healthcare, Cat. No. 17-5175-01) monitored at 280 nm with a PBS mobile phase (0.75 mL/min). The antibody integrity was evaluated by SDS-PAGE 4-20% Tris/Gly pre-cast gel (Novex) with 2 ug of the sample loaded. The gel is shown in FIG. 1. The antibody concentration, conjugate concentration, and DAR were calculated using the equations below:

### Antibody Concentration Calculation

$$Conc mAb ( mg / mL ) = \frac{{A}_{280}^{′}}{{ϵ}_{280}}$$

### Conjugate Concentration Calculation

$$Conc conjugate ( mg / mL ) = \frac{{A}_{252}^{′} - 1.53 {A}_{280}^{′}}{{ϵ}_{252} - 1.53 {ϵ}_{280}}$$

### DAR Calculation

$$DAR = \frac{{ϵ}_{252} {A}_{280}^{′} - {ϵ}_{280} {A}_{252}^{′}}{18800 {A}_{252}^{′} - 28700 {A}_{280}^{′}}$$

TABLE 2
Molar extinction coefficients and molecular weight

| | MW | ε280 | ε252 |
|---|---|---|---|
| Antibody | (g mol⁻¹) | (L g⁻¹cm⁻¹) | (L g⁻¹cm⁻¹) |
| H4H8314N | 144984 | 1.46 | 0.553 |

TABLE 3
UV DAR, percent aggregate and concentration post DFO-attachment

| | | Concentration | |
|---|---|---|---|
| Antibody | UV DAR | (mg/mL) | % aggregate |
| H4H8314N | 1.2 | 3.34 | <1% |

### Example 3: ⁸⁹Zr Chelation of DFO Conjugated Monoclonal Antibodies

For use in ImmunoPET in vivo studies, the DFO-conjugated anti-PD-L1 antibody, H4H8314N, and a DFO-conjugated isotype control antibody were radiolabeled with ⁸⁹Zr.

DFO-conjugated antibody (250 or 750 ug) was first brought to 1.25 mg/mL in 1 M HEPES, pH 7.2. The recipe of DFO-Ab conjugate solution for each study is listed in Table 4. Separately, ⁸⁹Zr solution was prepared using the recipe for each corresponding study shown in Table 5. Stock ⁸⁹Zr-oxalic acid solution was obtained from PerkinElmer or 3D Imaging. If the radioactivity concentration of the stock solution was low (see Table 5), a neutralization step was performed with 1 M borate, pH 9.0. The final radioactivity of the solution was first confirmed using a Capintec CRC-25R dose calibrator (Capintec #520), then immediately combined with the DFO-Ab conjugate solution, gently mixed (pipetting up-and-down) and subsequently incubated for 45 minutes at room temperature.

After the incubation, a small sample of each reaction mixture was taken for iTLC (instant thin layer liquid chromatography) to determine radiolabeling reaction yield and the remaining reaction mixtures were transferred to pre-equilibrated PD-10 columns (Vendor) with 250 mM sodium acetate at pH 5.4 for gravity fed desalting. Each PD-10 column took no more than 1.2 mL of reaction mixture (otherwise multiple columns were used). After the contents of the reaction entered the column bed, 1.6 mL of 250 mM sodium acetate at pH 5.4 (formulation buffer) was added; the flow through was discarded. An additional 1.8 mL of formulation buffer was added to the column, and the eluate was collected from each column. Next, approximately 500 uL of each solution was analyzed using a Nanodrop spectrophotometer (ThermoScientific). The final Ab concentration was calculated using the appropriate extinction coefficient and the absorption at 280 nm using the equation:

$$Concentration in mg / mL = Absorption at 280 nm \div Extinction coefficient at 280 nm ( found in Table 6 )$$

The final mass measured in grams was recorded in Table 4. The radioactivity was then measured using the dose calibrator and reported in Table 5. The final material along with the material prior to the PD-10 column treatment, were then analyzed by iTLC. For this assay, 1 uL of each solution was added to the iTLC-SG-Glass microfiber chromatography paper impregnated with silica gel (Agilent Technologies, Cat #SG10001), developed in a TLC chamber with 20 mM citric acid buffer solution. The final material was also analyzed using a SEC-HPLC with UV 280 and radioisotope detector connected in series (Agilent 1260 with Lablogic Radio-TLC/HPLC Detector, SCAN-RAM) using a Superdex 200 column with PBS mobile phase at a flow rate of 0.75 mL/min. The radiotrace was used for the determining radiochemical purity by comparing the integration of the protein peak (˜10 to 16 min) and free ³⁹Zr peak (˜ 25 min). The monomeric purity was determined by comparing the integration of the oligomeric peak (10 min to ˜ 15 min) to the monomer (˜16 min).

The specific activity and protein recovery (%) of each radiolabeled conjugate was determined using the following equations:

$$\begin{matrix}a . Mass of conjugate in mg = concentration in mg / mL \times mass of solution in grams \\ b . Spec if ic activitiy in mCi / mg = activity of vial in mCi \div mass of conjugate in mg \\ c . Pr o tein recovery = starting conjugate mass ( mg ) \div Mass of conjugate in mg\end{matrix}$$

Finally the appearance was noted and recorded in Table 7. Both UV280 and iTLC traces were performed on purified product.

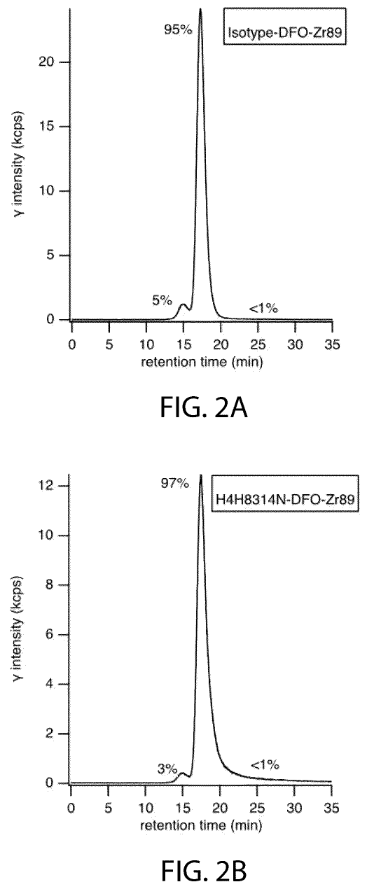

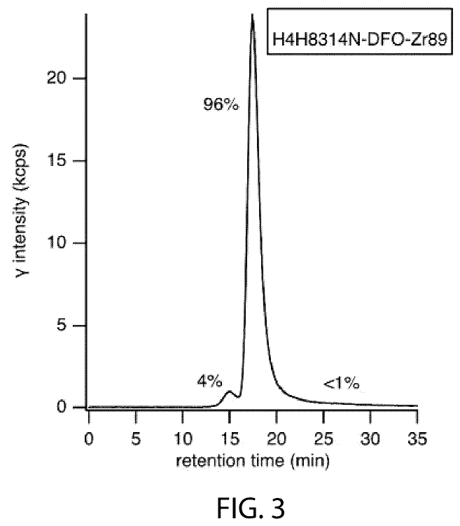

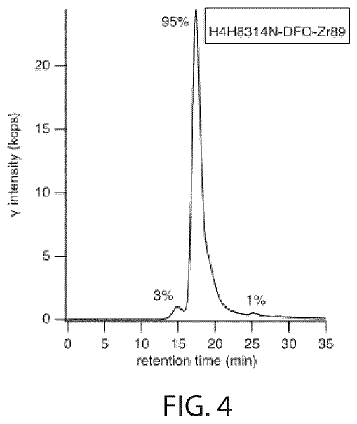

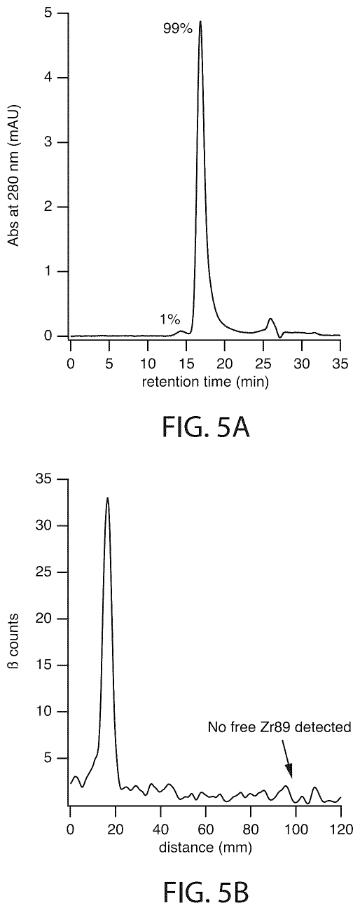

The results are consolidated in Table 7. The radio-SEC-HPLC chromatograms are shown in FIGS. 2-4. An example of UV280 HPLC SEC chromatogram and radio-iTLC is shown in FIG. 5 for the ³⁹Zr radiolabeling, Study 1. The UV280-HPLC SEC chromatogram confirms the highly monomeric product (99%). The radio-iTLC trace was processed with a 7-point binomial smoothing function. The origin and solvent front was approximately 16 and 100 mm, respectively. No detectable ⁸⁹Zr was observed beyond 22 mm and corroborates the radiochemical purity determined by radio-SEC-HPLC SEC in FIG. 2B.

TABLE 4
DFO-antibody conjugate preparation for radiolabeling

| Radio- | | | | | | Total | Final |
|---|---|---|---|---|---|---|---|
| labeling | Study | Radiolabeling | Concentration | | Conjugate | volume | Concentration |
| # | # | Lots | (mg/mL) | DAR * | mass (mg) | (uL) | (mg/mL) |
| 1 | 1 | Isotype- | 3.7 | 1.6 | 250 | 200 | 1.25 |
| | | DFO-⁸⁹Zr | | | | | |
| 2 | 1 | H4H8314N- | 3.34 | 1.2 | 250 | 200 | 1.25 |
| | | DFO-⁸⁹Zr | | | | | |
| 3 | 2 | H4H8314N- | 3.34 | 1.2 | 750 | 600 | 1.25 |
| | | DFO-⁸⁹Zr | | | | | |
| 4 | 3 | Isotype- | 3.7 | 1.6 | 250 | 200 | 1.25 |
| | | DFO-⁸⁹Zr | | | | | |
| 5 | 3 | H4H8314N- | 3.34 | 1.2 | 250 | 200 | 1.25 |
| | | DFO-⁸⁹Zr | | | | | |

* DAR is defined as the DFO to Antibody Ratio

TABLE 5
⁸⁹Zr reaction solution preparation for radiolabeling

| | | | | Add'l | | | | | |
|---|---|---|---|---|---|---|---|---|---|
| | | | | 1 M | 1 M | | | | |
| | | | | oxalic | borate, | 1 M | | | |
| | | | ⁸⁹Zr- | acid | pH 9.0 | HEPES, | Final | Final | Specific |
| Radio- | Study | Radiolabeling | oxalate | added | added | pH 7.2 | Vol | Activity | Activity |
| labeling | # | Lots | (uL) | (uL) | (uL) | (uL) | (uL) | (uCi) | (uCi/uL) |
| 1 | 1 | Isotype- | 50 | 50 | 400 | 500 | 1000 | 1009 | 1.01 |
| | | DFO- | | | | | | | |
| | | ⁸⁹Zr | | | | | | | |
| 2 | 1 | H4H831 | 50 | 50 | 400 | 500 | 1000 | 1000 | 1 |
| | | 4N-DFO-⁸⁹Zr | | | | | | | |
| 3 | 2 | H4H831 | 150 | 150 | 1200 | 1500 | 3000 | 3070 | 1.02 |
| | | 4N-DFO-⁸⁹Zr | | | | | | | |
| 4 | 3 | Isotype- | ~1 | 0 | 0 | 1000 | 1000 | 1680 | 1.68 |
| | | DFO-⁸⁹Zr | | | | | | | |
| 5 | 3 | H4H831 | ~1 | 0 | 0 | 1000 | 1000 | 1640 | 1.64 |
| | | 4N-DFO-⁸⁹Zr | | | | | | | |

TABLE 6
Extinction coefficients for conjugate lots

| Radiolabeling Lot | _{ε280} (AU ml mg⁻¹ cm⁻¹ ) |
|---|---|
| Isotype-DFO-⁸⁹Zr | 1.71 |
| H4H8314N-DFO-⁸⁹Zr | 1.61 |

TABLE 7
Summary of ⁸⁹Zr labeled DFO-Ab conjugates for in vivo imaging
and biodistribution studies

| | | | | Radio- | Mono- | | | |
|---|---|---|---|---|---|---|---|---|
| | | | | chemical | meric | Protein | | Specific |
| Radio- | Study | Conjugate | Appea | Purity* | Purity* | Recovery | Conc. | Activity |
| labeling | # | Lots | r-ance | (%) | (%) | (%) | (mg/mL) | (mCi/mg) |
| 1 | 1 | Isotype- | Clear | >99% | >95% | 60% | 0.106 | 3.35 |
| | | DFO-⁸⁹Zr | | | | | | |
| 2 | 1 | H4H8341 | Clear | >99% | >95% | 63% | 0.121 | 2.75 |
| | | N-DFO- | | | | | | |
| | | ⁸⁹Zr | | | | | | |
| 3 | 2 | H4H8341 | Clear | >99% | >95% | 62% | 0.134 | 3.58 |
| | | N-DFO- | | | | | | |
| | | ⁸⁹Zr | | | | | | |
| 4 | 3 | Isotype- | Clear | >99% | >95% | 66% | 0.074 | 5.38 |
| | | DFO-⁸⁹Zr | | | | | | |
| 5 | 3 | H4H8341 | Clear | >99% | >95% | 74% | 0.084 | 5.13 |
| | | N-DFO- | | | | | | |
| | | ⁸⁹Zr | | | | | | |

*by radio-SEC-HPLC

### Example 4: Immunoreactivity

The immunoreactivity (IR) of the radiolabeled anti-PD-L11 antibody and isotype control antibody was measured as follows. For the initial studies, MC38-cOVA/eGFP-mPD-L1^{-/-}hD-L1^{Tg} cells were used and subsequently LOX-IMVI cells (see detailed description of cell lines in Example 5) were also used in the later study. In these assays, 20 ng of the respective ⁸⁹Zr labeled antibodies were added to 15×10⁶ MC38-cOVA/eGFP-mPD-L1^{-/-}hPD-1^{Tg} or 30×10⁶ LOX-IMVI cells in a final volume of 1 mL. Samples were incubated for 45 minutes with continuous mixing before undergoing 3 washes with media to remove any unbound antibody. The radioactivity of the test cell pellets was then counted in an automatic gamma counter (Wizard 2470, Perkin Elmer) against 2 reference standards containing the same 20 ng of ⁸⁹Zr labeled antibody. The percentage immunoreactivity was determined for the samples using the average of the standards as a measure of total activity.

As seen in Table 8, ⁸⁹Zr labeled anti-PD-L11 antibody retained immunoreactivity following conjugation and radiolabeling, with % IR ranging from 88 to 98% across the studies. The specificity of binding is apparent in the control antibodies having a background % IR of less than 1%.

TABLE 8
Immunoreactivity of ⁸⁹Zr chelated DFO-conjugates

| Study | Study 1 | | Study 2 | | Study 3 | | | |
|---|---|---|---|---|---|---|---|---|
| Cell Line | MC38-cOVA/eGFP- | | MC38-cOVA/eGFP- | | MC38-cOVA/eGFP- | | LOX-IMVI | |
| | mPD-L1^{-/-}hPD-L1^{Tg} | | mPD-L1^{-/-}hPD-L1^{Tg} | | mPD-L1^{-/-}hPD-L1^{Tg} | | | |
| Antibody | ⁸⁹Zr- | ⁸⁹Zr- | ⁸⁹Zr- | ⁸⁹Zr- | ⁸⁹Zr- | ⁸⁹Zr- | ⁸⁹Zr- | ⁸⁹Zr- |
| | Anti-PD- | Control | Anti-PD- | Control | Anti-PD- | Control | Anti-PD- | Control |
| | L1 | | L1 | | L1 | | L1 | |
| Cell | 4048.4 | 29.6 | 8311.9 | na | 6262.4 | 68 | 5587.54 | 65.4 |
| pellet | | | | | | | | |
| activity | | | | | | | | |
| Average | 4536.5 | 6432.4 | 8567.2 | na | 6386.6 | 9544.8 | 6386.6 | 9544.8 |
| Standard | | | | | | | | |
| activity | | | | | | | | |
| Percent | 89.2 | 0.5 | 97.0 | na | 98.1 | 0.7 | 87.5 | 0.7 |
| IR | | | | | | | | |

### Example 5: In Vitro and Ex Vivo Characterization of Human PD-L1 Expression on Tumor Cell Lines

Several tumor cell lines were studied to evaluate the expression level of human PD-L1, aiming at the detection of human PD-L1 expressed endogenously by tumors in vivo in either male NCr nude (Taconic, Hudson NY) mice or in mice that were engineered to be homozygous for the expression of the extracellular domain of human PD-L1 in place of extracellular domain of mouse PD-L1 (PD-L1 Humln mice) on a 75% C57/B16/25% 129 strain background using VelociGene® technology (Valenzuela et al 2003, Nat. Biotechnol. 21: 652-659; US Patent Application Publication US2016/0157469).

Cell lines used in these studies include: 1) a murine colon carcinoma cell line MC38 (obtained from NCI at Frederick, MD, Laboratory of Tumor Immunology and Biology), which has been engineered in house to knock out murine PD-L1, but over-express full-length human PD-L1 and full-length chicken ovalbumin fused with eGFP, thus referred here as MC38-cOVA/eGFP-mPD-L1^{-/-}hPD-L1^{Tg}; 2) several human tumor cell lines: human melanoma cell line LOX-IMVI (endogenous PD-L1 positive line, obtained from NCI at Frederick, MD, Division of Cancer Treatment and Diagnosis, Tumor Repository), human breast cancer cell lines MDA-MB-231 (endogenous PD-L1 positive line) and SK-Br-3 (PD-L1 negative cell line) (both obtained from ATCC). In some cases, human PD-L1 was directly evaluated without any induction in vitro; in some cases, human PD-L1 expression was evaluated with overnight murine or human IFNγ (100 ng/ml) treatment (obtained from Peprotech); in some cases, human PD-L1 was evaluated ex vivo on enzymatically dissociated tumor cells extracted from tumor bearing nude mice or humanized mice. All surface staining of human PD-L1 was performed following a standard protocol. Briefly, tumor cells were washed with PBS once, washed with ice cold staining buffer once, stained with commercial available fluorochrome directly conjugated anti-human PD-L1 antibody (eBioscience, clone MIH1) in staining buffer for 30 minutes on ice in the dark, and then washed with 2 mL of PBS once again. Fixable dye eFluor506 was also included following manufacturer's protocol (eBioscience, Cat #17-5983). Samples were acquired on BD FACSCanto II™ IVD10 equipped with DIVA v8. Data were further analyzed with FlowJo v10.0.6 or above.

PD-L1 expression by MC38-cOVA/eGFP-mPD-L1^{-/-}hPD-L1^{Tg} cells prior to implantation and seven days post implantation in nude mice is shown in Table 9.

TABLE 9
Percentage of human PD-L1 positive
MC38-cOVA/eGFP-mPD-L1^{-/-}hPD-L1^{Tg} cells
prior to implantation and 7 days post implantation in nude mice

| | Isotype staining | hPD-L1 staining |
|---|---|---|
| Prior to implantation | 0.6% | 94.7% |
| Post implantation | 1.09% | 74.0% |

Prior to implantation, a vast majority of MC38-cOVA/eGFP-mPD-L1^{-/-}hPD-L1 T cells were human PD-L1 positive, compared to isotype control staining. Seven days post implantation in nude mice and upon enzymatic and mechanical processing for tumor dissociation, ˜70% of MC38-cOVA/eGFP-mPD-L1^{-/-}hPD-L1^{Tg} cells were still human PD-L1 positive.

PD-L1 expression by MC38-cOVA/eGFP-mPD-L1^{-/-}hPD-L1^{Tg} cells prior to implantation and fourteen days post implantation in PD-L1 humanized mice is shown in Table 10.

TABLE 10
Percentage of human PD-L1 positive
MC38-cOVA/eGFP-mPD-L1^{-/-}hPD-L1^{Tg} cells prior to
implantation and 14 days post implantation in PD-L1 humanized mice

| | Isotype staining | hPD-L1 staining |
|---|---|---|
| Prior to implantation | 0.2% | 92.5% |
| Post implantation | 3.6 | 46.2% |

Prior to implantation, a vast majority of MC38-cOVA/eGFP-mPD-L1^{-/-}hPD-L1 T cells were human PD-L1 positive, compared to isotype control staining. Fourteen days post implantation in PD-1/PD-L1 double humanized mice and upon enzymatic and mechanical processing for tumor dissociation; ˜50% of MC38-cOVA/eGFP-mPD-L1^{-/-}hPD-L1^{Tg} cells were still human PD-L1 positive.

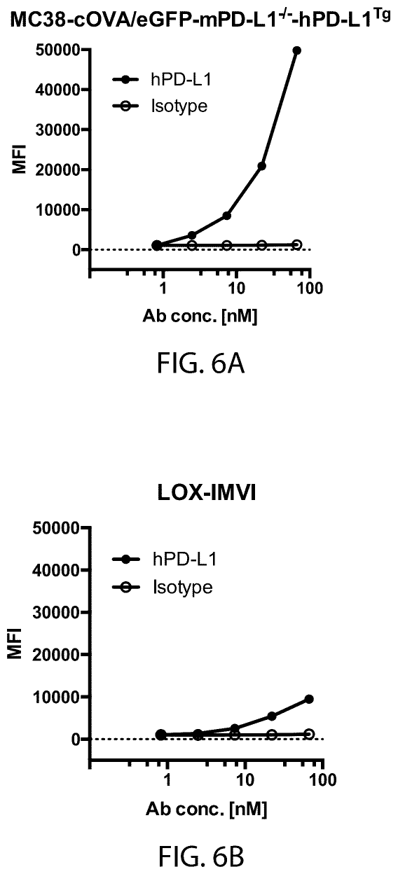

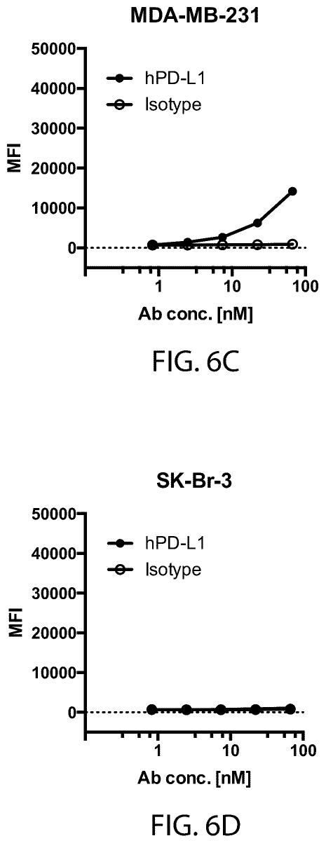

PD-L1 expression by multiple tumor cell lines in vitro is shown in FIG. 6. To evaluate how comparable the expression level of PD-L1 by the engineered cell line (MC38-cOVA/eGFP-mPD-L1^{-/-}hPD-L1^{Tg}) and other human tumor cell lines (LOX-IMVI melanoma cells, MDA-MB-231 breast cancer cells, and SK-Br-3 breast cancer cells) was, dose titration of anti-PD-L1 antibody staining was performed. FIG. 6 illustrates that MC38-cOVA/eGFP-mPD-L1^{-/-}hPD-L1^{Tg} had the highest level of human PD-L1 expression (FIG. 6A) and SK-Br-3 had the lowest expression with no PD-L1 detectable (FIG. 6D), whereas PD-L1 expression by LOX-IMVI and MDA-MB-231 was moderate (about 5 times lower than MC38-cOVA/eGFP-mPD-L1^{-/-}hPD-L1^{Tg}) (FIGS. 6B and 6C).

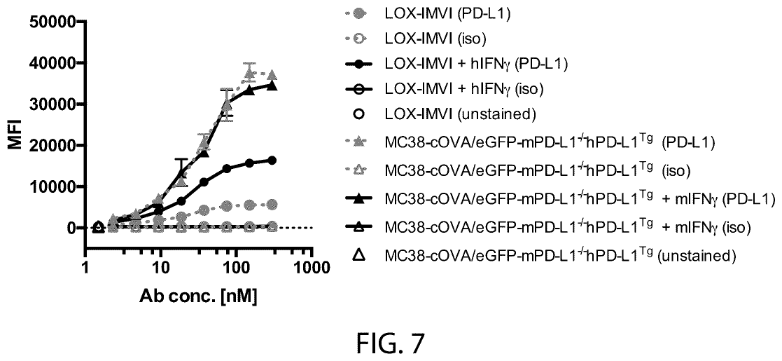

In a second experiment, further comparison between LOX-IMVI and MC38-cOVA/eGFP-mPD-L1^{-/-}hPD-L1^{Tg} was done with or without in vitro treatment by 100 ng/mL of hIFNγ/mIFNγ overnight, respectively. FIG. 7 illustrated that median fluorescence intensity of PD-L1 reached the plateau at ˜150 nM of anti-PD-L1 antibody used for staining. At the baseline, PD-L1 expression by LOX-IMVI was moderate (about 6-7 times lower than MC38-cOVA/eGFP-mPD-L1^{-/-}hPD-L1^{Tg}). Upon treatment with mIFNγ, there was no change for PD-L1 staining on MC38-cOVA/eGFP-mPD-L1^{-/-}hPD-L1^{Tg}, whereas 3-fold increase of human PD-L1 staining was seen in LOX-IMVI after treatment with hIFNγ.

Ex vivo PD-L1 expression by LOX-IMVI and MC38-cOVA/eGFP-mPD-L1^{-/-}hPD-L1^{Tg} cells about three weeks post implantation in nude mice were shown in Tables 11 and 12.

TABLE 11
Percentage of PD-L1 positive
LOX-IMVI and MC38-cOVA/eGFP-mPD-L1^{-/-}hPD-L1^{Tg}
cells ~3 weeks post implantation in nude mice

| | Isotype | hPD-L1 |
|---|---|---|
| | staining | staining |
| LOX-IMVI | 0.2% | 56.6% |
| MC38-cOVA/eGFP-mPD-L1^{-/-}hPD-L1^{Tg} | 0.2% | 96.2% |

TABLE 12
Mean fluorescence intensity of PD-L1 by
LOX-IMVI and MC38-cOVA/eGFP-mPD-L1^{-/-}hPD-L1^{Tg} cells
~3 weeks post implantation in nude mice

| | Tumor 1 | Tumor 2 |
|---|---|---|
| LOX-IMVI | 8479.1 | 12121.5 |
| MC38-cOVA/eGFP-mPD-L1^{-/-}hPD-L1^{Tg} | 49589.1 | 51445.0 |

Upon enzymatic and mechanical processing to allow for tumor dissociation, cells were stained with the anti-PD-L1 antibody (20 μg/mL). The PD-L1 expression level on LOX-IMVI was about 5 times lower than that on MC38-cOVA/eGFP-mPD-L1^{-/-}hPD-L1^{Tg} tumor cells.

### Example 6: Selective Localization of Radiolabeled Anti-PD-L1 Antibody to hPD-L1 Positive Tumors in Nude Mice

To determine the in vivo localization of anti-PD-L1 antibody, Zirconium-89 labeled DFO-antibody conjugate was administered intravenously to nude mice bearing PD-L1 positive tumors.

The tumor line used for the study was a murine colon carcinoma cell-line referred to as MC38-cOVA/eGFP-mPD-L1^{-/-}hPD-L1^{Tg}, which has been engineered to knock out murine PD-L1 off the wild type MC38, but over-express full-length human PD-L1 and full-length chicken ovalbumin fused with eGFP. For the second study of tumors with endogenous expression of human PD-L1, the human melanoma cell line LOX-IMVI was used to establish tumors in vivo for subsequent anti-PD-L1 antibody localization studies.

The exemplary radiolabeled anti-PD-L1 antibody used for this study was H1H8314N, comprising HCVR/LCVR of SEQ ID NOs: 82/90.

For the first study, 1×10⁶ MC38-cOVA/eGFP-mPD-L1−/−hPD-L1^{Tg} cells were implanted subcutaneously into the left flank of male 8-10 week old NCr nude mice (Taconic, Hudson NY). For LOX-IMVI tumors, 1×10⁶ cells were implanted subcutaneously into the left flank of male 8-10 week old NCr nude mice. Once tumors had reached an average volume of 50-150 mm³ (˜Day 7-10), mice were randomized into groups, and dosed with either ⁸⁹Zr labeled anti-PD-L1 DFO-antibody conjugate (H1H8314N) or a ⁸⁹Zr labeled non-binding isotype control DFO-antibody conjugate. The nude mice bearing MC38-cOVA/eGFP-mPD-L1−/−hPD-L1^{Tg} tumors received 50±1 uCi of ⁸⁹Zr labeled antibody with a protein dose ˜0.6 mg/kg. In the study using mice bearing LOX-IMVI tumors, mice received 35±1 uCi of ⁸⁹Zr labeled antibody with a final antibody dose of 0.3 or 1 mg/kg.

PET imaging of antibody localization was assessed 6 days after administration of the antibodies. A Sofie Biosciences G8 PET/CT (Sofie Biosciences and Perkin Elmer) was used to acquire images). The instrument was pre-calibrated for detection of ⁸⁹Zr prior to image acquisition. The energy window ranged from 150 to 650 keV with a reconstructed resolution of 1.4 mm at the center of the field of view. Mice underwent induction anesthesia using isoflurane and were kept under continuous flow of isoflurane during imaging. Static 10-minute images were acquired using the G8 acquisition software and subsequently reconstructed using the pre-configured settings. Image data was corrected for decay and other parameters. CT images were acquired following PET acquisition and later co-registered with the PET images. Images were prepared using VivoQuant post-processing software (inviCRO Imaging Services).

For bio distribution, mice were euthanized at the final time-point (5-6 days post-dosing) and blood was collected via cardiac puncture. Tumors and normal tissues were then excised and placed in counting tubes. Weight for each sample were measured and recorded. Count data for ⁸⁹Zr in CPM was then collected by measuring samples on an automatic gamma counter (Wizard 2470, Perkin Elmer). The percent-injected dose per gram (% ID/g) was calculated for each sample using standards prepared from the injected material.

The average % ID/g for each antibody is presented in Table 13.

TABLE 13
Average % ID/g in analyzed tissues

| | | | ⁸⁹Zr-Isotype Control | |
|---|---|---|---|---|
| | ⁸⁹Zr-H1H8314N | | Antibody | |
| | AVERAGE | STDEV | AVERAGE | STDEV |
| SAMPLE | % ID/g | % ID/g | % ID/g | % ID/g |
| LIVER | 3.1 | 0.4 | 0.9 | 0.9 |
| SPLEEN | 4.4 | 1.1 | 1.5 | 1.3 |
| KIDNEY | 4.0 | 0.7 | 1.4 | 1.6 |
| BONE | 5.1 | 2.6 | 1.7 | 1.6 |
| LUNG | 5.1 | 1.1 | 2.5 | 3.0 |
| HEART | 2.4 | 0.2 | 1.3 | 1.4 |
| BLOOD | 7.6 | 1.6 | 3.8 | 4.6 |
| THYMUS | 5.3 | 3.0 | 2.8 | 2.2 |
| MC38-cOVA/eGFP- | 55.3 | 12.2 | 3.0 | 3.3 |
| mPD-L1^{-/-}hPD-L1^{Tg} | | | | |
| S. BOWEL | 1.5 | 0.3 | 0.6 | 0.6 |

From this, the clear high uptake in MC38-cOVA/eGFP-mPD-L1−/−hPD-L1^{Tg} tumors was apparent over other normal tissues, with tumor uptake of 55.3% ID/g being significantly higher than the next highest uptake of 5.3% ID/g observed in the thymus. Tumor uptake was 7.3-fold and 17.8-fold higher than activity in blood and liver, respectively. The specificity of anti-PD-L1 uptake into tumor (55.3% ID/g) was apparent as compared to significantly reduced tumor uptake of 3% observed for the non-binding isotype control antibody. Pilot PET imaging performed here demonstrated a clear localization of the ⁸⁹Zr labeled anti-PD-L1 DFO-antibody conjugate to the MC38-cOVA/eGFP-mPD-L1−/−hPD-L1^{Tg} tumors. Little background signal was observed in the animals at this Day 6 post-dosing time-point. In contrast to the clear tumor localization that was apparent using anti-PD-L1 antibody, only faint background activity was apparent in imaging of the control antibody in this model. Imaging clearly indicated high, specific uptake of anti-PD-L1 antibody in human PD-L1 positive tumor, showing the localization of ⁸⁹Zr radiolabeled anti-PD-L1 antibody to a MC38-cOVA/eGFP-mPD-L1−/−hPD-L1^{Tg} tumor in an NCr nude mouse.

In a second study, the ability of anti-PD-L1 antibody to selectively target tumors expressing endogenous levels of human PD-L1 antigen was assessed. Here, mice bearing human LOX-IMVI melanoma tumors received ⁸⁹Zr labeled antibody at doses of 0.3 and 1 mg/kg. Again, blood, tumor and tissues were taken at Day 6 post-injection and the % ID/g for the samples was calculated. The average % ID/g for each antibody is presented in Table 14.

TABLE 14
Average % ID/G in analyzed tissues from second study (LOX-IMVI tumors)

| | ⁸⁹Zr-DFO- | | ⁸⁹Zr-DFO- | | ⁸⁹Zr-Isotype | |
|---|---|---|---|---|---|---|
| | H1H8314N | | H1H8314N | | control antibody | |
| | 0.3 mg/kg | | 1 mg/kg | | 1 mg/kg | |
| | AVERAGE | STDEV | AVERAGE | STDEV | AVERAGE | STDEV |
| SAMPLE | % ID/g | % ID/g | % ID/g | % ID/g | % ID/g | % ID/g |
| LIVER | 2.9 | 0.3 | 3.3 | 0.2 | 3.9 | 0.3 |
| SPLEEN | 4.2 | 0.2 | 4.3 | 0.9 | 4.2 | 0.7 |
| KIDNEY | 4.3 | 0.4 | 4.3 | 0.8 | 3.4 | 0.4 |
| BONE | 3.2 | 0.6 | 2.7 | 0.5 | 3.6 | 0.4 |
| LUNG | 5.7 | 1.0 | 6.6 | 1.6 | 5.9 | 1.2 |
| HEART | 3.2 | 0.8 | 3.2 | 0.4 | 2.9 | 0.6 |
| BLOOD | 8.1 | 1.4 | 9.5 | 1.0 | 11.1 | 6.2 |
| THYMUS | 5.3 | 2.3 | 5.6 | 0.7 | 4.9 | 1.4 |
| LOX-IMVI | 20.6 | 2.7 | 10.6 | 2.6 | 12.0 | 1.8 |
| TUMOR | | | | | | |
| S. BOWEL | 1.5 | 0.2 | 1.8 | 0.4 | 2.0 | 0.3 |

At the lower 0.3 mg/kg dose, clear targeting to tumor over normal tissues was observed, with a 20.6% ID/g observed in the LOX-IMVI tumors. When mice received the higher 1 mg/kg dose, reduced tumor uptake 10.6% ID/g of was observed relative to the 0.3 mg/kg level. This suggests that the higher protein dose and possibly the subsequent higher fraction of unlabeled antibody led to blocking of tumor uptake by the ³⁹Zr labeled anti-PD-L1 antibody. In accordance with this, PET imaging conducted immediately prior to the biodistribution study also showed that uptake of anti-PD-L1 antibody at the 1 mg/kg dose was roughly equivalent to that of the control antibody. At the lower dose of 0.3 mg/kg, a clear increase in tumor localization of the anti-PD-L1 antibody was apparent relative to control antibody. Overall, the PET images and the biodistribution data demonstrate specific targeting of the LOX-IMVI tumors at the 0.3 mg/kg dose of anti-PD-L1 antibody.

### Example 7: Selective Localization of Radiolabeled Anti-PD-L1 Antibody to hPD-L1 Positive Tumors in Mice

This Example describes the in vivo localization of a Zirconium-89 labeled DFO-anti-PD-L1 antibody conjugate in mice humanized for PD-L1. The exemplary antibody used in this Example was H1H8314N, comprising HCVR/LCVR of SEQ ID NOs: 82/90.

Mice humanized for PD-L1 were engineered using VelociGene® technology (Valenzuela et al 2003, Nat. Biotechnol. 21: 652-659; US Patent Application PublicationUS2016/0157469).

The tumor line used was a murine colon carcinoma cell-line referred to as MC38-cOVA/eGFP-mPD-L1^{-/-}hPD-L1^{Tg}, engineered in-house to express full-length chicken ovalbumin fused with eGFP and to knock out murine PD-L1 off the wild type MC38, but over-express full-length human PD-L1.

1×10⁶ cells of MC38-cOVA/eGFP-mPD-L1^{-/-}hPD-L1^{Tg} were implanted subcutaneously into the left flank of male humanized PD-L1 mice. Once tumors had reached an average volume of 50-150 mm³ (˜Day 7), mice were randomized into groups, and dosed with either ⁸⁹Zr labeled anti-PD-L1 DFO-antibody conjugate or a ⁸⁹Zr labeled non-binding isotype control DFO-antibody conjugate. The mice received 50±1 uCi of ⁸⁹Zr labeled antibody with a final protein dose of 1 or 3 mg/kg.

PET imaging of antibody localization was assessed 6 days after administration of the antibodies. A Sofie Biosciences G8 PET/CT (Sofie Biosciences and Perkin Elmer) was used to acquire images). The instrument was pre-calibrated for detection of ⁸⁹Zr prior to image acquisition. The energy window ranged from 150 to 650 keV with a reconstructed resolution of 1.4 mm at the center of the field of view. Mice underwent induction anesthesia using isoflurane and were kept under continuous flow of isoflurane during imaging. Static 10-minute images were acquired using the G8 acquisition software and subsequently reconstructed using the pre-configured settings. Image data was corrected for decay and other parameters. CT images were acquired following PET acquisition and later co-registered with the PET images. Images were prepared using VivoQuant post-processing software (inviCRO Imaging Services).

For biodistribution, mice were euthanized at the final time-point (5-6 days post-dosing) and blood was collected via cardiac puncture. Tumors and normal tissues were then excised and placed in counting tubes. Weight for each sample were measured and recorded. Count data for ⁸⁹Zr in CPM was then collected by measuring samples on an automatic gamma counter (Wizard 2470, Perkin Elmer). The percent-injected dose per gram (% ID/g) was calculated for each sample using standards prepared from the injected material.

### Results

Humanized PD-L1 mice bearing MC38-cOVA/eGFP-mPD-L1^{-/-}hPD-L1^{Tg} tumors received ⁸⁹Zr labeled anti-PD-L1 DFO-antibody conjugate at a final antibody dose of 1 or 3 mg/kg. Blood, tumor and tissues were taken and weighed at Day 6 post-injection and the % ID/g for the samples was calculated based on the counts from each sample. The average % ID/g for dose at 1 and 3 mg/kg is presented in Table 15 and Table 16 respectively.

TABLE 15
Average % ID/g in analyzed tissues of anti-PD-L1 antibody at 1 mg/kg

| | AVERAGE | |
|---|---|---|
| SAMPLE | % ID/g | STDEV % ID/g |
| LIVER | 8.6 | 1.5 |
| SPLEEN | 14.1 | 1.1 |
| KIDNEY | 7.8 | 1.0 |
| BONE | 4.5 | 1.4 |
| LUNG | 7.9 | 3.0 |
| HEART | 4.3 | 1.1 |
| BLOOD | 9.1 | 4.6 |
| THYMUS | 9.7 | 3.5 |
| MC38-cOVA/eGFP- | 34.1 | 18.0 |
| mPD-L1^{-/-}hPD-L1^{Tg} | | |
| S. BOWEL | 2.4 | 0.9 |

At the 1 mg/kg dose level, clear tumor targeting of the MC38-cOVA/eGFP-mPD-L1^{-/-}hPD-L1^{Tg} tumors is apparent with a % ID/g of 34.1% despite the expression of PD-L1 in normal tissues in these humanized mice. At this dose, some localization of the ⁸⁹Zr labeled anti-PD-L1 antibody was apparent in the spleen, where antibody uptake of 14.1% ID/g was observed. Such uptake is expected because of the normal expression of human PD-L1 in place of mouse PD-L1 expression of human PD-L1 in the spleen. At the 3 mg/kg antibody dose, localization of ⁸⁹Zr-DFO-anti-PD-L1 antibody conjugate to the spleen was reduced, as uptake now averaged 9.7% ID/g in mice that received this antibody dose (Table 16).

TABLE 16
Average % ID/g in analyzed tissues of anti-PD-L1 antibody at 3 mg/kg

| | AVERAGE | |
|---|---|---|
| SAMPLE | % ID/g | STDEV % ID/g |
| LIVER | 6.7 | 1.4 |
| SPLEEN | 9.7 | 1.3 |
| KIDNEY | 7.0 | 1.1 |
| BONE | 3.6 | 0.6 |
| LUNG | 11.0 | 1.0 |
| HEART | 4.7 | 0.7 |
| BLOOD | 12.4 | 2.1 |
| THYMUS | 7.6 | 0.5 |
| MC38-cOVA/eGFP-mPD- | 28.7 | 13.1 |
| L1^{-/-}hPD-L1^{Tg} | | |
| S. BOWEL | 0.4 | 0.2 |

Clear tumor targeting was still observed at the 3 mg/kg dose, with an average of 28.7% ID/g taken up by the MC38-cOVA/eGFP-mPD-L1^{-/-}hPD-L1^{Tg} tumors. Therefore although reduced normal tissue localization was apparent in imaging the 3 mg/kg dose, clear localization of anti-PD-L1 labeled antibody to the MC38-cOVA/eGFP-mPD-L1^{-/-}hPD-L1^{Tg} tumors remained clear at this dose. Overall, these results indicate that clear targeting of the MC38-cOVA/eGFP-mPD-L1^{-/-}hPD-L1^{Tg} tumors was possible in mice expressing PD-L1 on regular sites of normal tissue expression.

The results from the studies performed here clearly demonstrate that anti-PD-L1 antibody labeled with ⁸⁹Zr can significantly and specifically localize to tumors. One may envisage a scenario where the anti-PD-L1 antibody is used in the selection of patients with PD-L1 positive tumors for subsequent treatment with inhibitors of the PD-1/PD-L1 signaling axis.

### Example 8: Scaled-Up Manufacturing Process for Producing DFO-Anti-PD-L1 Antibody Conjugates

This example details the scaled-up manufacturing process for preparing the anti-PD-L1 antibody to be suitable for radiolabeling by attaching p-SCN-bn-Deferoxamine (DFO) to the anti-PD-L1 antibody (mAb, H4H8314N) described herein: (1) ultrafiltration and diafiltration (UFDF) processes prior to mAb conjugation removes excipients that inhibit the conjugation process; (2) following the pre-conjugation UFDF, conjugation of the mAb with p-SCN-Bn-deferoxamine is performed to produce DFO-mAb conjugates; and (3) a post-conjugation UFDF to remove residual salts provides a suitable concentration, excipient level, and pH of the conjugated monoclonal antibody. The resulting DFO-mAb conjugates are then provided in a buffered state with improved stability for subsequent formulation.

### (1) Pre-Conjugation Ultrafiltration and Diafiltration (UFDF)

100 g anti-PD-L1 antibody was buffer exchanged into a 5 mM acetate buffer solution having a pH of 5.50 using a Sius Prostream (TangenX Technology Corporation) membrane (membrane capacity of ≤500 g/m²) to remove residual salts prior to conjugation. The process volume was reduced to further concentrate the antibody, then the antibody was sterile filtered using a Sartopore 2 (Sartorius) membrane having a 0.45/0.2 μm (heterogeneous PES double layer) or equivalent pore size. The acetate buffer temperature was kept at a target temperature of 20±5° C. The solutions were well mixed.

### (2) Conjugation

The concentrated and filtered antibody (20 g) was transferred into a conjugation vessel containing an amine free carbonate buffer system (56 mM Carbonate, 167 mM Sodium Chloride, pH 9.40) resulting in negligible levels of residual acetate. DFO (25 mM p-SCN-Bn-Deferoxamine) was solubilized in DMSO and added to the conjugation vessel, along with additional DMSO such that the DMSO was present in a final amount of 5%. DFO was added in molar excess at a ratio of 4.5:1 DFO to mAb. The total reaction volume equaled 2.0 L. The buffer system was mixed throughout the addition of the reaction ingredients and throughout the reaction time.

The reaction temperature was controlled for specific time by using an equation which relates temperature to reaction time. In this instance, the reaction temperature was held at 18° C. for 120 minutes. The reaction was quenched by the addition of 2M acetic acid (23 mL/L), resulting in the solution having a pH of 6.

### (3) Post-Conjugation UFDF

After the conjugation step, the quenched DFO-mAb conjugation solution was buffer exchanged into histidine buffer (10 mM Histidine, pH 5.50 with 0.0005% (w/v) super refined polysorbate 80 added as a shear protectant) to remove residual process salts, DMSO, and unreacted DFO. Once diafiltered, the solution was then concentrated and subsequently formulated. The histidine buffer was selected for long term storage of protein at −80° C. The same Sius Prostream membrane mentioned in step (1) was used in the final UFDF step. The resulting concentrated DFO-mAb conjugate solution was sterile filtered using the Sartopore 2 filter mentioned above.

UV-DAR (target of 1.5) and protein concentration determination was performed as described in Example 2.

TABLE 17
Molar Extinction Coefficients and Molecular Weight

| | MW | ε280 | ε252 |
|---|---|---|---|
| Antibody | (g mol⁻¹) | (L g⁻¹cm⁻¹) | (L g⁻¹cm⁻¹) |
| H4H8314N | 144984 | 211480 | 80172 |

### Example 9: Predicted Whole Body and Tissue Exposure of Radioactivity in Human Subjects to be Given an IV Dose of ⁸⁹Zr-DFO-Anti-PD-L1 Antibody Conjugate

The purpose of the following experiment was to estimate the predicted whole body and tissue exposures to radioactivity in human subjects due to an intravenous (IV) dose of ⁸⁹Zr-DFO-anti-PD-L1 antibody conjugate. The exemplary anti-PD-L1 antibody used in the radiolabeled conjugate was H4H8314N.

### Characterization of Radioimmunoconjugates

Anti-PD-L1 immunoconjugate (DFO-Ab) and isotype control immunoconjugate (DFO-IgG4^{P} Control) were radiolabeled and purified for use in in vivo imaging and biodistribution studies. SEC-HPLC analysis and a MC38/mPD-L1^{-/-}/hPD-L1 (murine MC38 colon adenocarcinoma cells engineered to knock out mouse PD-L1 and stably express human PD-L1) cell-based in vitro assay were performed to characterize the resultant radioimmunoconjugates.

### Monomeric and Radiochemical Purity

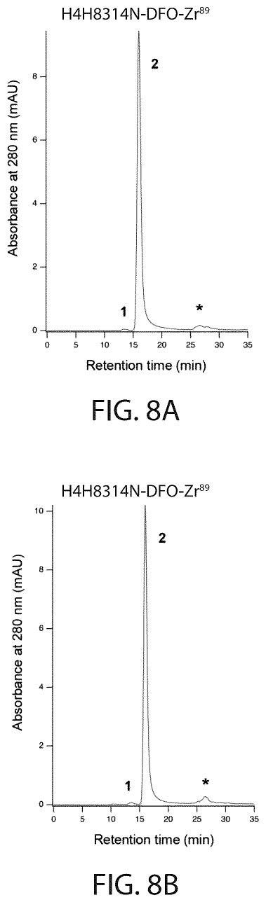

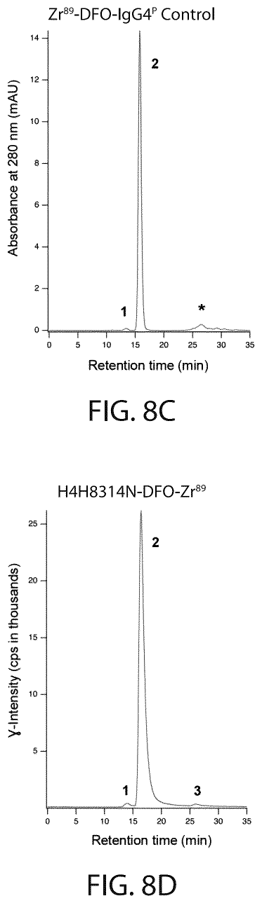

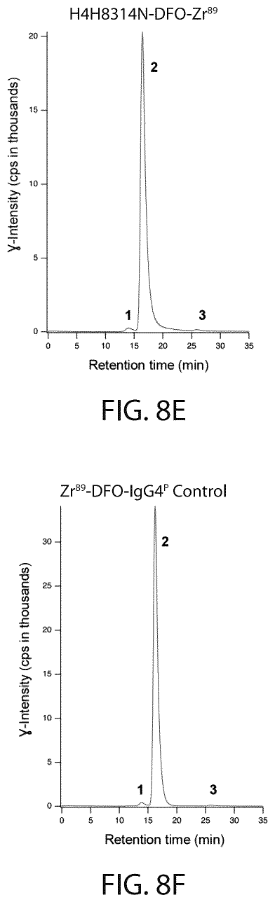

SEC-HPLC using UV- and γ-emission detectors was performed to assess monomeric and radiochemical purity. Results for radioimmunoconjugate preparations of ⁸⁹Zr-DFO-anti-PD-L1 antibody conjugate and of isotype control radioimmunoconjugate ⁸⁹Zr-DFO-IgG4^{P} are shown in FIG. 8.

Analysis of chromatograms for absorption at 280 nm was performed to evaluate the relative amounts of high molecular weight (HMW) and monomeric protein in the radioimmunoconjugate preparations. As summarized in Table 18, the monomeric peaks (a readout of monomeric purity) constitute 99.6, 99.2, and 98.6%, respectively, of the total protein peak area for preparations of ⁸⁹Zr-DFO-and-PD-L1 antibody conjugate and isotype control ⁸⁹Zr-DFO-gG4^{P}; low levels of HMW species (0.4, 0.8, and 1.4%, respectively) were also detected. Low molecular weight (LMW) species were not observed for any of the tested samples.

Analysis of radio-chromatograms for γ-emission was performed to evaluate the relative amounts of ⁸⁹Zr incorporated into radioimmunoconjugates compared with unincorporated ⁸⁹Zr (such as free ⁸⁹Zr or ⁸⁹Zr chelated with free DFO-derivatives). As summarized in Table 18, the peaks for unincorporated ⁸⁹Zr constitute 216.1% of the total γ-emission peak area, while the combined peaks for radiolabeled monomeric and HMW species (a readout of radiochemical purity) constitute 98.9, 99.5, and 99.5%, respectively, of the total γ-emission peak area for preparations of ⁸⁹Zr-DFO-anti-PD-L1 antibody conjugate and isotype control ⁸⁹Zr-DFO-IgG4^{P}.

TABLE 18
Summary of SEC-HPLC Data

| | | Approximate | Peak Area (%) | |
|---|---|---|---|---|
| | | Retention | UV- | Radio- |
| Peak | | Time | Chroma- | Chroma- |
| Number | Species | (min) | togram | togram |

⁸⁹Zr-DFO-H4H8314N Study 1

| | | | | |
|---|---|---|---|---|
| 1 | HMW | 13 | 0.4 | 1.1 |
| 2 | Monomer | 16 | 99.6 | 97.8 |
| 3 | Unincorporated ⁸⁹Zr | 26 | n/a | 1.1 |

⁸⁹Zr-DFO-H4H8314N Study 2

| | | | | |
|---|---|---|---|---|
| 1 | HMW | 14 | 0.8 | 1.3 |
| 2 | Monomer | 16 | 99.2 | 98.2 |
| 3 | Unincorporated ⁸⁹Zr | 26 | n/a | 0.5 |

⁸⁹Zr-DFO-IgG4^{P} Control

| | | | | |
|---|---|---|---|---|
| 1 | HMW | 13 | 1.4 | 1.5 |
| 2 | Monomer | 16 | 98.6 | 98.0 |
| 3 | Unincorporated ⁸⁹Zr | 26 | n/a | 0.5 |

Numerical values for SEC-HPLC analysis graphically represented in FIG. 8. UV-chromatogram indicated the chromatogram for absorption at 280 nm and radio-chromatogram indicates the chromatogram for intensity of γ-emission. HMW: high molecular weight; n/a: not applicable.

### Immunoreactivity

The immunoreactivity, a measure of the percent of radiolabeled, conjugated antibody that is capable of binding its antigen, was determined by incubating ⁸⁹Zr-DFO-anti-PD-L1 antibody conjugate with MC38/mPD-L1^{-/-}/hPD-L1 cells. The 2 tested lots of ⁸⁹Zr-DFO-anti-PD-L1 antibody conjugate demonstrated 84.5 and 88.8% immunoreactivity on MC38/mPD-L1^{-/-}/hPD-L1 cells (Table 19). Background, nonspecific immunoreactivity of 8.8% was observed for the isotype control radioimmunoconjugate.

TABLE 19
Immunoreactivity of ⁸⁹Zr labeled anti-PD-L1 DFO-antibody
conjugate and isotype control ⁸⁹Zr-DFO-IgG4^{P}

| Radioimmunoconjugate | Immunoreactivity |
|---|---|
| ⁸⁹Zr-DFO-anti-PD-L1 antibody | 84.5% |
| conjugate (lot 1) | |
| ⁸⁹Zr-DFO-anti-PD-L1 antibody | 88.8% |
| conjugate (lot 2) | |
| isotype control ⁸⁹Zr-DFO-IgG4^{P} | 8.8% |

In conclusion, two separate lots of ⁸⁹Zr-DFO-anti-PD-L1 antibody conjugate showed high immunoreactivity, percentage of monomer, and radiochemical purity.

### ⁸⁹Zr-DFO-anti-PD-L1 Biodistribution in Mice

This experiment evaluated the biodistribution of the anti-human PD-L1 radioimmunoconjugate, ⁸⁹Zr-DFO-anti-PD-L1 antibody conjugate, over time following administration of a single 50 μCi (1 mg/kg) intravenous (IV) dose to PD-L1/PD-1-humanized mice (PD-1hu/huPD-L1hu/hu). Since H4H8314N does not bind mouse PD-L1, the portion of the mouse PD-L1 gene encoding the PD-L1 ectodomain was replaced by the corresponding human sequence in PD-1hu/hu-PD-L1hu/hu mice. In this strain, the ectodomain of mouse PD-1 was similarly humanized. These mice were not subjected to immune/inflammatory challenge, and are therefore expected to have unstimulated levels of PD-L1 expression on immune cells. Two groups of 8 animals each were sacrificed 6 days (144 hours) or 10 days (240 hours) post dosing, blood was collected and the following tissues were harvested: heart, lungs, liver, spleen, kidneys, stomach, small intestine, caecum, large intestine, bone (femur), thymus, muscle, bladder, and brain. The percentage of radioactivity of the total injected dose (% ID) localized to specific tissues or blood was determined and reported as average % ID per gram (% ID/g) of tissue. In advance of sacrifice, immuno-PET/computed tomography (CT) images were acquired 1, 24, 48, 72, 144, 192 (10-day group only), and 240 (10-day group only) hours post dosing from the same animals.

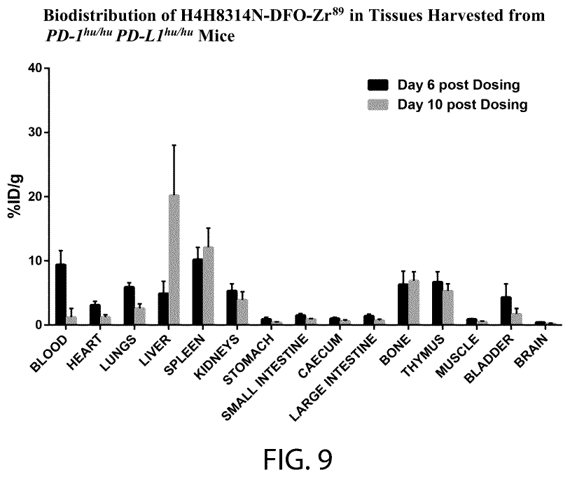

Relative to ⁸⁹Zr levels in blood, uptake of ⁸⁹Zr-DFO-anti-PD-L1 antibody conjugate into specific tissues was negligible throughout the 10-day study period, as evaluated by ex vivo tissue analysis (Table 20 and FIG. 9) and in vivo imaging. Compared with blood (9.4±2.2% ID/g), all harvested tissues, with the exception of spleen, demonstrated lower ⁸⁹Zr levels (≤6.7% ID/g) on day 6 post dosing. A small degree of target-mediated ⁸⁹Zr-DFO-anti-PD-L1 antibody conjugate uptake (10.2±1.9% ID/g) was observed in the spleen, in agreement with PD-L1 expression on splenocytes, as demonstrated by flow cytometry. At 10 days post-dosing, ⁸⁹Zr levels in blood had decreased 7.8-fold relative to day 6 post dosing, suggesting a mouse-anti-human antibody (MAHA) response affecting ⁸⁹Zr-DFO-anti-PD-L1 antibody conjugate levels. This observed MAHA response is likely due to the fact that the target, PD-L1, is expressed on antigen-presenting cells (Francisco, 2010), leading to the presentation of the human antibody to the mouse immune system and subsequent MAHA formation. In parallel, ⁸⁹Zr levels in the liver were 4.1-fold increased on day 10 compared with day 6 post dosing, possibly as a result of MAHA/⁸⁹Zr-DFO-anti-PD-L1 antibody conjugate immune complex (IC) formation and subsequent liver-mediated IC clearance (Rojko, 2014). Whole animal in vivo PET imaging did not uncover marked tissue-specific uptake of ⁸⁹Zr-DFO-anti-PD-L1 antibody conjugate beyond a low signal for spleen and the MAHA-mediated accumulation in the liver described above.

In summary, marked target-mediated uptake of ⁸⁹Zr-DFO-anti-PD-L1 antibody conjugate into specific tissues above ⁸⁹Zr levels in blood was not observed over a 6-day period in PD-L1/PD-1-humanized mice administered a single IV dose of 1 mg/kg (50 μCi) of ⁸⁹Zr-DFO-anti-PD-L1 antibody conjugate with the exception of the spleen, where a small degree of target-mediated uptake was observed in agreement with the demonstrated expression of PD-L1 on splenocytes. Data collected beyond day 6 until the end of the study on day 10 post dosing were affected by a MAHA response.

TABLE 20
Average Ex Vivo Biodistribution Data

| | ⁸⁹Zr Levels on Day 6 | | ⁸⁹Zr Levels on Day 10 | |
|---|---|---|---|---|
| | post Dosing (% ID/g) | | post Dosing (% ID/g) | |
| Tissue | Average | SD | Average | SD |
| Blood | 9.4 | 2.2 | 1.2 | 1.4 |
| Heart | 3.1 | 0.6 | 1.2 | 0.4 |
| Lungs | 5.9 | 0.7 | 2.6 | 0.7 |
| Liver | 4.9 | 1.9 | 20.2 | 7.8 |
| Spleen | 10.2 | 1.9 | 12.1 | 3.0 |
| Kidneys | 5.3 | 1.1 | 3.9 | 1.3 |
| Stomach | 0.9 | 0.3 | 0.4 | 0.1 |
| Small Intestine | 1.5 | 0.3 | 0.9 | 0.1 |
| Caecum | 1.0 | 0.2 | 0.6 | 0.2 |
| Large Intestine | 1.4 | 0.3 | 0.7 | 0.2 |
| Bone (Femur) | 6.3 | 2.1 | 6.9 | 1.4 |
| Thymus | 6.7 | 1.6 | 5.3 | 1.1 |
| Muscle | 0.9 | 0.1 | 0.5 | 0.1 |
| Bladder | 4.3 | 2.1 | 1.7 | 0.9 |
| Brain | 0.4 | 0.1 | 0.2 | 0.1 |

Abbreviation: % ID/g = Percent injected dose per gram (of tissue)

### Estimates of Whole Body and Tissue Exposures to Radioactivity in Humans

This experiment used PET/CT image data for four PD-1/PD-L1-humanized male mice and four PD-1/PD-L1-humanized female mice imaged at 1, 24, 48, 72, 144, 192, and 240 hours following single IV administration of 50 μCi (1 mg/kg) of ⁸⁹Zr-DFO-anti-PD-L1 antibody conjugate. The data generated by administration of this clinically relevant dose was used in calculating estimates of human exposure to radioactivity. Tissue concentration data was determined using volume of interest (VOI) analysis.

For radiation dosimetry estimation, the mean residence time was determined for the following regions: brain, stomach contents, heart contents, kidneys, liver, lungs, muscle, red marrow, spleen, bladder contents, and remainder of body. These mean residence time values were used as an input into the OLINDA/EXM 1.1 software program to estimate the mean absorbed tissue doses and effective dose in humans.

The effective human dose for ⁸⁹Zr-DFO-anti-PD-L1 antibody conjugate was estimated to be 0.513 mSv/MBq (millisievert/megabecquerel) in the adult male and 0.622 mSv/MBq in the adult female. The organs predicted to have the highest absorbed dose in humans were the spleen and liver. The estimated absorbed dose in the spleen was 0.856 mSv/MBq in the adult male and 1.12 mSv/MBq in the adult female. The estimated absorbed dose in the liver was 0.764 mSv/MBq in the adult male and 0.974 mSv/MBq in the adult female.

Average decay-corrected percent of the injected dose per mL (DC % ID/mL) values for male and female mice (n=4 male, n=4 female) for each VOI are summarized in Table 21.

TABLE 21
Biodistribution Data

| | Average Decay-corrected Percent Injected Dose Per mL (DC % ID/mL) ± SD | | | | | | | | | |
|---|---|---|---|---|---|---|---|---|---|---|
| Time (h) | 1 | | 24 | | 48 | | 72 | | 144 | |
| Sex | Female | Male | Female | Male | Female | Male | Female | Male | Female | Male |
| Brain | 1.365 ± | 1.190 ± | 0.903 ± | 0.538 ± | 0.640 ± | 0.548 ± | 0.685 ± | 0.623 ± | 0.465 ± | 0.398 ± |
| | 0.115 | 0.050 | 0.115 | 0.071 | 0.079 | 0.218 | 0.096 | 0.224 | 0.231 | 0.073 |
| Lungs | 12.503 ± | 12.498 ± | 8.293 ± | 7.155 ± | 6.715 ± | 5.888 ± | 6.060 ± | 5.558 ± | 4.863 ± | 4.585 ± |
| | 1.146 | 0.414 | 0.635 | 1.175 | 0.370 | 0.990 | 0.708 | 0.385 | 0.316 | 0.339 |
| Liver | 12.298 ± | 12.078 ± | 9.058 ± | 7.200 ± | 8.113 ± | 6.125 ± | 7.838 ± | 6.203 ± | 9.423 ± | 6.208 ± |
| | 0.664 | 0.372 | 0.793 | 0.499 | 0.969 | 0.858 | 0.932 | 0.483 | 1.885 | 1.428 |
| Heart | 27.688 ± | 25.695 ± | 15.685 ± | 13.323 ± | 12.088 ± | 10.25 ± | 11.740 ± | 9.915 ± | 8.140 ± | 7.463 ± |
| | 1.942 | 0.934 | 1.223 | 1.133 | 0.883 | 1.335 | 1.553 | 0.171 | 0.598 | 0.768 |
| Kidneys | 11.430 ± | 12.100 ± | 7.345 ± | 6.783 ± | 6.418 ± | 5.565 ± | 6.475 ± | 5.568 ± | 5.643 ± | 4.815 ± |
| | 0.387 | 0.872 | 0.322 | 0.811 | 0.761 | 0.680 | 0.493 | 0.550 | 0.222 | 0.450 |
| Spleen | 15.263 ± | 15.860 ± | 14.135 ± | 11.265 ± | 13.675 ± | 9.388 ± | 13.655 ± | 9.920 ± | 15.105 ± | 10.303 ± |
| | 2.166 | 0.974 | 2.010 | 1.706 | 2.195 | 1.389 | 3.606 | 1.414 | 2.959 | 1.102 |
| Bladder | 6.045 ± | 9.688 ± | 1.653 ± | 1.820 ± | 1.443 ± | 1.403 ± | 1.318 ± | 1.710 ± | 1.115 ± | 1.293 ± |
| | 3.910 | 4.991 | 0.107 | 0.283 | 0.205 | 0.160 | 0.108 | 0.346 | 0.224 | 0.430 |
| Muscle | 1.608 ± | 1.435 ± | 2.608 ± | 1.780 ± | 2.368 ± | 1.955 ± | 2.408 ± | 2.148 ± | 2.095 ± | 1.918 ± |
| | 0.182 | 0.198 | 0.196 | 0.137 | 0.259 | 0.339 | 0.181 | 0.176 | 0.168 | 0.144 |
| Stomach | 3.238 ± | 3.978 ± | 2.875 ± | 3.073 ± | 2.478 ± | 2.238 ± | 2.260 ± | 2.233 ± | 2.380 ± | 1.665 ± |
| | 1.063 | 0.632 | 0.921 | 0.566 | 0.296 | 0.487 | 0.306 | 0.491 | 0.405 | 0.148 |
| Bone | 3.683 ± | 3.023 ± | 3.310 ± | 2.738 ± | 4.600 ± | 3.493 ± | 4.850 ± | 4.658 ± | 8.993 ± | 7.635 ± |
| | 1.418 | 0.244 | 0.330 | 0.171 | 0.511 | 0.716 | 1.292 | 1.399 | 1.057 | 0.872 |

Estimated human mean residence time (MRT) values are provided in Table 22 for each of the source organs. MRT in the remainder of the body was obtained by subtracting the sum of all source organ residence times from the reciprocal of the ⁸⁹Zr decay constant (Huang et al., Biodistribution, toxicity and radiation dosimetry studies of the serotonin transporter radioligand 4-[18F]-ADAM in rats and monkeys. Eur J Nucl Med Mol Imaging, 2010; 37: 545-555). This represents a conservative estimation of the cumulative tissue radioactivity.

TABLE 22
Human Mean Residence Times (h)

| Organ/ | Physical Decay¹ | | Biexponential Fit² | |
|---|---|---|---|---|
| Tissue | Female | Male | Female | Male |
| Brain | 0.398 | 0.364 | 0.372 | 0.344 |
| Stomach | 0.511 | 0.476 | 0.492 | 0.480 |
| Contents | | | | |
| Heart | 2.433 | 2.279 | 2.290 | 2.154 |
| Contents | | | | |
| Kidneys | 0.868 | 0.818 | 0.832 | 0.794 |
| Liver | 5.902 | 5.919 | 8.240 | 5.938 |
| Lungs | 2.508 | 2.772 | 2.411 | 2.642 |
| Muscle | 17.635 | 23.677 | 13.348 | 17.182 |
| Red Marrow | 2.777 | 2.024 | 2.613 | 1.913 |
| Spleen | 0.996 | 0.871 | 1.053 | 0.910 |
| Bladder | 0.299 | 0.491 | 0.315 | 0.405 |
| Contents | | | | |
| Remainder | 78.794 | 73.430 | 81.157 | 80.361 |
| of Body | | | | |

¹Mean residence time calculated assuming only physical decay following day 6 time point
²Mean residence time calculated from a biexponential fit of the data

The estimated absorbed tissue doses for all target organs for the OLINDA/EXM 1.1 adult male and adult female phantoms are provided in Table 23. The effective dose, defined by the International Commission on Radiological Protection (ICRP) (International Commission on Radiological Protection. 1990 Recommendations of the International Commission on Radiological Protection. ICRP Publication 60, Pergamon Press, New York, 1991) is a quantity that is calculated by multiplying the absorbed dose for a given organ by a stochastic risk weighting factor and adding the weighted doses together. Estimated effective doses are provided at the end of Table 23. These values represent a conservative estimation of radioactive absorbed doses.

TABLE 23
Estimated Human Tissue Absorbed Doses and Effective Dose

| | Physical Decay¹ | | Biexponential Fit² | |
|---|---|---|---|---|
| | Adult Male | Adult Female | Adult Male | Adult Female |
| Organ/Tissue | (mSv/MBq) | (mSv/MBq) | (mSv/MBq) | (mSv/MBq) |
| Adrenals | 0.561 | 0.702 | 0.567 | 0.726 |
| Brain | 0.179 | 0.237 | 0.182 | 0.234 |
| Breasts | 0.366 | 0.459 | 0.379 | 0.466 |
| Gallbladder | 0.601 | 0.692 | 0.610 | 0.751 |
| Wall | | | | |
| LLI Wall | 0.519 | 0.652 | 0.530 | 0.651 |
| Small Intestine | 0.563 | 0.600 | 0.582 | 0.605 |
| Stomach Wall | 0.575 | 0.714 | 0.584 | 0.718 |
| ULI Wall | 0.553 | 0.685 | 0.571 | 0.700 |
| Heart Wall | 0.789 | 0.973 | 0.781 | 0.964 |
| Kidney | 0.650 | 0.773 | 0.641 | 0.774 |
| Liver | 0.764 | 0.974 | 0.764 | 1.220 |
| Lungs | 0.575 | 0.705 | 0.561 | 0.700 |
| Muscle | 0.396 | 0.481 | 0.381 | 0.464 |
| Ovaries | 0.533 | 0.645 | 0.542 | 0.642 |
| Pancreas | 0.597 | 0.743 | 0.606 | 0.765 |
| Red Marrow | 0.480 | 0.591 | 0.483 | 0.587 |
| Osteogenic | 0.604 | 0.777 | 0.625 | 0.779 |
| Cells | | | | |
| Skin | 0.291 | 0.373 | 0.297 | 0.374 |
| Spleen | 0.856 | 1.120 | 0.876 | 1.160 |
| Testes | 0.399 | NA | 0.407 | NA |
| Thymus | 0.481 | 0.605 | 0.484 | 0.601 |
| Thyroid | 0.417 | 0.484 | 0.423 | 0.480 |
| Urinary | 0.580 | 0.496 | 0.559 | 0.494 |
| Bladder Wall | | | | |
| Uterus | 0.545 | 0.638 | 0.554 | 0.636 |
| Total Body | 0.440 | 0.550 | 0.440 | 0.554 |
| Effective Dose | 0.513 | 0.622 | 0.516 | 0.625 |

¹Absorbed doses calculated from MRT assuming only physicadecay following day 6 time point
2Absorbed doses calculated from MRT with a biexponential fit of the data
Abbreviations: LLI = lower large intestine, ULI = upper large intestine, NA = not applicable

The estimated human tissue absorbed doses and effective human dose (Table 23) from the physical decay and the biexponential fit methods were similar. The physical decay method was selected to produce the final set of estimated human tissue absorbed doses and effective dose due to the apparent MAHA response in this murine model. Therefore, the effective human dose for ⁸⁹Zr-DFO-anti-PD-L1 antibody conjugate was estimated to be 0.513 mSv/MBq in the adult male and 0.622 mSv/MBq in the adult female. The organs predicted to have the highest absorbed dose in humans are the spleen and liver. The estimated absorbed dose in the spleen was 0.856 mSv/MBq in the adult male and 1.12 mSv/MBq in the adult female. The estimated absorbed dose in the liver was 0.764 mSv/MBq in the adult male and 0.974 mSv/MBq in the adult female.

### Example 10: ImmunoPET Imaging of PD-L1 in Tumors Using an ⁸⁹Zr-DFO-Anti-PD-L1 Antibody Conjugate in Patients with Advanced Thoracic Malignancies

The primary objective of this study is to determine the safety and tolerability of ⁸⁹Zr-DFO-anti-PD-L1 antibody conjugate, in which the anti-PD-L1 antibody used in the radiolabeled conjugate is H4H8314N. The secondary objectives of the study are:
Study part A only: To establish adequate mass dose of ⁸⁹Zr-DFO-anti-PD-L1 antibody conjugate and optimal post-infusion imaging time, as assessed by imaging and blood draw after tracer infusion.
Study part B only: To establish test/re-test reliability of PET measures as assessed on two separate tracer infusions at optimal mass dose and imaging time point as determined in Part A.
To characterize the pharmacokinetic (PK) profile of ⁸⁹Zr-DFO-anti-PD-L1 antibody conjugate based on tracer plasma activity concentration.

This is an open label, 2-part study designed to evaluate the safety and tolerability of ⁸⁹Zr-DFO-anti-PD-L1. Study Part A will establish an adequate mass dose and activity dose of ⁸⁹Zr-DFO-anti-PD-L1 and an optimal post-infusion imaging time. Test/re-test variability of ⁸⁹Zr-DFO-anti-PD-L1 will be evaluated in Part B.

All patients will undergo screening procedures. Patients who meet the eligibility criteria will undergo ¹⁸F-fluorodeoxyglucose (¹⁸F-FDG) PET/computed tomography (CT) and diagnostic CT scans to assess lesion viability, location, and dimensions. These scans will not be required if adequate quality images are available that were acquired within 28 days of the expected first dose of ⁸⁹Zr-DFO-anti-PD-L1.

### Part A

Three sequential dose cohorts are planned to be treated open-label with ⁸⁹Zr-DFO-anti-PD-L1 at 5 mg, 10 mg, or 20 mg.

After infusion with ⁸⁹Zr-DFO-anti-PD-L1, patients will undergo ⁸⁹Zr-DFO-anti-PD-L1 PET/CT scans on day 1, day 4±1 and day 7±1. Additional imaging may be performed up to day 10. Patients will undergo safety assessments and provide samples for hematology, chemistry, immune safety assays, pharmacokinetics, anti-drug antibody analysis, and biomarker analysis.

Patients will continue to undergo safety evaluations, including physical examination, vital signs, and documentation of Adverse Events (AEs), up to day 21 after the infusion of the ⁸⁹Zr-DFO-anti-PD-L1 tracer.

Dose escalation decisions to identify an adequate dose will be informed by safety and tolerability data and by evaluation of immune-positron emission tomography (iPET) positivity and tracer plasma activity concentration, as described below.

### Dose Cohorts in Part A

Up to 3 ascending mass dose cohorts are planned. For each mass dose cohort, an initial 2 patients will be dosed, with at a minimum 48-hour interval between the dosing of each patient. Upon completion of the day 7±1 day PET/CT scan for the second patient at a given mass dose, all available imaging, tracer plasma activity concentration, clinical dosimetry, and safety data will be reviewed. Based upon this review, a decision will be made to:
Expand the cohort 6 patients, if there is tumor uptake positivity/tumor localization in at least 1 patient, as defined by a tumor-to-blood ratio>1
Ascend to the next mass dose cohort if there is inadequate tumor uptake and plasma tracer activity concentration, with adequate defined by blood standardized uptake value (SUV) range of 1 to 5 at the optimum imaging time point
Proceed with the next mass dose cohort at a lower mass dose, based on inadequate tumor uptake and adequate plasma tracer activity concentration.

If tumor localization is inadequate in at least 2 patients at all three proposed mass dose levels, and this is determined to be due to low image signal-to-noise, the activity dose will be increased up to a maximum of 185 MBq for further expansion of previously tested mass dose cohorts.

### Part B

Study Part B will begin once an adequate ⁸⁹Zr-DFO-anti-PD-L1 dose and an optimal imaging time have been determined in Part A. On day 1 of Part B, patients will receive the tracer mass dose. Subsequent to receiving the tracer, patients will undergo a scan at the optimal time as identified in Part A. Patients in Part B will receive a second tracer dose and scan after an inter-dose interval of 14 to 28 days. The actual timing of the second tracer dose after the interval will be determined based on results from Part A.

Patients will undergo safety assessments, including physical examination, vital signs, and documentation of adverse events (AEs) during and after visits where ⁸⁹Zr-DFO-anti-PD-L1 tracer is administered. During these visits, patients will provide samples for PK, hematology, chemistry, and immune safety assays.

For both Part A and Part B, patients will continue to undergo safety evaluations, including physical examination, vital signs, and documentation of AEs, up to 21 days after the last infusion of the ⁸⁹Zr-DFO-anti-PD-L1 tracer.

### Study Duration

For Part A, patients will have a screening period of up to 28 days (4 weeks) and a follow-up period of up to 21 days (approximately 3 weeks) after infusion of the tracer dose. The duration of study Part A is approximately 7 weeks, including the screening period.

For Part B, patients will have a screening period of up to 28 days (4 weeks), an inter-infusion interval of up to 28 days (4 weeks), and a 21-day (3 week) safety follow-up period that includes the second scan period. The total duration of the study for each patient will be up to 11 weeks, including the screening period.

The end of study for this study is defined as the last visit of the last patient.

For study Part A, 3 sequential dose levels of up to 6 patients each are planned per cohort, with 3 cohorts planned, for a total of up to 18 patients. For study Part B, up to 10 patients will be enrolled. Enrollment of a maximum of 28 patients in a single study site is planned for the entire study.

### Patient Target Population

The target population will consist of patients 18 years of age or older with advanced thoracic malignancies and PD-L 1 IHC score on a diagnostic or subsequent biopsy of ≥1% (positive PD-L1 IHC score by 22C3 PharmDx assay, Dako North America Inc.).
For Part A, the thoracic malignancies will be limited to NSCLC, gastro-esophageal junction adenocarcinoma, and gastric cancer, with PD-L1 score of ≥1% by IHC.
For Part B, all patients with advanced thoracic malignancies and a PD-L1 score of ≥1% by IHC will be eligible. Patients must also have stable disease as per RECIST 1.1 between the two most recent imaging studies.

All patients requiring therapy should be on standard of care therapy.

### Treatment

⁸⁹Zr-DFO-anti-PD-L1, a radioimmunoconjugate formed by covalently conjugating bifunctional chelator (p-SCN-Bn-DFO) to H4H8314N (anti-PD-L1 monoclonal antibody) and radiolabeling this compound with ⁸⁹Zr. ⁸⁹Zr-DFO-anti-PD-L1 is supplied in an aqueous buffered vehicle.

For Part A, ⁸⁹Zr-DFO-anti-PD-L1 will be administered IV on day 1 (baseline). For Part B, ⁸⁹Zr-DFO-anti-PD-L1 will be administered IV on day 1 and day 7±3. Actual timing of the second dose in Part B will be determined from results in Part A.

The ⁸⁹Zr-DFO-anti-PD-L1 tracer will be administered at a dose level well below the estimated cumulative exposure levels in humans based on PK models and lower than the levels at which currently available anti-PD-1 agents are used for anti-cancer treatment. This study will exclude patients who are currently treated with anti-PD-L1 to avoid competition for target.

### Endpoints

The primary endpoint in the study is the incidence and severity of Treatment-emergent adverse events (TEAEs) through day 21 of the last dose of tracer infusion in patients with thoracic malignancies dosed with ⁸⁹Zr-DFO-anti-PD-L1.

For Part A only, the study will establish an adequate mass dose and activity dose of ⁸⁹Zr-DFO-anti-PD-L1 and optimal post-infusion imaging time, and the following will be determined via blood drawing and imaging at day 1, 4, and 7 after tracer infusion:
Standardized uptake value of ⁸⁹Zr-DFO-anti-PD-L1 in the blood pool, with subsequent calculation of tumor-to-blood ratios at the time of imaging
Clinical dosimetry based on the absorbed dose and effective tissue radiation, as calculated from PET image acquisition data and tracer activity concentration in blood
Standardized Uptake Values (SUVs) across the tumor regions of interest (ROIs)
Maximal SUVs (SUVmax) within tumor ROIs
Plasma tracer activity concentration, expressed as SUV, with calculation of area under the curve through day 7 (AUC₀₋₇)

For Part B only, the study will establish the test/re-test reliability of ⁸⁹Zr-DFO-anti-PD-L1 PET measures, and the following will be determined from measures of 2 separate tracer infusions at an adequate mass dose and optimal imaging time points, as determined from Part A:
Blood pool SUV with subsequent calculation of tumor-to-blood ratio
SUVs across the tumor ROIs
SUVmax within the tumor ROIs
Biodistribution of ⁸⁹Zr-DFO-anti-PD-L1

The resulting data will be indicative of the safety and tolerability of ⁸⁹Zr-DFO-anti-PD-L1 in humans.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

TABLE 24
Informal Sequence Listing

| SEQ. | | |
|---|---|---|
| ID. NO. | SEQUENCE | DESCRIPTION |
| 1 | gaggtgcagc tggtggagtc tgggggaggc ttggtccagc ctggggggtc | DNA |
| | cctgagactc tcctgtgcag cctctggatt cacctttagt aggttttgga | nucleotide |
| | tgagctgggt ccgccaggct ccagggaagg ggctggagtg ggtggccaac | sequence |
| | ataaaccaag atggaactga gaaatactat gtggactctg tgaagggccg | |
| | attcaccato tccagagaca acgccaagaa ctcactgtat ctgcaaatga | |
| | acagcctgag agccggggac acggctgtgt attactgtgc gaatacgtat | |
| | tacgattttt ggagtggtca ctttgactac tggggccagg gaaccctggt | |
| | caccgtctcc tca | |
| 2 | EVQLVESGGG LVQPGGSLRL SCAASGFTFS RFWMSWVRQA PGKGLEWVAN | AA amino acid |
| | INQDGTEKYY VDSVKGRFTI SRDNAKNSLY LQMNSLRAGD TAVYYCANTY | sequence |
| | YDFWSGHFDY WGQGTLVTVS S | |
| 3 | ggattcacct ttagtaggtt ttgg | DNA |
| | | nucleotide |
| | | sequence |
| 4 | GFTFSRFW | AA amino acid |
| | | sequence |
| 5 | ataaaccaag atggaactga gaaa | DNA |
| | | nucleotide |
| | | sequence |
| 6 | INQDGTEK | AA amino acid |
| | | sequence |
| 7 | gcgaatacgt attacgattt ttggagtggt cactttgact ac | DNA |
| | | nucleotide |
| | | sequence |
| 8 | ANTYYDFWSG HFDY | AA amino acid |
| | | sequence |
| 9 | gacatccaga tgacccagtc tccttccacc ctgtctgcat ctgtaggaga | DNA |
| | cagagtcacc atcacttgtc gggccagtca gagtattagt aattggttgg | nucleotide |
| | cctggtatca gcagaaacca gggaaagccc ctaagctcct gatctataag | sequence |
| | gcgtctagtt tagaaagtgg ggtcccatca aggttcagcg gcagtggatc | |
| | tgggacagaa ttcactctca ccatcagcag cctgcagcct gatgattttg | |
| | caacttatta ctgccaacag tatcatagtt attcgtacac ttttggccag | |
| | gggaccaagc tggagatcaa a | |
| 10 | DIQMTQSPST LSASVGDRVT ITCRASQSIS NWLAWYQQKP GKAPKLLIYK | AA amino acid |
| | ASSLESGVPS RFSGSGSGTE FTLTISSLQP DDFATYYCQQ YHSYSYTFGQ | sequence |
| | GTKLEIK | |
| 11 | cagagtatta gtaattgg | DNA |
| | | nucleotide |
| | | sequence |
| 12 | QSISNW | AA amino acid |
| | | sequence |
| 13 | aaggcgtct | DNA |
| | | nucleotide |
| | | sequence |
| 14 | KAS | AA amino acid |
| | | sequence |
| 15 | caacagtatc atagttattc gtacact | DNA |
| | | nucleotide |
| | | sequence |
| 16 | QQYHSYSYT | AA amino acid |
| | | sequence |
| 17 | caggagcacc tggtggagtc tgggggaggc gtggtccagc ctgggaggtc | DNA |
| | cctgagactc toctgtgaag cgtctggatt caccttcagt aactttggca | nucleotide |
| | tgcactgggt ccgccaggct ccaggcaagg ggctggagtg ggtggcagct | sequence |
| | ttatggtctg atggaagtaa taaatactat gcagactccg tgaagggtcg | |
| | agtcaccatc tccagagaca attccaagaa cacactgtat ctgcaaatga | |
| | acagcctgag agccgaggac acggctgtct attactgtgc gagagggaga | |
| | ggagcccccg gtattccgat ttttgggtac tggggccagg gaaccctggt | |
| | caccgtctcc tca | |
| 18 | QEHLVESGGG VVQPGRSLRL SCEASGFTFS NFGMHWVRQA PGKGLEWVAA | AA amino acid |
| | LWSDGSNKYY ADSVKGRVTI SRDNSKNTLY LQMNSLRAED TAVYYCARGR | sequence |
| | GAPGIPIFGY WGQGTLVTVS S | |
| 19 | ggattcacct tcagtaactt tggc | DNA |
| | | nucleotide |
| | | sequence |
| 20 | GFTFSNFG | AA amino acid |
| | | sequence |
| 21 | ttatggtctg atggaagtaa taaa | DNA |
| | | nucleotide |
| | | sequence |
| 22 | LWSDGSNK | AA amino acid |
| | | sequence |
| 23 | gcgagaggga gaggagcccc cggtattccg atttttgggt ac | DNA |
| | | nucleotide |
| | | sequence |
| 24 | ARGRGAPGIP IFGY | AA amino acid |
| | | sequence |
| 25 | gacatccaga tgacccagtc tccatcctcc ctgtctgcat ctgttggaga | DNA |
| | cagagtcacc atcacttgcc gggcaagtca gggcattaga aatgatttag | nucleotide |
| | gctggtatca gcagaaacca gggaaagccc ctaagcgcct gatctatact | sequence |
| | gcatccagtt tgcaaagtgg ggtcccatca agattcagcg gcagtggatc | |
| | tgggacagaa ttcactctca caatcagcag cctacagcct gaagattttg | |
| | caacttatta ctgtctacaa cataatagtt accctctcac attcggcgga | |
| | gggaccaagg tggcgatcaa a | |
| 26 | DIQMTQSPSS LSASVGDRVT ITCRASQGIR NDLGWYQQKP GKAPKRLIYT | AA amino acid |
| | ASSLQSGVPS RFSGSGSGTE FTLTISSLQP EDFATYYCLQ HNSYPLTFGG | sequence |
| | GTKVAIK | |
| 27 | cagggcatta gaaatgat | DNA |
| | | nucleotide |
| | | sequence |
| 28 | QGIRND | AA amino acid |
| | | sequence |
| 29 | actgcatcc | DNA |
| | | nucleotide |
| | | sequence |
| 30 | TAS | AA amino acid |
| | | sequence |
| 31 | ctacaacata atagttaccc tctcaca | DNA |
| | | nucleotide |
| | | sequence |
| 32 | LQHNSYPLT | AA amino acid |
| | | sequence |
| 33 | gaggtgcagc tggtggagtc tgggggaggc ttggtaaagc ctggggggtc | DNA |
| | ccttagactc tcctgtgcag cctctggatt cactttcagt aacgcctgga | nucleotide |
| | tgagctgggt ccgccaggct ccagggaagg ggctggagtg ggttggccgt | sequence |
| | attaaaagga aaactgatgg tgggacaaca gactacgctg cacccgtgaa | |
| | aggcagattc accatctcaa gagatgattc aaaaaatacg ctgcatctgc | |
| | aaatgaacag cctgaaaacc gaggacacag ccgtgtatta ctgtaccaca | |
| | gatgatattg tagttgtacc agctgttatg agggaatact acttcggtat | |
| | ggacgtctgg ggccaaggga ccacggtcac cgtctcctca | |
| 34 | EVQLVESGGG LVKPGGSLRL SCAASGFTFS NAWMSWVRQA PGKGLEWVGR | AA amino acid |
| | IKRKTDGGTT DYAAPVKGRF TISRDDSKNT LHLQMNSLKT EDTAVYYCTT | sequence |
| | DDIVVVPAVM REYYFGMDVW GQGTTVTVSS | |
| 35 | ggattcactt tcagtaacgc ctgg | DNA |
| | | nucleotide |
| | | sequence |
| 36 | GFTFSNAW | AA amino acid |
| | | sequence |
| 37 | attaaaagga aaactgatgg tgggacaaca | DNA |
| | | nucleotide |
| | | sequence |
| 38 | IKRKTDGGTT | AA amino acid |
| | | sequence |
| 39 | accacagatg atattgtagt tgtaccagct gttatgaggg aatactactt | DNA |
| | cggtatggac gtc | nucleotide |
| | | sequence |
| 40 | TTDDIVVVPA VMREYYFGMD V | AA amino acid |
| | | sequence |
| 41 | gacatccaga tgacccagtc tccatcctcc ctgtctgcat ctgtaggaga | DNA |
| | cagagtcacc atcacttgcc ggacaagtca gggcattaga aatgatttag | nucleotide |
| | gctggtatca gcagaaacca gggaaagccc ctaagcgcct gatctatgct | sequence |
| | gcatccagtt tgcaaagtgg ggtcccatca aggttcagcg gcagtggatc | |
| | tgggacagaa ttcactctca caatcagcag cctgcagcct gaagattttg | |
| | caacttatta ctgtctacag cataataatt acccgtacac ttttggccag | |
| | gggaccaagc tggagatcaa a | |
| 42 | DIQMTQSPSS LSASVGDRVT ITCRTSQGIR NDLGWYQQKP GKAPKRLIYA | AA amino acid |
| | ASSLQSGVPS RFSGSGSGTE FTLTISSLQP EDFATYYCLQ HNNYPYTFGQ | sequence |
| | GTKLEIK | |
| 43 | cagggcatta gaaatgat | DNA |
| | | nucleotide |
| | | sequence |
| 44 | QGIRND | AA amino acid |
| | | sequence |
| 45 | gctgcatcc | DNA |
| | | nucleotide |
| | | sequence |
| 46 | AAS | AA amino acid |
| | | sequence |
| 47 | ctacagcata ataattacco gtacact | DNA |
| | | nucleotide |
| | | sequence |
| 48 | LQHNNYPYT | AA amino acid |
| | | sequence |
| 49 | caggtgcaat tggtgcagtc tggggcggag gtgaagaagc ctggggcctc | DNA |
| | agtgcaggtc toctgcaagg cttctggata ctccttcacc ggctactata | nucleotide |
| | tacactgggt gcgacaggcc cctggacaag gacttgagtg gatgggatgg | sequence |
| | atcaacccta acagtggcac caaaaagtat gcacacaagt ttcagggcag | |
| | ggtcaccatg accagggaca cgtccatoga cacagcctac atgattttga | |
| | gcagtctgat atccgacgac acggccgtgt attactgtgc gagagatgag | |
| | gactggaact ttgggagctg gttcgactcc tggggccagg gaaccctggt | |
| | caccgtctcc tca | |
| 50 | QVQLVQSGAE VKKPGASVQV SCKASGYSFT GYYIHWVRQA PGQGLEWMGW | AA amino acid |
| | INPNSGTKKY AHKFQGRVTM TRDTSIDTAY MILSSLISDD TAVYYCARDE | sequence |
| | DWNFGSWFDS WGQGTLVTVS S | |
| 51 | ggatactcct tcaccggcta ctat | DNA |
| | | nucleotide |
| | | sequence |
| 52 | GYSFTGYY | AA amino acid |
| | | sequence |
| 53 | atcaacccta acagtggcac caaa | DNA |
| | | nucleotide |
| | | sequence |
| 54 | INPNSGTK | AA amino acid |
| | | sequence |
| 55 | gcgagagatg aggactggaa ctttgggagc tggttcgact cc | DNA |
| | | nucleotide |
| | | sequence |
| 56 | ARDEDWNFGS WFDS | AA amino acid |
| | | sequence |
| 57 | gatattgtga tgacccagac tccactctcc tcacctgtca cccttggaca | DNA |
| | gccggcctcc atctcctgca ggtctagtca aaccctcgta cacggtgatg | nucleotide |
| | gaaacacgta cttgagttgg attcagcaga ggccaggcca gcctccgaga | sequence |
| | ctcctcattt ataaggtttc taatcagttc totggggtcc cagacagatt | |
| | cagtggcagt ggggcaggga cagatttcac actgaaaatc agcagggtgg | |
| | aagctgagga tgtcgggctt tatttctgca tgcaagctac acattttccg | |
| | atcaccttcg gccaagggac acgactggag attaaa | |
| 58 | DIVMTQTPLS SPVTLGQPAS ISCRSSQTLV HGDGNTYLSW IQQRPGQPPR | AA amino acid |
| | LLIYKVSNQF SGVPDRFSGS GAGTDFTLKI SRVEAEDVGL YFCMQATHFP | sequence |
| | ITFGQGTRLE IK | |
| 59 | caaaccctcg tacacggtga tggaaacacg tac | DNA |
| | | nucleotide |
| | | sequence |
| 60 | QTLVHGDGNT Y | AA amino acid |
| | | sequence |
| 61 | aaggtttct | DNA |
| | | nucleotide |
| | | sequence |
| 62 | KVS | AA amino acid |
| | | sequence |
| 63 | atgcaagcta cacattttcc gatcacc | DNA |
| | | nucleotide |
| | | sequence |
| 64 | MQATHFPIT | AA amino acid |
| | | sequence |
| 65 | caggtacacc tggtgcagtc tggggctgag gtgaagaagc ctggggcctc | DNA |
| | agtgaaggtc tcctgcaagg cttctggata caccttcacc ggctactata | nucleotide |
| | tacattgggt gcgacaggcc cctggacacg ggcttgagtg gatgggatgg | sequence |
| | ctcaacccta atactggtac cacaaagtat atacagaact ttcagggcag | |
| | ggtcaccatg accagggaca cgtccagcag cacagcctac atggagctga | |
| | ccaggctgag atctgacgac acggccgtgt attactgtgc gagagatgag | |
| | gactggaatt atgggagctg gttcgacacc tggggccagg gaaccctggt | |
| | cacagtctcc tca | |
| 66 | QVHLVQSGAE VKKPGASVKV SCKASGYTFT GYYIHWVRQA PGHGLEWMGW | AA amino acid |
| | LNPNTGTTKY IQNFQGRVTM TRDTSSSTAY MELTRLRSDD TAVYYCARDE | sequence |
| | DWNYGSWFDT WGQGTLVTVS S | |
| 67 | ggatacacct tcacoggcta ctat | DNA |
| | | nucleotide |
| | | sequence |
| 68 | GYTFTGYY | AA amino acid |
| | | sequence |
| 69 | ctcaacccta atactggtac caca | DNA |
| | | nucleotide |
| | | sequence |
| 70 | LNPNTGTT | AA amino acid |
| | | sequence |
| 71 | gcgagagatg aggactggaa ttatgggagc tggttcgaca cc | DNA |
| | | nucleotide |
| | | sequence |
| 72 | ARDEDWNYGS WFDT | AA amino acid |
| | | sequence |
| 73 | gatattgtaa tgacccagac tccactctcc tcacctgtca cccttggaca | DNA |
| | gccggcctcc atctcctgca ggtctagtcc aagcctcgta cacagtgatg | nucleotide |
| | gaaacaccta cttgagttgg cttcagcaga ggccaggcca gcctccaaga | sequence |
| | ctcctaattt ataagatttc taaccgatto totggggtcc cagacagatt | |
| | cagtggcagt ggggcaggga cagatttcac gctgaaaatc agcagggtgg | |
| | aagctgagga tgtcggggtt tattactgca tgcaagctac acattttccg | |
| | atcaccttcg gccaagggac acgactggag attaga | |
| 74 | DIVMTQTPLS SPVTLGQPAS ISCRSSPSLV HSDGNTYLSW LQQRPGQPPR | AA amino acid |
| | LLIYKISNRF SGVPDRFSGS GAGTDFTLKI SRVEAEDVGV YYCMQATHFP | sequence |
| | ITFGQGTRLE IR | |
| 75 | ccaagcctcg tacacagtga tggaaacacc tac | DNA |
| | | nucleotide |
| | | sequence |
| 76 | PSLVHSDGNT Y | AA amino acid |
| | | sequence |
| 77 | aagatttct | DNA |
| | | nucleotide |
| | | sequence |
| 78 | KIS | AA amino acid |
| | | sequence |
| 79 | atgcaagcta cacattttcc gatcacc | DNA |
| | | nucleotide |
| | | sequence |
| 80 | MQATHFPIT | AA amino acid |
| | | sequence |
| 81 | gaggtgcagc tggtggaatc tgggggaggt gtggtgcggc ctggggggtc | DNA |
| | cctgagactc tcctgtgcag cctctggatt cacttttgat gattatggca | nucleotide |
| | tgacctgggt ccgccaagct ccagggaggg gcctggaatg ggtctctggt | sequence |
| | attcattggc atggtaaacg cacaggttat gcagactctg tgaagggccg | |
| | attcaccata tccagagaca acgccaagaa atccctgtat ctgcaaatga | |
| | acagtctgaa aggcgaggac acggccttgt atcattgtgt gaggggggga | |
| | atgagtacag gggactggtt cgacccctgg ggccagggaa ccctggtcat | |
| | cgtctcctca | |
| 82 | EVQLVESGGG VVRPGGSLRL SCAASGFTFD DYGMTWVRQA PGRGLEWVSG | AA amino acid |
| | IHWHGKRTGY ADSVKGRFTI SRDNAKKSLY LQMNSLKGED TALYHCVRGG | sequence |
| | MSTGDWFDPW GQGTLVIVSS | |
| 83 | ggattcactt ttgatgatta tggc | DNA |
| | | nucleotide |
| | | sequence |
| 84 | GFTFDDYG | AA amino acid |
| | | sequence |
| 85 | attcattggc atggtaaacg caca | DNA |
| | | nucleotide |
| | | sequence |
| 86 | IHWHGKRT | AA amino acid |
| | | sequence |
| 87 | gtgagggggg gaatgagtac aggggactgg ttcgacccc | DNA |
| | | nucleotide |
| | | sequence |
| 88 | VRGGMSTGDW FDP | AA amino acid |
| | | sequence |
| 89 | gacatccaga tgacccagtc tocatcctcc ctgtctgcat ctctaggaga | DNA |
| | cagagtcacc atcacttgcc gggcaagtca gagcattaac agttatttaa | nucleotide |
| | attggtatca gcagaaacca gggaaagccc ctaaactcct gatctatgtt | sequence |
| | gcatccagtt tgcaaagtgg ggtcccatca aggttcagtg gcagtggatc | |
| | tgggacagaa ttcactctca ccatcagcaa totgcaacct gaagattttg | |
| | caacttacta ctgtcaacag agttacagta cccctccgat caccttcggc | |
| | caagggacac gactggagat taaa | |
| 90 | DIQMTQSPSS LSASLGDRVT ITCRASQSIN SYLNWYQQKP GKAPKLLIYV | AA amino acid |
| | ASSLQSGVPS RFSGSGSGTE FTLTISNLQP EDFATYYCQQ SYSTPPITFG | sequence |
| | QGTRLEIK | |
| 91 | cagagcatta acagttat | DNA |
| | | nucleotide |
| | | sequence |
| 92 | QSINSY | AA amino acid |
| | | sequence |
| 93 | gttgcatcc | DNA |
| | | nucleotide |
| | | sequence |
| 94 | VAS | AA amino acid |
| | | sequence |
| 95 | caacagagtt acagtacccc tccgatcaco | DNA |
| | | nucleotide |
| | | sequence |
| 96 | QQSYSTPPIT | AA amino acid |
| | | sequence |
| 97 | gaggtgcagc tggtggagtc tgggggaggt gtggtacggc cgggggggtc | DNA |
| | cctgagactc tcctgtgcag cctctggatt cacctttgat gattatggca | nucleotide |
| | tgacctgggt ccgccaagtt ccagggaagg ggctggagtg ggtctctggt | sequence |
| | attcattgga gtggtagaag cacaggttat gcagactctg tgaagggccg | |
| | attcaccatc tccagagaca acgccaagaa ctccctgtat ctgcaaatga | |
| | acagtctgag agccgaggac acggccttgt attactgtgc gaggggggga | |
| | atgagtacgg gggactggtt cgacccctgg ggccagggaa ccctggtcac | |
| | cgtctcctca | |
| 98 | EVQLVESGGG VVRPGGSLRL SCAASGFTFD DYGMTWVRQV PGKGLEWVSG | AA amino acid |
| | IHWSGRSTGY ADSVKGRFTI SRDNAKNSLY LQMNSLRAED TALYYCARGG | sequence |
| | MSTGDWFDPW GQGTLVTVSS | |
| 99 | ggattcacct ttgatgatta tggc | DNA |
| | | nucleotide |
| | | sequence |
| 100 | GFTFDDYG | AA amino acid |
| | | sequence |
| 101 | attcattgga gtggtagaag caca | DNA |
| | | nucleotide |
| | | sequence |
| 102 | IHWSGRST | AA amino acid |
| | | sequence |
| 103 | gcgagggggg gaatgagtac gggggactgg ttcgacccc | DNA |
| | | nucleotide |
| | | sequence |
| 104 | ARGGMSTGDW FDP | AA amino acid |
| | | sequence |
| 105 | gacatccaga tgacccagtc tccatcctcc ctgtctgcat ctgtaggaga | DNA |
| | cagagtcacc atcacttgcc gggcaagtca gagcattagc agctatttaa | nucleotide |
| | attggtatca gcagaaacca gggaaagccc ctaagctcct gatctatgtt | sequence |
| | gcatccagtt tgcaaagtgg ggtcccatca aggttcagtg gcagtggatc | |
| | tgggacagat ttcactctca ccatcagcag tctgcaacct gaagattttg | |
| | caacttacta ctgtcaacag agttacagta cccctccgat caccttcggc | |
| | caagggacac gactggagat taaa | |
| 106 | DIQMTQSPSS LSASVGDRVT ITCRASQSIS SYLNWYQQKP GKAPKLLIYV | AA amino acid |
| | ASSLQSGVPS RFSGSGSGTD FTLTISSLQP EDFATYYCQQ SYSTPPITFG | sequence |
| | QGTRLEIK | |
| 107 | cagagcatta gcagctat | DNA |
| | | nucleotide |
| | | sequence |
| 108 | QSISSY | AA amino acid |
| | | sequence |
| 109 | gttgcatcc | DNA |
| | | nucleotide |
| | | sequence |
| 110 | VAS | AA amino acid |
| | | sequence |
| 111 | caacagagtt acagtacccc tccgatcacc | DNA |
| | | nucleotide |
| | | sequence |
| 112 | QQSYSTPPIT | AA amino acid |
| | | sequence |
| 113 | gaggtgcagt tggtggagtc tggaggaggc ttggtccagc ctggggggtc | DNA |
| | cctaagactc toctgtgcag cctctgggtt caccgtoggt agtaactaca | nucleotide |
| | tgaactgggt ccgtcaggct ccagggaagg gactggagtg ggtctcagtt | sequence |
| | atttatagtg gtggtagtac atactacgca gattccgtga agggccgatt | |
| | caccatctcc agactcactt ccaagaacac actgtatott caaatgagca | |
| | gcctgagacc tgaggacacg gccgtgtatt attgtgcgag agggattagg | |
| | ggtctggacg tctggggcca agggaccacg gtcaccgtct cttca | |
| 114 | EVQLVESGGG LVQPGGSLRL SCAASGFTVG SNYMNWVRQA PGKGLEWVSV | AA amino acid |
| | IYSGGSTYYA DSVKGRFTIS RLTSKNTLYL QMSSLRPEDT AVYYCARGIR | sequence |
| | GLDVWGQGTT VTVSS | |
| 115 | gggttcaccg toggtagtaa ctac | DNA |
| | | nucleotide |
| | | sequence |
| 116 | GFTVGSNY | AA amino acid |
| | | sequence |
| 117 | atttatagtg gtggtagtac a | DNA |
| | | nucleotide |
| | | sequence |
| 118 | IYSGGST | AA amino acid |
| | | sequence |
| 119 | gcgagaggga ttaggggtct ggacgtc | DNA |
| | | nucleotide |
| | | sequence |
| 120 | ARGIRGLDV | AA amino acid |
| | | sequence |
| 121 | gacatccaga tgacccagtc tccatcctcc ctgtctgcat ctgtaggaga | DNA |
| | cagagtcacc atcacttgcc gggcaagtca gaccattaac atctatttaa | nucleotide |
| | attggtatca gcagaaacca gggagagccc ctaggctcct gatctatgct | sequence |
| | gcatccagtt tgcaaagtgg ggtcccatca aggttcagtg gcagtggatc | |
| | tgggacagat ttcactctca ccatcagcag tctgcaacct gaagattttg | |
| | caacttacta ctgtcaccag agttacagta cccctccgat caccttcggc | |
| | caagggacac gactggagat taaa | |
| 122 | DIQMTQSPSS LSASVGDRVT ITCRASQTIN IYLNWYQQKP GRAPRLLIYA | AA amino acid |
| | ASSLQSGVPS RFSGSGSGTD FTLTISSLQP EDFATYYCHQ SYSTPPITFG | sequence |
| | QGTRLEIK | |
| 123 | cagaccatta acatctat | DNA |
| | | nucleotide |
| | | sequence |
| 124 | QTINIY | AA amino acid |
| | | sequence |
| 125 | gctgcatcc | DNA |
| | | nucleotide |
| | | sequence |
| 126 | AAS | AA amino acid |
| | | sequence |
| 127 | caccagagtt acagtacccc tccgatcacc | DNA |
| | | nucleotide |
| | | sequence |
| 128 | HQSYSTPPIT | AA amino acid |
| | | sequence |
| 129 | gaggaacggt tggtggagtc tggaggagac ttggtccagc ctggggggtc | DNA |
| | cctgagactc tcctgtgcag cctctggcat caccgtcggt actaattata | nucleotide |
| | tgaactgggt ccgccaggct ccagggaagg gactggagtg ggtctcagtt | sequence |
| | atttctagcg gtggtaatac acactacgca gactccgtga agggccgatt | |
| | cattatgtcc agacaaactt ccaaaaacac gctgtatott cagatgaata | |
| | gcctggaaac tgaggacacg gccgtatatt attgtgcgag ggggatcaga | |
| | ggtttggacg tctggggcca agggaccatg gtcaccgtct cctca | |
| 130 | EERLVESGGD LVQPGGSLRL SCAASGITVG TNYMNWVRQA PGKGLEWVSV | AA amino acid |
| | ISSGGNTHYA DSVKGRFIMS RQTSKNTLYL QMNSLETEDT AVYYCARGIR | sequence |
| | GLDVWGQGTM VTVSS | |
| 131 | ggcatcaccg tcggtactaa ttat | DNA |
| | | nucleotide |
| | | sequence |
| 132 | GITVGTNY | AA amino acid |
| | | sequence |
| 133 | atttctagcg gtggtaatac a | DNA |
| | | nucleotide |
| | | sequence |
| 134 | ISSGGNT | AA amino acid |
| | | sequence |
| 135 | gcgaggggga tcagaggttt ggacgtc | DNA |
| | | nucleotide |
| | | sequence |
| 136 | ARGIRGLDV | AA amino acid |
| | | sequence |
| 137 | gacatccaga tgacccagtc tccatcctcc ctgtctgcat ctgtaggaga | DNA |
| | cagagtcacc atcacttgcc gggcaagtca gagcatgagc agctatttaa | nucleotide |
| | attggtatca gcagaaacca gggagagccc ctaagctcct gatctttgct | sequence |
| | gcatccagtt tgcaaagtgg ggtcccatca aggttcagtg gcagtggato | |
| | tgggacagat ttcactctca ccatcagcag tctgcaacct gaagattttg | |
| | caacttacta ctgtcaacag agttacagta cccctccgat caccttcggc | |
| | caagggacac gactggagat taaa | |
| 138 | DIQMTQSPSS LSASVGDRVT ITCRASQSMS SYLNWYQQKP GRAPKLLIFA | AA amino acid |
| | ASSLQSGVPS RFSGSGSGTD FTLTISSLQP EDFATYYCQQ SYSTPPITFG | sequence |
| | QGTRLEIK | |
| 139 | cagagcatga gcagctat | DNA |
| | | nucleotide |
| | | sequence |
| 140 | QSMSSY | AA amino acid |
| | | sequence |
| 141 | gctgcatcc | DNA |
| | | nucleotide |
| | | sequence |
| 142 | AAS | AA amino acid |
| | | sequence |
| 143 | caacagagtt acagtacccc tccgatcacc | DNA |
| | | nucleotide |
| | | sequence |
| 144 | QQSYSTPPIT | AA amino acid |
| | | sequence |
| 145 | caggtccagc tggtgcagtc tggggctgag gtgaagatgc ctgggtcctc | DNA |
| | ggtgagggtc tcctgcaagg cttctggagg catcttcagc agttctacta | nucleotide |
| | tcagttgggt gogacaggcc cctggacaag ggcttgaatg gatgggagag | sequence |
| | atcatccctg totttggtac agtaaactac gcacagaagt tocaggacag | |
| | agtcatattt accgcggacg aatctacgac tacagcctac atggagctga | |
| | gcagcctgaa atctggggac acggccgtat atttotgtgc gcgaaattgg | |
| | ggattaggct ctttttatat ctggggccaa gggacaatgg tcaccgtctc | |
| | ttca | |
| 146 | QVQLVQSGAE VKMPGSSVRV SCKASGGIFS SSTISWVRQA PGQGLEWMGE | AA amino acid |
| | IIPVFGTVNY AQKFQDRVIF TADESTTTAY MELSSLKSGD TAVYFCARNW | sequence |
| | GLGSFYIWGQ GTMVTVSS | |
| 147 | ggaggcatct tagcagttc tact | DNA |
| | | nucleotide |
| | | sequence |
| 148 | GGIFSSST | AA amino acid |
| | | sequence |
| 149 | atcatccctg tctttggtac agta | DNA |
| | | nucleotide |
| | | sequence |
| 150 | IIPVFGTV | AA amino acid |
| | | sequence |
| 151 | gcgcgaaatt ggggattagg ctctttttat atc | DNA |
| | | nucleotide |
| | | sequence |
| 152 | ARNWGLGSFY I | AA amino acid |
| | | sequence |
| 153 | gaaattgtgt tgacgcagtc tccaggcacc ctgtotttgt ctccagggga | DNA |
| | aagagccacc ctctcctgca gggccagtca gagttttaac ttcaactact | nucleotide |
| | tagcctggta ccagcagaaa cctggccagg ctcccagact cctcatctat | sequence |
| | ggtgcatcca gcagggccac tggcatccca gacaggttca gtggcagtgg | |
| | gtctgggaca gacttcactc tcaccatcaa caggctggag cctgaagatt | |
| | ttggagtgtt ttattgtcag cagtatgaaa gcgcaccttg gacgttcggc | |
| | caagggacca aggtggaaat caaa | |
| 154 | EIVLTQSPGT LSLSPGERAT LSCRASQSFN FNYLAWYQQK PGQAPRLLIY | AA amino acid |
| | GASSRATGIP DRFSGSGSGT DFTLTINRLE PEDFGVFYCQ QYESAPWTFG | sequence |
| | QGTKVEIK | |
| 155 | cagagtttta acttcaacta c | DNA |
| | | nucleotide |
| | | sequence |
| 156 | QSFNFNY | AA amino acid |
| | | sequence |
| 157 | ggtgcatcc | DNA |
| | | nucleotide |
| | | sequence |
| 158 | GAS | AA amino acid |
| | | sequence |
| 159 | cagcagtatg aaagcgcacc ttggacg | DNA |
| | | nucleotide |
| | | sequence |
| 160 | QQYESAPWT | AA amino acid |
| | | sequence |
| 161 | gaggtgcagc ttgtagagtc tgggggagac ttggtacatc ctggcaggtc | DNA |
| | cctgagactc tcctgtgcag cctctggttt cccctttgat gagtatgcca | nucleotide |
| | tgcactgggt ccggcaagtt ccagggaagg gcctggagtg ggtctcaggt | sequence |
| | attagttgga gtaataataa cataggctat gcggactctg tgaagggccg | |
| | attcaccato tccagagaca acgccaaaaa ctccctgtat ctacaaatga | |
| | acagtctgag acctgaggac acggcctttt attactgtgc aaaatctgga | |
| | atctttgact cctggggcca gggaaccctg gtcaccgtct cctca | |
| 162 | EVQLVESGGD LVHPGRSLRL SCAASGFPFD EYAMHWVRQV PGKGLEWVSG | AA amino acid |
| | ISWSNNNIGY ADSVKGRFTI SRDNAKNSLY LQMNSLRPED TAFYYCAKSG | sequence |
| | IFDSWGQGTL VTVSS | |
| 163 | ggtttcccct ttgatgagta tgcc | DNA |
| | | nucleotide |
| | | sequence |
| 164 | GFPFDEYA | AA amino acid |
| | | sequence |
| 165 | attagttgga gtaataataa cata | DNA |
| | | nucleotide |
| | | sequence |
| 166 | ISWSNNNI | AA amino acid |
| | | sequence |
| 167 | gcaaaatctg gaatctttga ctcc | DNA |
| | | nucleotide |
| | | sequence |
| 168 | AKSGIFDS | AA amino acid |
| | | sequence |
| 169 | gacatccaga tgacccagtc tccatcctcc ctgtctgcat ctgtaggaga | DNA |
| | cagagtcacc atcacttgcc gggcaagtca gagcattagc agctatttaa | nucleotide |
| | attggtatca gcagaaacca gggaagctcc tgatctatgc tgcatccagt | sequence |
| | ttgcaaagtg gggtcccatc acggttcagt ggcggtggat ctgggacaga | |
| | tttcactctc accatcagca gtctgcgacc tgaagatttt gcaacttact | |
| | actgtcaaca gagttactgt acccctccga tcaccttogg ccaagggaca | |
| | cgactggaga ttaaa | |
| 170 | DIQMTQSPSS LSASVGDRVT ITCRASQSIS SYLNWYQQKP GKLLIYAASS | AA amino acid |
| | LQSGVPSRFS GGGSGTDFTL TISSLRPEDF ATYYCQQSYC TPPITFGQGT | sequence |
| | RLEIK | |
| 171 | cagagcatta gcagctat | DNA |
| | | nucleotide |
| | | sequence |
| 172 | QSISSY | AA amino acid |
| | | sequence |
| 173 | gctgcatcc | DNA |
| | | nucleotide |
| | | sequence |
| 174 | AAS | AA amino acid |
| | | sequence |
| 175 | caacagagtt actgtacccc tccgatcacc | DNA |
| | | nucleotide |
| | | sequence |
| 176 | QQSYCTPPIT | AA amino acid |
| | | sequence |
| 177 | gaggtgcagc tggtggagtc cgggggaggc gtggtccagc ctgggaggtc | DNA |
| | cctgagactc tcctgtgcag cctctggatt caccttcagt agttatggca | nucleotide |
| | tgcactgggt ccgccaggct ccaggcaagg gactggagtg ggtgacactt | sequence |
| | atatcatatg agggaaggaa taaatactat gcagactccg tgaagggccg | |
| | attcaccatt tccagagaca attccaagaa cacgctgtat ctgcaaatga | |
| | acagcctgag agctgaggac acggctgtat attactgtgc gaaagatagg | |
| | accctttacg gtatggacgt ctggggccaa ggaaccacgg tcaccgtctc | |
| | ctca | |
| 178 | EVQLVESGGG VVQPGRSLRL SCAASGFTFS SYGMHWVRQA PGKGLEWVTL | AA amino acid |
| | ISYEGRNKYY ADSVKGRFTI SRDNSKNTLY LQMNSLRAED TAVYYCAKDR | sequence |
| | TLYGMDVWGQ GTTVTVSS | |
| 179 | ggattcacct tcagtagtta tggc | DNA |
| | | nucleotide |
| | | sequence |
| 180 | GFTFSSYG | AA amino acid |
| | | sequence |
| 181 | atatcatatg agggaaggaa taaa | DNA |
| | | nucleotide |
| | | sequence |
| 182 | ISYEGRNK | AA amino acid |
| | | sequence |
| 183 | gcgaaagata ggacccttta cggtatggac gtc | DNA |
| | | nucleotide |
| | | sequence |
| 184 | AKDRTLYGMD V | AA amino acid |
| | | sequence |
| 185 | caggtcacct tgagggagtc tggtcctgcg ctggtgaaaa ccacacagac | DNA |
| | cctcacactg acctgcacct tctctgggtt ctcactcagc actaatagaa | nucleotide |
| | tgtgtgtgac ctggatccgt cagcccccag ggaaggccct ggagtggctt | sequence |
| | gcgcgcattg attgggatgg tgttaaatac tacaacacat ctctgaagac | |
| | caggctcacc atctccaagg acacctccaa aaaccaggtg gtccttacaa | |
| | tgaccaacat ggaccctgtg gacacagcca ctttttactg tgcacggtcg | |
| | acttcgttga ctttttacta ctttgactac tggggccagg gaaccctggt | |
| | caccgtctcc tca | |
| 186 | QVTLRESGPA LVKTTQTLTL TCTFSGFSLS TNRMCVTWIR QPPGKALEWL | AA amino acid |
| | ARIDWDGVKY YNTSLKTRLT ISKDTSKNQV VLTMTNMDPV DTATFYCARS | sequence |
| | TSLTFYYFDY WGQGTLVTVS S | |
| 187 | gggttctcac tcagcactaa tagaatgtgt | DNA |
| | | nucleotide |
| | | sequence |
| 188 | GFSLSTNRMC | AA amino acid |
| | | sequence |
| 189 | attgattggg atggtgttaa a | DNA |
| | | nucleotide |
| | | sequence |
| 190 | IDWDGVK | AA amino acid |
| | | sequence |
| 191 | gcacggtcga cttcgttgac tttttactac tttgactac | DNA |
| | | nucleotide |
| | | sequence |
| 192 | ARSTSLTFYY FDY | AA amino acid |
| | | sequence |
| 193 | gacatccaga tgacccagtc tccatcctcc ctgtctgcat ctgtaggaga | DNA |
| | cagagtcacc atcacttgcc gggcaagtca gagcattagc agctatttaa | nucleotide |
| | attggtatca gcagaaacca gggaaagccc ctaagctcct gatctatgct | sequence |
| | gcatccagtt tgcaaagtgg ggtcccatca aggttcagtg gcagtggatc | |
| | tgggacagat ttcactctca ccatcagcag tctgcaacct gaagattttg | |
| | caacttacta ctgtcaacag agttacagta cccctccgat caccttcggc | |
| | caagggacac gactggagat taaa | |
| 194 | DIQMTQSPSS LSASVGDRVT ITCRASQSIS SYLNWYQQKP GKAPKLLIYA | AA amino acid |
| | ASSLQSGVPS RFSGSGSGTD FTLTISSLQP EDFATYYCQQ SYSTPPITFG | sequence |
| | QGTRLEIK | |
| 195 | cagagcatta gcagctat | DNA |
| | | nucleotide |
| | | sequence |
| 196 | QSISSY | AA amino acid |
| | | sequence |
| 197 | gctgcatcc | DNA |
| | | nucleotide |
| | | sequence |
| 198 | AAS | AA amino acid |
| | | sequence |
| 199 | caacagagtt acagtacccc tccgatcacc | DNA |
| | | nucleotide |
| | | sequence |
| 200 | QQSYSTPPIT | AA amino acid |
| | | sequence |
| 201 | gaggtgcagc tggtggagtc tggaggaggc ttggtccagc cgggggggtc | DNA |
| | cctgagactc tcctgtgcag cctctgagtt caccgtoggt accaaccaca | nucleotide |
| | tgaactgggt ccgccaggct ccagggaagg gactggagtg ggtctcagtt | sequence |
| | atttatagcg gtggtaacac attctacgca gactccgtga agggccgatt | |
| | caccatctcc agacacactt ccaagaacac gotgtatott caaatgaaca | |
| | gcctgacagc agaggacacg gccgtatatt actgtgcgcg aggattgggg | |
| | ggtatggacg tctggggcca agggaccacg gtcaccgtct cctca | |
| 202 | EVQLVESGGG LVQPGGSLRL SCAASEFTVG TNHMNWVRQA PGKGLEWVSV | AA amino acid |
| | IYSGGNTFYA DSVKGRFTIS RHTSKNTLYL QMNSLTAEDT AVYYCARGLG | sequence |
| | GMDVWGQGTT VTVSS | |
| 203 | gagttcaccg tcggtaccaa ccac | DNA |
| | | nucleotide |
| | | sequence |
| 204 | EFTVGTNH | AA amino acid |
| | | sequence |
| 205 | atttatagcg gtggtaacac a | DNA |
| | | nucleotide |
| | | sequence |
| 206 | IYSGGNT | AA amino acid |
| | | sequence |
| 207 | gcgcgaggat tggggggtat ggacgtc | DNA |
| | | nucleotide |
| | | sequence |
| 208 | ARGLGGMDV | AA amino acid |
| | | sequence |
| 209 | gacatccaga tgacccagtc tocatcctcc ctgtctgcat ctgtaggaga | DNA |
| | cagagtcacc atcacttgcc gggcgagtca ggtcattagc aattatttag | nucleotide |
| | cctggtatca gcagaaacca gggaaagttc ctaggctcct gatctatgct | sequence |
| | gcatccactt tgcaatcagg ggtcccatct cgtttcagtg gcagtggatc | |
| | tgggacagat ttcactctca ccatcagcag cctgcagcct gaagatgttg | |
| | caacttatta ctgtcaaaag tataacagtg cccctcggac gttoggccaa | |
| | gggaccaagg tggaaatcaa a | |
| 210 | DIQMTQSPSS LSASVGDRVT ITCRASQVIS NYLAWYQQKP GKVPRLLIYA | AA amino acid |
| | ASTLQSGVPS RFSGSGSGTD FTLTISSLQP EDVATYYCQK YNSAPRTFGQ | sequence |
| | GTKVEIK | |
| 211 | caggtcatta gcaattat | DNA |
| | | nucleotide |
| | | sequence |
| 212 | QVISNY | AA amino acid |
| | | sequence |
| 213 | gctgcatcc | DNA |
| | | nucleotide |
| | | sequence |
| 214 | AAS | AA amino acid |
| | | sequence |
| 215 | caaaagtata acagtgcccc toggacg | DNA |
| | | nucleotide |
| | | sequence |
| 216 | QKYNSAPRT | AA amino acid |
| | | sequence |
| 217 | gaggtgcagc tggtggagtc tgggggaggc ttggtccagc ggggggagtc | DNA |
| | cctgagactt tactgtgcag cctctggatt cacctttagt aaatattgga | nucleotide |
| | tgaactgggt ccgccaggct ccagggaagg ggctggagtg ggtggccaac | sequence |
| | ataaagggag atggaagtga gaaatactat gtggactctg tgaagggccg | |
| | gttcaccatc tccagagaca acgccaagaa ctcactatat ctacaaatga | |
| | acagcctgag agccgaggac acggctgttt attactgtgc gagagattat | |
| | tggggatcag gctactactt tgacttctgg ggccagggaa ccctggtcac | |
| | cgtctcctca | |
| 218 | EVQLVESGGG LVQRGESLRL YCAASGFTFS KYWMNWVRQA PGKGLEWVAN | AA amino acid |
| | IKGDGSEKYY VDSVKGRFTI SRDNAKNSLY LQMNSLRAED TAVYYCARDY | sequence |
| | WGSGYYFDFW GQGTLVTVSS | |
| 219 | ggattcacct ttagtaaata ttgg | DNA |
| | | nucleotide |
| | | sequence |
| 220 | GFTFSKYW | AA amino acid |
| | | sequence |
| 221 | ataaagggag atggaagtga gaaa | DNA |
| | | nucleotide |
| | | sequence |
| 222 | IKGDGSEK | AA amino acid |
| | | sequence |
| 223 | gcgagagatt attggggatc aggctactac tttgactto | DNA |
| | | nucleotide |
| | | sequence |
| 224 | ARDYWGSGYY FDF | AA amino acid |
| | | sequence |
| 225 | gacatccaga tgacccagtc tocatcctcc ctgtctgcat ctgtaggaga | DNA |
| | cagagtcacc atcacttgcc gggcaagtca gaacattaac aactatttaa | nucleotide |
| | attggtatca gcagaaacca gggaaagccc ctaaactcct gatctatgct | sequence |
| | gcatccagtt tccaaaatgc ggtcccatca aggttcagtg gcagtggatc | |
| | tgggacagat ttcactctca ccatcagcag tctgcaacct gaagattttg | |
| | caacttacta ctgtcaacag agttacaata ccccgctcac tttcggcggg | |
| | gggaccaagg tggagatcaa a | |
| 226 | DIQMTQSPSS LSASVGDRVT ITCRASQNIN NYLNWYQQKP GKAPKLLIYA | AA amino acid |
| | ASSFQNAVPS RFSGSGSGTD FTLTISSLQP EDFATYYCQQ SYNTPLTFGG | sequence |
| | GTKVEIK | |
| 227 | cagaacatta acaactat | DNA |
| | | nucleotide |
| | | sequence |
| 228 | QNINNY | AA amino acid |
| | | sequence |
| 229 | gctgcatcc | DNA |
| | | nucleotide |
| | | sequence |
| 230 | AAS | AA amino acid |
| | | sequence |
| 231 | caacagagtt acaatacccc gctcact | DNA |
| | | nucleotide |
| | | sequence |
| 232 | QQSYNTPLT | AA amino acid |
| | | sequence |
| 233 | gaggtgcagc tggtggagtc tgggggaggc ttggtccagt ctggggggtc | DNA |
| | cctgagactc tcctgtgcag cctctggatt cacctttagt agctattgga | nucleotide |
| | tgagctgggt ccgccaggct ccagggaagg ggctggagtg ggtggccaac | sequence |
| | ataaagcaag atggaagtga gaaatactat gtggactctg tgaagggccg | |
| | attcaccatc tocagagaca acgccaagaa ctcactgtat ctgcaaatga | |
| | acagcctgag agccgatgac acggctgtgt attactgtgc gagagatgat | |
| | attgtagtag taccagctcc tatgggatat tactactact acttcggtat | |
| | ggacgtctgg ggccaaggga ccacggtcac cgtctoctca | |
| 234 | EVQLVESGGG LVQSGGSLRL SCAASGFTFS SYWMSWVRQA PGKGLEWVAN | AA amino acid |
| | IKQDGSEKYY VDSVKGRFTI SRDNAKNSLY LQMNSLRADD TAVYYCARDD | sequence |
| | IVVVPAPMGY YYYYFGMDVW GQGTTVTVSS | |
| 235 | ggattcacct ttagtagcta ttgg | DNA |
| | | nucleotide |
| | | sequence |
| 236 | GFTFSSYW | AA amino acid |
| | | sequence |
| 237 | ataaagcaag atggaagtga gaaa | DNA |
| | | nucleotide |
| | | sequence |
| 238 | IKQDGSEK | AA amino acid |
| | | sequence |
| 239 | gcgagagatg atattgtagt agtaccagct cctatgggat attactacta | DNA |
| | ctacttoggt atggacgtc | nucleotide |
| | | sequence |
| 240 | ARDDIVVVPA PMGYYYYYFG MDV | AA amino acid |
| | | sequence |
| 241 | gacatccaga tgacccagtc tccatcctcc ctgtctgcat ctgtaggaga | DNA |
| | cagagtcacc atcacttgcc gggcaagtca gggcattaga aatgatttag | nucleotide |
| | gctggtatca gcagaaacca gggaaagccc ctaagcgcct gatctatgct | sequence |
| | gcatccagtt tgcaaagtgg ggtcccatca aggttcagcg gcagtggatc | |
| | tgggacagaa ttcactctca caatcagcag cctgcagcct gaagattttg | |
| | caacttatta ctgtctacag cataatagtt acccgtacac ttttggccag | |
| | gggaccaagc tggagatcaa a | |
| 242 | DIQMTQSPSS LSASVGDRVT ITCRASQGIR NDLGWYQQKP GKAPKRLIYA | AA amino acid |
| | ASSLQSGVPS RFSGSGSGTE FTLTISSLQP EDFATYYCLQ HNSYPYTFGQ | sequence |
| | GTKLEIK | |
| 243 | cagggcatta gaaatgat | DNA |
| | | nucleotide |
| | | sequence |
| 244 | QGIRND | AA amino acid |
| | | sequence |
| 245 | gctgcatcc | DNA |
| | | nucleotide |
| | | sequence |
| 246 | AAS | AA amino acid |
| | | sequence |
| 247 | ctacagcata atagttaccc gtacact | DNA |
| | | nucleotide |
| | | sequence |
| 248 | LQHNSYPYT | AA amino acid |
| | | sequence |
| 249 | gaagtgcagc tggtggagtc tgggggaggc ttggttcagc ctggcaggtc | DNA |
| | cctgagactc tcctgtgcag cctctggatt cacctttgat gattttgcca | nucleotide |
| | tgcactgggt ccgacaagct ccagggaagg gcctggagtg ggtctcaggt | sequence |
| | attagttgga ctggtggtaa catggactat gcgaactctg tgaagggccg | |
| | attcaccatc tccagagagg acgccaagaa ttccctgtat ctgcaaatga | |
| | acagtctgag agctgcggac acggccttgt attactgtgt aaaagatata | |
| | agggggatag tggctacggg gggggctttt gatatctggg gccgagggac | |
| | aatggtcacc gtctcttca | |
| 250 | EVQLVESGGG LVQPGRSLRL SCAASGFTFD DFAMHWVRQA PGKGLEWVSG | AA amino acid |
| | ISWTGGNMDY ANSVKGRFTI SREDAKNSLY LQMNSLRAAD TALYYCVKDI | sequence |
| | RGIVATGGAF DIWGRGTMVT VSS | |
| 251 | ggattcacct ttgatgattt tgcc | DNA |
| | | nucleotide |
| | | sequence |
| 252 | GFTFDDFA | AA amino acid |
| | | sequence |
| 253 | attagttgga ctggtggtaa catg | DNA |
| | | nucleotide |
| | | sequence |
| 254 | ISWTGGNM | AA amino acid |
| | | sequence |
| 255 | gtaaaagata taagggggat agtggctacg gggggggctt ttgatatc | DNA |
| | | nucleotide |
| | | sequence |
| 256 | VKDIRGIVAT GGAFDI | AA amino acid |
| | | sequence |
| 257 | gacatccaga tgacccagtc tccatcctcc ctgtctgcat ctgtaggaga | DNA |
| | cagagtcacc atctcttgcc gggcaagtca gaccattagc acttatttaa | nucleotide |
| | attggtttca gcagaaacca gggaaagccc ctaagctcct gatctatgtt | sequence |
| | gtgtccagtt tgcaaagtgg ggtcccatca aggttcagtg gcagtggatc | |
| | tgggacagat ttcactctca ccatcagcag tctgcaacct gaagattttg | |
| | caacttatta ctgtcaacag agttacagta ccccattcac tttcggccct | |
| | gggaccaaag tggatatcaa a | |
| 258 | DIQMTQSPSS LSASVGDRVT ISCRASQTIS TYLNWFQQKP GKAPKLLIYV | AA amino acid |
| | VSSLQSGVPS RFSGSGSGTD FTLTISSLQP EDFATYYCQQ SYSTPFTFGP | sequence |
| | GTKVDIK | |
| 259 | cagaccatta gcacttat | DNA |
| | | nucleotide |
| | | sequence |
| 260 | QTISTY | AA amino acid |
| | | sequence |
| 261 | gttgtgtcc | DNA |
| | | nucleotide |
| | | sequence |
| 262 | VVS | AA amino acid |
| | | sequence |
| 263 | caacagagtt acagtacccc attcact | DNA |
| | | nucleotide |
| | | sequence |
| 264 | QQSYSTPFT | AA amino acid |
| | | sequence |
| 265 | gaggtgcagc tggtggagtc tggaggaggc ttggtccagc cgggggggtc | DNA |
| | cctgagactc tcctgtgcag cctctggatt caccgtoggt accaactaca | nucleotide |
| | tgaactgggt ccgccaggct ccagggaagg gactggagtg gatctcagtt | sequence |
| | atttatagcg gtggtagcac attctacgca gactccgtga agggccgatt | |
| | caccatctcc agacagactt cccagaacac gctgtatott caaatgaaca | |
| | gcctgagacc tgaggacacg gccgtatatt actgtgcgag aggtatacgt | |
| | ggttttgata tctggggcca agggacaatg gtcaccgtct cttca | |
| 266 | EVQLVESGGG LVQPGGSLRL SCAASGFTVG TNYMNWVRQA PGKGLEWISV | AA amino acid |
| | IYSGGSTFYA DSVKGRFTIS RQTSQNTLYL QMNSLRPEDT AVYYCARGIR | sequence |
| | GFDIWGQGTM VTVSS | |
| 267 | ggattcaccg tcggtaccaa ctac | DNA |
| | | nucleotide |
| | | sequence |
| 268 | GFTVGTNY | AA amino acid |
| | | sequence |
| 269 | atttatagcg gtggtagcac a | DNA |
| | | nucleotide |
| | | sequence |
| 270 | IYSGGST | AA amino acid |
| | | sequence |
| 271 | gcgagaggta tacgtggttt tgatatc | DNA |
| | | nucleotide |
| | | sequence |
| 272 | ARGIRGFDI | AA amino acid |
| | | sequence |
| 273 | gacatccaga tgacccagtc tocatcctcc ctgtctgcat ctgtaggaga | DNA |
| | cagagtcacc atcacttgcc gggcaagtca gagcattagc agctatttaa | nucleotide |
| | attggtatca gcagaaacca gggaaagccc ctaagctcct gatctatgct | sequence |
| | gcatccagtt tgcaaagtgg ggtcccgtca aggttcagtg gcagtggatc | |
| | tgggacagat ttcactctca ccatcagcag tctgcaacct gaagattttg | |
| | caacttacta ctgtcaacag agttacagta cccctccgat caccttcggc | |
| | caagggacac gactggagat taaa | |
| 274 | DIQMTQSPSS LSASVGDRVT ITCRASQSIS SYLNWYQQKP GKAPKLLIYA | AA amino acid |
| | ASSLQSGVPS RFSGSGSGTD FTLTISSLQP EDFATYYCQQ SYSTPPITFG | sequence |
| | QGTRLEIK | |
| 275 | cagagcatta gcagctat | DNA |
| | | nucleotide |
| | | sequence |
| 276 | QSISSY | AA amino acid |
| | | sequence |
| 277 | gctgcatcc | DNA |
| | | nucleotide |
| | | sequence |
| 278 | AAS | AA amino acid |
| | | sequence |
| 279 | caacagagtt acagtacccc tccgatcacc | DNA |
| | | nucleotide |
| | | sequence |
| 280 | QQSYSTPPIT | AA amino acid |
| | | sequence |
| 281 | gaggtgcagc tggtggagtc tggaggaggc ttggtccagc cgggggggtc | DNA |
| | cctgagactc tcctgtgcag cctctgggtt taccatcagt accaactaca | nucleotide |
| | tgaactgggt ccgccaggct ccagggaagg ggctggagtg ggtcgcagtt | sequence |
| | atttatagca gtggttccac atactatatc gactccgtga agggccgatt | |
| | caccatctcc agactcactt ccaagaacac ggtgtatott caaatgagca | |
| | gcctgaattc tgaagacacg gccgtgtatt actgtgcgag ggggatcagg | |
| | ggttttgata tttggggcca agggacaatg gtcaccgtct cttca | |
| 282 | EVQLVESGGG LVQPGGSLRL SCAASGFTIS TNYMNWVRQA PGKGLEWVAV | AA amino acid |
| | IYSSGSTYYI DSVKGRFTIS RLTSKNTVYL QMSSLNSEDT AVYYCARGIR | sequence |
| | GFDIWGQGTM VTVSS | |
| 283 | gggtttacca tcagtaccaa ctac | DNA |
| | | nucleotide |
| | | sequence |
| 284 | GFTISTNY | AA amino acid |
| | | sequence |
| 285 | atttatagca gtggttccac a | DNA |
| | | nucleotide |
| | | sequence |
| 286 | IYSSGST | AA amino acid |
| | | sequence |
| 287 | gcgaggggga tcaggggttt tgatatt | DNA |
| | | nucleotide |
| | | sequence |
| 288 | ARGIRGFDI | AA amino acid |
| | | sequence |
| 289 | gaagtgcagc tggtggagtc ggggggaggc ttggtacagc ctggcaggtc | DNA |
| | cctgagactc tectgtgcag cctctggatt caccattgat gatagtgcca | nucleotide |
| | tgcactgggt ccggcaaact ccagggaagg gcctggagtg ggtctcaggt | sequence |
| | attagttgga aaagtggtag cataggttat gcggactctg tgaggggccg | |
| | attcaccato tccagagaca acgccaagaa ttccctctat ctgcaaatga | |
| | acagtctgag agttgaggac acggccttgt attactgtgt aaaagatata | |
| | aggggcaact ggaactacgg gggaaactgg ttcgacccct ggggccaggg | |
| | aaccctggtc actgtctcct ca | |
| 290 | EVQLVESGGG LVQPGRSLRL SCAASGFTID DSAMHWVRQT PGKGLEWVSG | AA amino acid |
| | ISWKSGSIGY ADSVRGRFTI SRDNAKNSLY LQMNSLRVED TALYYCVKDI | sequence |
| | RGNWNYGGNW FDPWGQGTLV TVSS | |
| 291 | ggattcacca ttgatgatag tgcc | DNA |
| | | nucleotide |
| | | sequence |
| 292 | GFTIDDSA | AA amino acid |
| | | sequence |
| 293 | attagttgga aaagtggtag cata | DNA |
| | | nucleotide |
| | | sequence |
| 294 | ISWKSGSI | AA amino acid |
| | | sequence |
| 295 | gtaaaagata taaggggcaa ctggaactac gggggaaact ggttegaccc | DNA |
| | c | nucleotide |
| | | sequence |
| 296 | VKDIRGNWNY GGNWFDP | AA amino acid |
| | | sequence |
| 297 | gaggtgcagc tggtggagtc tggaggaggc ttggtccagc ctggggggtc | DNA |
| | cctgagactc tcatgtgaag cctctgggtt caccgtcggt gtcaaccaca | nucleotide |
| | tgaactgggt ccgccaggct ccagggaagg gtctggagtg ggtctcagtt | sequence |
| | attttcagta gtggtaggac attctacgga gactacgtga aggggcgatt | |
| | aaccatcttc agacaaacct cccagaacac ggtgtatott caaatgaata | |
| | gcctgagaag tgaggacacg gccatatatt actgtgcgag agggattggc | |
| | ggtttggaca tctggggccg agggacaatg gtcaccgtct cttca | |
| 298 | EVQLVESGGG LVQPGGSLRL SCEASGFTVGV NHMNWVRQA PGKGLEWVSV | AA amino acid |
| | IFSSGRTFYG DYVKGRLTIF RQTSQNTVYL QMNSLRSEDT AIYYCARGIG | sequence |
| | GLDIWGRGTM VTVSS | |
| 299 | gggttcaccg toggtgtcaa ccac | DNA |
| | | nucleotide |
| | | sequence |
| 300 | GFTVGVNH | AA amino acid |
| | | sequence |
| 301 | attttcagta gtggtaggac a | DNA |
| | | nucleotide |
| | | sequence |
| 302 | IFSSGRT | AA amino acid |
| | | sequence |
| 303 | gcgagaggga ttggcggttt ggacatc | DNA |
| | | nucleotide |
| | | sequence |
| 304 | ARGIGGLDI | AA amino acid |
| | | sequence |
| 305 | gaagtgcagc tggtggagtc tgggggaggc ttggttcagc ctggcaggtc | DNA |
| | cctaagactc tcctgtgcag cctctggatt cacctttgat gattatgcct | nucleotide |
| | tgcactgggt ccggcaagct ccagggaagg gcctggagtg ggtctcaggt | sequence |
| | attagttgga ctggtggtac tatagactat gcggactctg tgaagggccg | |
| | attcaccatc tccagagaca acgccaagaa ctccctgtat ctgcaaatga | |
| | gcagtctgag aactgaggac acggccatat attactgtac aagagatatc | |
| | cgggggaact ggaagtacgg aggctggttc gacccctggg gccagggaac | |
| | cctggtcacc gtctcctca | |
| 306 | EVQLVESGGG LVQPGRSLRL SCAASGFTFD DYALHWVRQA PGKGLEWVSG | AA amino acid |
| | ISWTGGTIDY ADSVKGRFTI SRDNAKNSLY LQMSSLRTED TAIYYCTRDI | sequence |
| | RGNWKYGGWF DPWGQGTLVT VSS | |
| 307 | ggattcacct ttgatgatta tgcc | DNA |
| | | nucleotide |
| | | sequence |
| 308 | GFTFDDYA | AA amino acid |
| | | sequence |
| 309 | attagttgga ctggtggtac tata | DNA |
| | | nucleotide |
| | | sequence |
| 310 | ISWTGGTI | AA amino acid |
| | | sequence |
| 311 | acaagagata tccgggggaa ctggaagtac ggaggctggt tcgacccc | DNA |
| | | nucleotide |
| | | sequence |
| 312 | TRDIRGNWKY GGWFDP | AA amino acid |
| | | sequence |
| 313 | caggtgcagc tggtgcagtc tgggactgag gtgaagaagc ctggggcctc | DNA |
| | agtgaaggtc tcctgcaagg cttctggata caccttcacc gcctactata | nucleotide |
| | tgcactgggt gcgacaggcc cctggtcaag gacttgactg gatgggatgg | sequence |
| | atcagcccta acagtggttt cacaaactat gcacagaagt ttcagggcag | |
| | ggtcaccatg accagggaca cgtccatcaa cacattttat atggagctga | |
| | gtggactgag atctgacgac acggccgtat attactgtgc gcgagagggt | |
| | tctactcacc acaattcttt cgacccctgg ggccagggaa ccctggtcac | |
| | cgtctcctca | |
| 314 | QVQLVQSGTE VKKPGASVKV SCKASGYTFT AYYMHWVRQA PGQGLDWMGW | AA amino acid |
| | ISPNSGFTNY AQKFQGRVTM TRDTSINTFY MELSGLRSDD TAVYYCAREG | sequence |
| | STHHNSFDPW GQGTLVTVSS | |
| 315 | ggatacacct tcaccgccta ctat | DNA |
| | | nucleotide |
| | | sequence |
| 316 | GYTFTAYY | AA amino acid |
| | | sequence |
| 317 | atcagcccta acagtggttt caca | DNA |
| | | nucleotide |
| | | sequence |
| 318 | ISPNSGFT | AA amino acid |
| | | sequence |
| 319 | gcgcgagagg gttctactca ccacaattct ttcgacccc | DNA |
| | | nucleotide |
| | | sequence |
| 320 | AREGSTHHNS FDP | AA amino acid |
| | | sequence |
| 321 | gaggtgcagc tggtggagtc tggaggaggc ttggtccaac cgggggggtc | DNA |
| | cctgaggctc tcctgtgcag cctctgggtt caccgtcggt actaacttca | nucleotide |
| | tgaattgggt ccgccaggct ccagggaagg ggctggagtg ggtctcagcg | sequence |
| | atttatagcg gtggtaccgc taactacgca gactccgtga agggccgatt | |
| | caccatttcc agagacactt ccaggaacac gctgtatott caaatgaaca | |
| | gcctgagaac tgaggacacg gccgtttatt attgtgcgag aggggggggt | |
| | atggacgtct ggggccaagg gaccacggtc accgtctect ca | |
| 322 | EVQLVESGGG LVQPGGSLRL SCAASGFTVG TNFMNWVRQA PGKGLEWVSA | AA amino acid |
| | IYSGGTANYA DSVKGRFTIS RDTSRNTLYL QMNSLRTEDT AVYYCARGGG | sequence |
| | MDVWGQGTTV TVSS | |
| 323 | gggttcaccg toggtactaa cttc | DNA |
| | | nucleotide |
| | | sequence |
| 324 | GFTVGTNF | AA amino acid |
| | | sequence |
| 325 | atttatagcg gtggtaccgc t | DNA |
| | | nucleotide |
| | | sequence |
| 326 | IYSGGTA | AA amino acid |
| | | sequence |
| 327 | gcgagagggg ggggtatgga cgtc | DNA |
| | | nucleotide |
| | | sequence |
| 328 | ARGGGMDV | AA amino acid |
| | | sequence |
| 329 | caggtccagc tggtgcagtc tggggctgag gtgaagaagc ctgggtcctc | DNA |
| | ggtgaaggtc tcctgcaagg cttctggagg caccttcaac acctatgttc | nucleotide |
| | tcagctgggt gcgacaggcc cctggacaag ggcttgagtg gatgggagag | sequence |
| | atcatcccta tcttaggtgc agcaaactac gcacagaact tccagggcag | |
| | agtcactttt accacggacg aatccacgaa tacagcctac atggacctga | |
| | gcagcctaag atctgaggac acggccgtgt attactgtgc gagagatcgg | |
| | acctccgggg ggttcgaccc ctggggccag ggaaccctgg tcactgtctc | |
| | ctca | |
| 330 | QVQLVQSGAE VKKPGSSVKV SCKASGGTFN TYVLSWVRQA PGQGLEWMGE | AA amino acid |
| | IIPILGAANY AQNFQGRVTF TTDESTNTAY MDLSSLRSED TAVYYCARDR | sequence |
| | TSGGFDPWGQ GTLVTVSS | |
| 331 | ggaggcacct tcaacaccta tgtt | DNA |
| | | nucleotide |
| | | sequence |
| 332 | GGTFNTYV | AA amino acid |
| | | sequence |
| 333 | atcatcccta tottaggtgc agca | DNA |
| | | nucleotide |
| | | sequence |
| 334 | IIPILGAA | AA amino acid |
| | | sequence |
| 335 | gcgagagatc ggacctccgg ggggttcgac ccc | DNA |
| | | nucleotide |
| | | sequence |
| 336 | ARDRTSGGFD P | AA amino acid |
| | | sequence |
| 337 | caggttcagc tggtgcagtc tggagctgag gtggagaagc ctggggcctc | DNA |
| | agtgaaggtc tcctgcaagg cttctggtta catctttacc cactatggta | nucleotide |
| | tcagctgggt gcgacaggcc cctggacaag gacttgagtg ggtgggctgg | sequence |
| | atcagccctt acaatggtta cacagactat gcacagaaac tccagggcag | |
| | agtcaccttg accacagaca catccacgac cacagcctac atggagctga | |
| | ggaacctgag atctgacgac acggccatgt attactgttc gagagggagg | |
| | ggcccttact ggtccttcga tctctggggc cgtggcaccc tggtcaccgt | |
| | ctcctca | |
| 338 | QVQLVQSGAE VEKPGASVKV SCKASGYIFT HYGISWVRQA PGQGLEWVGW | AA amino acid |
| | ISPYNGYTDY AQKLQGRVTL TTDTSTTTAY MELRNLRSDD TAMYYCSRGR | sequence |
| | GPYWSFDLWG RGTLVTVSS | |
| 339 | ggttacatct ttacccacta tggt | DNA |
| | | nucleotide |
| | | sequence |
| 340 | GYIFTHYG | AA amino acid |
| | | sequence |
| 341 | atcagccctt acaatggtta caca | DNA |
| | | nucleotide |
| | | sequence |
| 342 | ISPYNGYT | AA amino acid |
| | | sequence |
| 343 | tcgagaggga ggggccctta ctggtccttc gatctc | DNA |
| | | nucleotide |
| | | sequence |
| 344 | SRGRGPYWSF DL | AA amino acid |
| | | sequence |

What is claimed is:

1. A radiolabeled antibody conjugate comprising an antibody or antigen binding fragment thereof that binds monomeric human program death ligand 1 (PD-L1), a chelating moiety, and a positron emitter.

2. The conjugate of claim 1, wherein said conjugate comprises an antibody or antigen-binding fragment thereof that binds PD-L1, wherein said antibody or antigen-binding fragment thereof is covalently bonded to one or more moieties of formula (A):
-L-M_{Z} (A)
wherein L is a chelating moiety; M is a positron emitter; and z, independently at each occurrence, is 0 or 1; and wherein at least one of z is 1.

3. The conjugate of claim 1, wherein the chelating moiety comprises desferrioxamine.

4. The conjugate of claim 1, wherein the positron emitter is ⁸⁹Zr.

5. The conjugate of claim 2, wherein -L-M is

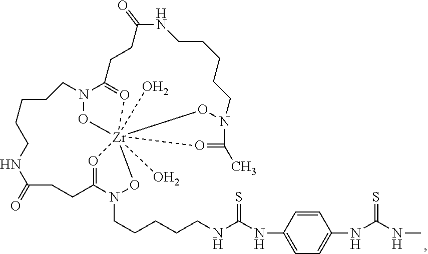

6. The compound of claim 1, wherein antibody or antigen-binding fragment thereof is covalently bonded to one, two, or three moieties of Formula (A).

7. The conjugate of claim 1, wherein the antibody has one or more properties selected from the group consisting of:
(a) binds monomeric PD-L1 with a binding dissociation equilibrium constant (K_{D}) of less than about 310 pM as measured in a surface plasmon resonance assay at 37° C.;
(b) binds monomeric human PD-L1 with a K_{D} less than about 180 pM in a surface plasmon resonance assay at 25° C.;
(c) binds dimeric human PD-L1 with a K_{D} of less than about 15 pM as measured in a surface plasmon resonance assay at 37° C.; and
(d) binds dimeric human PD-L1 with a K_{D} less than about 8 pM in a surface plasmon resonance assay at 25°.

8. The conjugate of claim 1, wherein the antibody comprises three heavy chain complementarity determining regions (HCDRs) in a heavy chain variable region (HCVR), wherein the HCVR has an amino acid sequence selected from the group consisting of SEQ ID NOs: 2, 34, 50, 82, 98, 146, 162, 178, 186, 234, 250, 266, 290, 306, 314, and 330; and three light chain complementarity determining regions (LCDRs) in a light chain variable region (LCVR), wherein the LCVR has an amino acid sequence selected from the group consisting of SEQ ID NOs: 10, 42, 58, 90, 106, 154, 170, 194, 242, 258, and 274.

9. The conjugate of claim 1, wherein the antibody comprises three CDRs in a HCVR of SEQ ID NO: 82; and three CDRs in a LCVR of SEQ ID NO: 90.

10. A method of imaging a tissue that expresses PD-L1 comprising administering a radiolabeled antibody conjugate of claim 1 to the tissue; and visualizing PD-L1 expression by positron emission tomography (PET) imaging.

11. A method for treating a tumor comprising:
(a) selecting a subject with a solid tumor;
(b) determining that the solid tumor is PD-L1-positive; and
(c) administering one or more doses of an inhibitor of the PD-1/PD-L1 signaling axis to the subject in need thereof.

12. The method of claim 11, wherein step (b) comprises:
(i) administering a radiolabeled antibody conjugate of claim 1 to the subject in need thereof; and
(ii) imaging localization of the radiolabeled antibody conjugate in the tumor by positron emission tomography (PET) imaging, wherein presence of the radiolabeled antibody conjugate in the tumor indicates that the tumor is PD-L1-positive.

13. The method of claim 12, wherein the subject is administered 0.1-10 mg/kg of the radiolabeled antibody conjugate.

14. The method of claim 12, wherein the radiolabeled antibody conjugate is administered sub-cutaneously or intravenously to the subject.

15. The method of claim 12, wherein PET imaging is done 2-7 days after administering the radiolabeled antibody conjugate.

16. The method of claim 12, wherein step (b) is carried out before treating the subject with an inhibitor of the PD-1/PD-L1 signaling axis.

17. The method of claim 12 further comprising:
(a) administering the radiolabeled antibody conjugate after treating the subject with at least one dose of an inhibitor of the PD-1/PD-L1 signaling axis; and
(b) imaging localization of the radiolabeled antibody conjugate in the tumor by PET imaging, wherein a decrease from the baseline in the area of localization of the radiolabeled antibody conjugate in the tumor indicates tumor regression.

18. The method of claim 17, wherein the subject is administered the radiolabeled antibody conjugate 1-20 weeks after administration of the inhibitor of the PD-1/PD-L1 signaling axis.

19. The method of claim 11, wherein the tumor is selected from the group consisting of blood cancer, brain cancer, renal cell cancer, ovarian cancer, bladder cancer, prostate cancer, breast cancer, hepatic cell carcinoma, bone cancer, colon cancer, non-small-cell lung cancer, squamous cell carcinoma of head and neck, colorectal cancer, mesothelioma, B cell lymphoma, and melanoma.

20. The method of claim 11, wherein the inhibitor of the PD-1/PD-L1 signaling axis is an anti-PD-1 antibody or antigen-binding fragment thereof.

21. The method of claim 20, wherein the anti-PD-1 antibody is selected from the group consisting of nivolumab, pembrolizumab and REGN2810.

22. The method of claim 11, wherein the inhibitor of the PD-1/PD-L1 signaling axis is an anti-PD-L1 antibody or antigen-binding fragment thereof.

23. The method of claim 22, wherein the anti-PD-L1 antibody is atezolizumab.

24. The method of claim 22, wherein the anti-PD-L1 antibody or antigen-binding fragment thereof comprises three heavy chain complementarity determining regions (HCDRs) in a heavy chain variable region (HCVR) of SEQ ID NO: 82; and three light chain complementarity determining regions (LCDRs) in a light chain variable region (LCVR) of SEQ ID NO: 90.

25. The method of claim 22, wherein the anti-PD-L1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 84; the HCDR2 comprises the amino acid sequence of SEQ ID NO: 86; the HCDR3 comprises the amino acid sequence of SEQ ID NO: 88; the LCDR1 comprises the amino acid sequence of SEQ ID NO: 92; the LCDR2 comprises the amino acid sequence of SEQ ID NO: 94; and the LCDR3 comprises the amino acid sequence of SEQ ID NO: 96.

26. A compound of Formula (III): 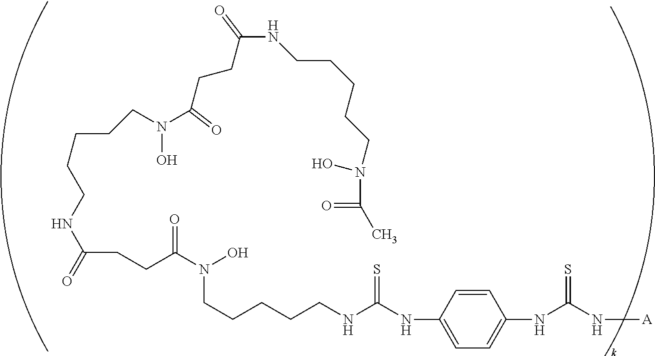
wherein A is an antibody or antigen binding fragment thereof that binds PD-L1 and k is an integer from 1-30.


SEQUENCE LISTING (344 sequences)

SEQ ID NO: 1 - DNA, 363 bp, synthetic construct
  misc_feature 1..363: note = synthetic
  source 1..363: mol_type = other DNA
gaggtgcagctggtggagtctgggggaggcttggtccagcctggggggtccctgagactctcctgtgcagcctctggattcacctttagtaggttttggatgagctgggtccgccaggctccagggaaggggctggagtgggtggccaacataaaccaagatggaactgagaaatactatgtggactctgtgaagggccgattcaccatctccagagacaacgccaagaactcactgtatctgcaaatgaacagcctgagagccggggacacggctgtgtattactgtgcgaatacgtattacgatttttggagtggtcactttgactactggggccagggaaccctggtcaccgtctcctca

SEQ ID NO: 2 - protein, 121 aa, synthetic construct
  REGION 1..121: note = synthetic
EVQLVESGGGLVQPGGSLRLSCAASGFTFSRFWMSWVRQAPGKGLEWVANINQDGTEKYYVDSVKGRFTISRDNAKNSLYLQMNSLRAGDTAVYYCANTYYDFWSGHFDYWGQGTLVTVSS

SEQ ID NO: 3 - DNA, 24 bp, synthetic construct
  misc_feature 1..24: note = synthetic
  source 1..24: mol_type = other DNA
ggattcacctttagtaggttttgg

SEQ ID NO: 4 - protein, 8 aa, synthetic construct
  REGION 1..8: note = synthetic
GFTFSRFW

SEQ ID NO: 5 - DNA, 24 bp, synthetic construct
  misc_feature 1..24: note = synthetic
  source 1..24: mol_type = other DNA
ataaaccaagatggaactgagaaa

SEQ ID NO: 6 - protein, 8 aa, synthetic construct
  REGION 1..8: note = synthetic
INQDGTEK

SEQ ID NO: 7 - DNA, 42 bp, synthetic construct
  misc_feature 1..42: note = synthetic
  source 1..42: mol_type = other DNA
gcgaatacgtattacgatttttggagtggtcactttgactac

SEQ ID NO: 8 - protein, 14 aa, synthetic construct
  REGION 1..14: note = synthetic
ANTYYDFWSGHFDY

SEQ ID NO: 9 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgtcgggccagtcagagtattagtaattggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcatagttattcgtacacttttggccaggggaccaagctggagatcaaa

SEQ ID NO: 10 - protein, 107 aa, synthetic construct
  REGION 1..107: note = synthetic
DIQMTQSPSTLSASVGDRVTITCRASQSISNWLAWYQQKPGKAPKLLIYKASSLESGVPSRFSGSGSGTEFTLTISSLQPDDFATYYCQQYHSYSYTFGQGTKLEIK

SEQ ID NO: 11 - DNA, 18 bp, synthetic construct
  misc_feature 1..18: note = synthetic
  source 1..18: mol_type = other DNA
cagagtattagtaattgg

SEQ ID NO: 12 - protein, 6 aa, synthetic construct
  REGION 1..6: note = synthetic
QSISNW

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagtatcatagttattcgtacact

SEQ ID NO: 16 - protein, 9 aa, synthetic construct
  REGION 1..9: note = synthetic
QQYHSYSYT

SEQ ID NO: 17 - DNA, 363 bp, synthetic construct
  misc_feature 1..363: note = synthetic
  source 1..363: mol_type = other DNA
caggagcacctggtggagtctgggggaggcgtggtccagcctgggaggtccctgagactctcctgtgaagcgtctggattcaccttcagtaactttggcatgcactgggtccgccaggctccaggcaaggggctggagtgggtggcagctttatggtctgatggaagtaataaatactatgcagactccgtgaagggtcgagtcaccatctccagagacaattccaagaacacactgtatctgcaaatgaacagcctgagagccgaggacacggctgtctattactgtgcgagagggagaggagcccccggtattccgatttttgggtactggggccagggaaccctggtcaccgtctcctca

SEQ ID NO: 18 - protein, 121 aa, synthetic construct
  REGION 1..121: note = synthetic
QEHLVESGGGVVQPGRSLRLSCEASGFTFSNFGMHWVRQAPGKGLEWVAALWSDGSNKYYADSVKGRVTISRDNSKNTLYLQMNSLRAEDTAVYYCARGRGAPGIPIFGYWGQGTLVTVSS

SEQ ID NO: 19 - DNA, 24 bp, synthetic construct
  misc_feature 1..24: note = synthetic
  source 1..24: mol_type = other DNA
ggattcaccttcagtaactttggc

SEQ ID NO: 20 - protein, 8 aa, synthetic construct
  REGION 1..8: note = synthetic
GFTFSNFG

SEQ ID NO: 21 - DNA, 24 bp, synthetic construct
  misc_feature 1..24: note = synthetic
  source 1..24: mol_type = other DNA
ttatggtctgatggaagtaataaa

SEQ ID NO: 22 - protein, 8 aa, synthetic construct
  REGION 1..8: note = synthetic
LWSDGSNK

SEQ ID NO: 23 - DNA, 42 bp, synthetic construct
  misc_feature 1..42: note = synthetic
  source 1..42: mol_type = other DNA
gcgagagggagaggagcccccggtattccgatttttgggtac

SEQ ID NO: 24 - protein, 14 aa, synthetic construct
  REGION 1..14: note = synthetic
ARGRGAPGIPIFGY

SEQ ID NO: 25 - DNA, 321 bp, synthetic construct
  misc_feature 1..321: note = synthetic
  source 1..321: mol_type = other DNA
gacatccagatgacccagtctccatcctccctgtctgcatctgttggagacagagtcaccatcacttgccgggcaagtcagggcattagaaatgatttaggctggtatcagcagaaaccagggaaagcccctaagcgcctgatctatactgcatccagtttgcaaagtggggtcccatcaagattcagcggcagtggatctgggacagaattcactctcacaatcagcagcctacagcctgaagattttgcaacttattactgtctacaacataatagttaccctctcacattcggcggagggaccaaggtggcgatcaaa

SEQ ID NO: 26 - protein, 107 aa, synthetic construct
  REGION 1..107: note = synthetic
DIQMTQSPSSLSASVGDRVTITCRASQGIRNDLGWYQQKPGKAPKRLIYTASSLQSGVPSRFSGSGSGTEFTLTISSLQPEDFATYYCLQHNSYPLTFGGGTKVAIK

SEQ ID NO: 27 - DNA, 18 bp, synthetic construct
  misc_feature 1..18: note = synthetic
  source 1..18: mol_type = other DNA
cagggcattagaaatgat

SEQ ID NO: 28 - protein, 6 aa, synthetic construct
  REGION 1..6: note = synthetic
QGIRND

SEQ ID NO: 29-30 - skipped (2 entries)

SEQ ID NO: 31 - DNA, 27 bp, synthetic construct
  misc_feature 1..27: note = synthetic
  source 1..27: mol_type = other DNA
ctacaacataatagttaccctctcaca

SEQ ID NO: 32 - protein, 9 aa, synthetic construct
  REGION 1..9: note = synthetic
LQHNSYPLT

SEQ ID NO: 33 - DNA, 390 bp, synthetic construct
  misc_feature 1..390: note = synthetic
  source 1..390: mol_type = other DNA
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaaggaaaactgatggtgggacaacagactacgctgcacccgtgaaaggcagattcaccatctcaagagatgattcaaaaaatacgctgcatctgcaaatgaacagcctgaaaaccgaggacacagccgtgtattactgtaccacagatgatattgtagttgtaccagctgttatgagggaatactacttcggtatggacgtctggggccaagggaccacggtcaccgtctcctca

SEQ ID NO: 34 - protein, 130 aa, synthetic construct
  REGION 1..130: note = synthetic
EVQLVESGGGLVKPGGSLRLSCAASGFTFSNAWMSWVRQAPGKGLEWVGRIKRKTDGGTTDYAAPVKGRFTISRDDSKNTLHLQMNSLKTEDTAVYYCTTDDIVVVPAVMREYYFGMDVWGQGTTVTVSS

SEQ ID NO: 35 - DNA, 24 bp, synthetic construct
  misc_feature 1..24: note = synthetic
  source 1..24: mol_type = other DNA
ggattcactttcagtaacgcctgg

SEQ ID NO: 36 - protein, 8 aa, synthetic construct
  REGION 1..8: note = synthetic
GFTFSNAW

SEQ ID NO: 37 - DNA, 30 bp, synthetic construct
  misc_feature 1..30: note = synthetic
  source 1..30: mol_type = other DNA
attaaaaggaaaactgatggtgggacaaca

SEQ ID NO: 38 - protein, 10 aa, synthetic construct
  REGION 1..10: note = synthetic
IKRKTDGGTT

SEQ ID NO: 39 - DNA, 63 bp, synthetic construct
  misc_feature 1..63: note = synthetic
  source 1..63: mol_type = other DNA
accacagatgatattgtagttgtaccagctgttatgagggaatactacttcggtatggacgtc

SEQ ID NO: 40 - protein, 21 aa, synthetic construct
  REGION 1..21: note = synthetic
TTDDIVVVPAVMREYYFGMDV

SEQ ID NO: 41 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccggacaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataataattacccgtacacttttggccaggggaccaagctggagatcaaa

SEQ ID NO: 42 - protein, 107 aa, synthetic construct
  REGION 1..107: note = synthetic
DIQMTQSPSSLSASVGDRVTITCRTSQGIRNDLGWYQQKPGKAPKRLIYAASSLQSGVPSRFSGSGSGTEFTLTISSLQPEDFATYYCLQHNNYPYTFGQGTKLEIK

SEQ ID NO: 43 - DNA, 18 bp, synthetic construct
  misc_feature 1..18: note = synthetic
  source 1..18: mol_type = other DNA
cagggcattagaaatgat

SEQ ID NO: 44 - protein, 6 aa, synthetic construct
  REGION 1..6: note = synthetic
QGIRND

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
ctacagcataataattacccgtacact

SEQ ID NO: 48 - protein, 9 aa, synthetic construct
  REGION 1..9: note = synthetic
LQHNNYPYT

SEQ ID NO: 49 - DNA, 363 bp, synthetic construct
  misc_feature 1..363: note = synthetic
  source 1..363: mol_type = other DNA
caggtgcaattggtgcagtctggggcggaggtgaagaagcctggggcctcagtgcaggtctcctgcaaggcttctggatactccttcaccggctactatatacactgggtgcgacaggcccctggacaaggacttgagtggatgggatggatcaaccctaacagtggcaccaaaaagtatgcacacaagtttcagggcagggtcaccatgaccagggacacgtccatcgacacagcctacatgattttgagcagtctgatatccgacgacacggccgtgtattactgtgcgagagatgaggactggaactttgggagctggttcgactcctggggccagggaaccctggtcaccgtctcctca

SEQ ID NO: 50 - protein, 121 aa, synthetic construct
  REGION 1..121: note = synthetic
QVQLVQSGAEVKKPGASVQVSCKASGYSFTGYYIHWVRQAPGQGLEWMGWINPNSGTKKYAHKFQGRVTMTRDTSIDTAYMILSSLISDDTAVYYCARDEDWNFGSWFDSWGQGTLVTVSS

SEQ ID NO: 51 - DNA, 24 bp, synthetic construct
  misc_feature 1..24: note = synthetic
  source 1..24: mol_type = other DNA
ggatactccttcaccggctactat

SEQ ID NO: 52 - protein, 8 aa, synthetic construct
  REGION 1..8: note = synthetic
GYSFTGYY

SEQ ID NO: 53 - DNA, 24 bp, synthetic construct
  misc_feature 1..24: note = synthetic
  source 1..24: mol_type = other DNA
atcaaccctaacagtggcaccaaa

SEQ ID NO: 54 - protein, 8 aa, synthetic construct
  REGION 1..8: note = synthetic
INPNSGTK

SEQ ID NO: 55 - DNA, 42 bp, synthetic construct
  misc_feature 1..42: note = synthetic
  source 1..42: mol_type = other DNA
gcgagagatgaggactggaactttgggagctggttcgactcc

SEQ ID NO: 56 - protein, 14 aa, synthetic construct
  REGION 1..14: note = synthetic
ARDEDWNFGSWFDS

SEQ ID NO: 57 - DNA, 336 bp, synthetic construct
  misc_feature 1..336: note = synthetic
  source 1..336: mol_type = other DNA
gatattgtgatgacccagactccactctcctcacctgtcacccttggacagccggcctccatctcctgcaggtctagtcaaaccctcgtacacggtgatggaaacacgtacttgagttggattcagcagaggccaggccagcctccgagactcctcatttataaggtttctaatcagttctctggggtcccagacagattcagtggcagtggggcagggacagatttcacactgaaaatcagcagggtggaagctgaggatgtcgggctttatttctgcatgcaagctacacattttccgatcaccttcggccaagggacacgactggagattaaa

SEQ ID NO: 58 - protein, 112 aa, synthetic construct
  REGION 1..112: note = synthetic
DIVMTQTPLSSPVTLGQPASISCRSSQTLVHGDGNTYLSWIQQRPGQPPRLLIYKVSNQFSGVPDRFSGSGAGTDFTLKISRVEAEDVGLYFCMQATHFPITFGQGTRLEIK

SEQ ID NO: 59 - DNA, 33 bp, synthetic construct
  misc_feature 1..33: note = synthetic
  source 1..33: mol_type = other DNA
caaaccctcgtacacggtgatggaaacacgtac

SEQ ID NO: 60 - protein, 11 aa, synthetic construct
  REGION 1..11: note = synthetic
QTLVHGDGNTY

SEQ ID NO: 61-62 - skipped (2 entries)

SEQ ID NO: 63 - DNA, 27 bp, synthetic construct
  misc_feature 1..27: note = synthetic
  source 1..27: mol_type = other DNA
atgcaagctacacattttccgatcacc

SEQ ID NO: 64 - protein, 9 aa, synthetic construct
  REGION 1..9: note = synthetic
MQATHFPIT

SEQ ID NO: 65 - DNA, 363 bp, synthetic construct
  misc_feature 1..363: note = synthetic
  source 1..363: mol_type = other DNA
caggtacacctggtgcagtctggggctgaggtgaagaagcctggggcctcagtgaaggtctcctgcaaggcttctggatacaccttcaccggctactatatacattgggtgcgacaggcccctggacacgggcttgagtggatgggatggctcaaccctaatactggtaccacaaagtatatacagaactttcagggcagggtcaccatgaccagggacacgtccagcagcacagcctacatggagctgaccaggctgagatctgacgacacggccgtgtattactgtgcgagagatgaggactggaattatgggagctggttcgacacctggggccagggaaccctggtcacagtctcctca

SEQ ID NO: 66 - protein, 121 aa, synthetic construct
  REGION 1..121: note = synthetic
QVHLVQSGAEVKKPGASVKVSCKASGYTFTGYYIHWVRQAPGHGLEWMGWLNPNTGTTKYIQNFQGRVTMTRDTSSSTAYMELTRLRSDDTAVYYCARDEDWNYGSWFDTWGQGTLVTVSS

SEQ ID NO: 67 - DNA, 24 bp, synthetic construct
  misc_feature 1..24: note = synthetic
  source 1..24: mol_type = other DNA
ggatacaccttcaccggctactat

SEQ ID NO: 68 - protein, 8 aa, synthetic construct
  REGION 1..8: note = synthetic
GYTFTGYY

SEQ ID NO: 69 - DNA, 24 bp, synthetic construct
  misc_feature 1..24: note = synthetic
  source 1..24: mol_type = other DNA
ctcaaccctaatactggtaccaca

SEQ ID NO: 70 - protein, 8 aa, synthetic construct
  REGION 1..8: note = synthetic
LNPNTGTT

SEQ ID NO: 71 - DNA, 42 bp, synthetic construct
  misc_feature 1..42: note = synthetic
  source 1..42: mol_type = other DNA
gcgagagatgaggactggaattatgggagctggttcgacacc

SEQ ID NO: 72 - protein, 14 aa, synthetic construct
  REGION 1..14: note = synthetic
ARDEDWNYGSWFDT

SEQ ID NO: 73 - DNA, 336 bp, synthetic construct
  misc_feature 1..336: note = synthetic
  source 1..336: mol_type = other DNA
gatattgtaatgacccagactccactctcctcacctgtcacccttggacagccggcctccatctcctgcaggtctagtccaagcctcgtacacagtgatggaaacacctacttgagttggcttcagcagaggccaggccagcctccaagactcctaatttataagatttctaaccgattctctggggtcccagacagattcagtggcagtggggcagggacagatttcacgctgaaaatcagcagggtggaagctgaggatgtcggggtttattactgcatgcaagctacacattttccgatcaccttcggccaagggacacgactggagattaga

SEQ ID NO: 74 - protein, 112 aa, synthetic construct
  REGION 1..112: note = synthetic
DIVMTQTPLSSPVTLGQPASISCRSSPSLVHSDGNTYLSWLQQRPGQPPRLLIYKISNRFSGVPDRFSGSGAGTDFTLKISRVEAEDVGVYYCMQATHFPITFGQGTRLEIR

SEQ ID NO: 75 - DNA, 33 bp, synthetic construct
  misc_feature 1..33: note = synthetic
  source 1..33: mol_type = other DNA
ccaagcctcgtacacagtgatggaaacacctac

SEQ ID NO: 76 - protein, 11 aa, synthetic construct
  REGION 1..11: note = synthetic
PSLVHSDGNTY

SEQ ID NO: 77-78 - skipped (2 entries)

SEQ ID NO: 79 - DNA, 27 bp, synthetic construct
  misc_feature 1..27: note = synthetic
  source 1..27: mol_type = other DNA
atgcaagctacacattttccgatcacc

SEQ ID NO: 80 - protein, 9 aa, synthetic construct
  REGION 1..9: note = synthetic
MQATHFPIT

SEQ ID NO: 81 - DNA, 360 bp, synthetic construct
  misc_feature 1..360: note = synthetic
  source 1..360: mol_type = other DNA
gaggtgcagctggtggaatctgggggaggtgtggtgcggcctggggggtccctgagactctcctgtgcagcctctggattcacttttgatgattatggcatgacctgggtccgccaagctccagggaggggcctggaatgggtctctggtattcattggcatggtaaacgcacaggttatgcagactctgtgaagggccgattcaccatatccagagacaacgccaagaaatccctgtatctgcaaatgaacagtctgaaaggcgaggacacggccttgtatcattgtgtgagggggggaatgagtacaggggactggttcgacccctggggccagggaaccctggtcatcgtctcctca

SEQ ID NO: 82 - protein, 120 aa, synthetic construct
  REGION 1..120: note = synthetic
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 83 - DNA, 24 bp, synthetic construct
  misc_feature 1..24: note = synthetic
  source 1..24: mol_type = other DNA
ggattcacttttgatgattatggc

SEQ ID NO: 84 - protein, 8 aa, synthetic construct
  REGION 1..8: note = synthetic
GFTFDDYG

SEQ ID NO: 85 - DNA, 24 bp, synthetic construct
  misc_feature 1..24: note = synthetic
  source 1..24: mol_type = other DNA
attcattggcatggtaaacgcaca

SEQ ID NO: 86 - protein, 8 aa, synthetic construct
  REGION 1..8: note = synthetic
IHWHGKRT

SEQ ID NO: 87 - DNA, 39 bp, synthetic construct
  misc_feature 1..39: note = synthetic
  source 1..39: mol_type = other DNA
gtgagggggggaatgagtacaggggactggttcgacccc

SEQ ID NO: 88 - protein, 13 aa, synthetic construct
  REGION 1..13: note = synthetic
VRGGMSTGDWFDP

SEQ ID NO: 89 - DNA, 324 bp, synthetic construct
  misc_feature 1..324: note = synthetic
  source 1..324: mol_type = other DNA
gacatccagatgacccagtctccatcctccctgtctgcatctctaggagacagagtcaccatcacttgccgggcaagtcagagcattaacagttatttaaattggtatcagcagaaaccagggaaagcccctaaactcctgatctatgttgcatccagtttgcaaagtggggtcccatcaaggttcagtggcagtggatctgggacagaattcactctcaccatcagcaatctgcaacctgaagattttgcaacttactactgtcaacagagttacagtacccctccgatcaccttcggccaagggacacgactggagattaaa

SEQ ID NO: 90 - protein, 108 aa, synthetic construct
  REGION 1..108: note = synthetic
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

SEQ ID NO: 91 - DNA, 18 bp, synthetic construct
  misc_feature 1..18: note = synthetic
  source 1..18: mol_type = other DNA
cagagcattaacagttat

SEQ ID NO: 92 - protein, 6 aa, synthetic construct
  REGION 1..6: note = synthetic
QSINSY

SEQ ID NO: 93-94 - skipped (2 entries)

SEQ ID NO: 95 - DNA, 30 bp, synthetic construct
  misc_feature 1..30: note = synthetic
  source 1..30: mol_type = other DNA
caacagagttacagtacccctccgatcacc

SEQ ID NO: 96 - protein, 10 aa, synthetic construct
  REGION 1..10: note = synthetic
QQSYSTPPIT

SEQ ID NO: 97 - DNA, 360 bp, synthetic construct
  misc_feature 1..360: note = synthetic
  source 1..360: mol_type = other DNA
gaggtgcagctggtggagtctgggggaggtgtggtacggccgggggggtccctgagactctcctgtgcagcctctggattcacctttgatgattatggcatgacctgggtccgccaagttccagggaaggggctggagtgggtctctggtattcattggagtggtagaagcacaggttatgcagactctgtgaagggccgattcaccatctccagagacaacgccaagaactccctgtatctgcaaatgaacagtctgagagccgaggacacggccttgtattactgtgcgagggggggaatgagtacgggggactggttcgacccctggggccagggaaccctggtcaccgtctcctca

SEQ ID NO: 98 - protein, 120 aa, synthetic construct
  REGION 1..120: note = synthetic
EVQLVESGGGVVRPGGSLRLSCAASGFTFDDYGMTWVRQVPGKGLEWVSGIHWSGRSTGYADSVKGRFTISRDNAKNSLYLQMNSLRAEDTALYYCARGGMSTGDWFDPWGQGTLVTVSS

SEQ ID NO: 99 - DNA, 24 bp, synthetic construct
  misc_feature 1..24: note = synthetic
  source 1..24: mol_type = other DNA
ggattcacctttgatgattatggc

SEQ ID NO: 100 - protein, 8 aa, synthetic construct
  REGION 1..8: note = synthetic
GFTFDDYG

SEQ ID NO: 101 - DNA, 24 bp, synthetic construct
  misc_feature 1..24: note = synthetic
  source 1..24: mol_type = other DNA
attcattggagtggtagaagcaca

SEQ ID NO: 102 - protein, 8 aa, synthetic construct
  REGION 1..8: note = synthetic
IHWSGRST

SEQ ID NO: 103 - DNA, 39 bp, synthetic construct
  misc_feature 1..39: note = synthetic
  source 1..39: mol_type = other DNA
gcgagggggggaatgagtacgggggactggttcgacccc

SEQ ID NO: 104 - protein, 13 aa, synthetic construct
  REGION 1..13: note = synthetic
ARGGMSTGDWFDP

SEQ ID NO: 105 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgttgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 106 - protein, 108 aa, synthetic construct
  REGION 1..108: note = synthetic
DIQMTQSPSSLSASVGDRVTITCRASQSISSYLNWYQQKPGKAPKLLIYVASSLQSGVPSRFSGSGSGTDFTLTISSLQPEDFATYYCQQSYSTPPITFGQGTRLEIK

SEQ ID NO: 107 - DNA, 18 bp, synthetic construct
  misc_feature 1..18: note = synthetic
  source 1..18: mol_type = other DNA
cagagcattagcagctat

SEQ ID NO: 108 - protein, 6 aa, synthetic construct
  REGION 1..6: note = synthetic
QSISSY

SEQ ID NO: 109-110 - skipped (2 entries)

SEQ ID NO: 111 - DNA, 30 bp, synthetic construct; identical to SEQ ID NO: 95
  misc_feature 1..30: note = synthetic
  source 1..30: mol_type = other DNA

SEQ ID NO: 112 - protein, 10 aa, synthetic construct
  REGION 1..10: note = synthetic
QQSYSTPPIT

SEQ ID NO: 113 - DNA, 345 bp, synthetic construct
  misc_feature 1..345: note = synthetic
  source 1..345: mol_type = other DNA
gaggtgcagttggtggagtctggaggaggcttggtccagcctggggggtccctaagactctcctgtgcagcctctgggttcaccgtcggtagtaactacatgaactgggtccgtcaggctccagggaagggactggagtgggtctcagttatttatagtggtggtagtacatactacgcagattccgtgaagggccgattcaccatctccagactcacttccaagaacacactgtatcttcaaatgagcagcctgagacctgaggacacggccgtgtattattgtgcgagagggattaggggtctggacgtctggggccaagggaccacggtcaccgtctcttca

SEQ ID NO: 114 - protein, 115 aa, synthetic construct
  REGION 1..115: note = synthetic
EVQLVESGGGLVQPGGSLRLSCAASGFTVGSNYMNWVRQAPGKGLEWVSVIYSGGSTYYADSVKGRFTISRLTSKNTLYLQMSSLRPEDTAVYYCARGIRGLDVWGQGTTVTVSS

SEQ ID NO: 115 - DNA, 24 bp, synthetic construct
  misc_feature 1..24: note = synthetic
  source 1..24: mol_type = other DNA
gggttcaccgtcggtagtaactac

SEQ ID NO: 116 - protein, 8 aa, synthetic construct
  REGION 1..8: note = synthetic
GFTVGSNY

SEQ ID NO: 117 - DNA, 21 bp, synthetic construct
  misc_feature 1..21: note = synthetic
  source 1..21: mol_type = other DNA
atttatagtggtggtagtaca

SEQ ID NO: 118 - protein, 7 aa, synthetic construct
  REGION 1..7: note = synthetic
IYSGGST

SEQ ID NO: 119 - DNA, 27 bp, synthetic construct
  misc_feature 1..27: note = synthetic
  source 1..27: mol_type = other DNA
gcgagagggattaggggtctggacgtc

SEQ ID NO: 120 - protein, 9 aa, synthetic construct
  REGION 1..9: note = synthetic
ARGIRGLDV

SEQ ID NO: 121 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagaccattaacatctatttaaattggtatcagcagaaaccagggagagcccctaggctcctgatctatgctgcatccagtttgcaaagtggggtcccatcaaggttcagtggcagtggatctgggacagatttcactctcaccatcagcagtctgcaacctgaagattttgcaacttactactgtcaccagagttacagtacccctccgatcaccttcggccaagggacacgactggagattaaa

SEQ ID NO: 122 - protein, 108 aa, synthetic construct
  REGION 1..108: note = synthetic
DIQMTQSPSSLSASVGDRVTITCRASQTINIYLNWYQQKPGRAPRLLIYAASSLQSGVPSRFSGSGSGTDFTLTISSLQPEDFATYYCHQSYSTPPITFGQGTRLEIK

SEQ ID NO: 123 - DNA, 18 bp, synthetic construct
  misc_feature 1..18: note = synthetic
  source 1..18: mol_type = other DNA
cagaccattaacatctat

SEQ ID NO: 124 - protein, 6 aa, synthetic construct
  REGION 1..6: note = synthetic
QTINIY

SEQ ID NO: 125-126 - skipped (2 entries)

SEQ ID NO: 127 - DNA, 30 bp, synthetic construct
  misc_feature 1..30: note = synthetic
  source 1..30: mol_type = other DNA
caccagagttacagtacccctccgatcacc

SEQ ID NO: 128 - protein, 10 aa, synthetic construct
  REGION 1..10: note = synthetic
HQSYSTPPIT

SEQ ID NO: 129 - DNA, 345 bp, synthetic construct
  misc_feature 1..345: note = synthetic
  source 1..345: mol_type = other DNA
gaggaacggttggtggagtctggaggagacttggtccagcctggggggtccctgagactctcctgtgcagcctctggcatcaccgtcggtactaattatatgaactgggtccgccaggctccagggaagggactggagtgggtctcagttatttctagcggtggtaatacacactacgcagactccgtgaagggccgattcattatgtccagacaaacttccaaaaacacgctgtatcttcagatgaatagcctggaaactgaggacacggccgtatattattgtgcgagggggatcagaggtttggacgtctggggccaagggaccatggtcaccgtctcctca

SEQ ID NO: 130 - protein, 115 aa, synthetic construct
  REGION 1..115: note = synthetic
EERLVESGGDLVQPGGSLRLSCAASGITVGTNYMNWVRQAPGKGLEWVSVISSGGNTHYADSVKGRFIMSRQTSKNTLYLQMNSLETEDTAVYYCARGIRGLDVWGQGTMVTVSS

SEQ ID NO: 131 - DNA, 24 bp, synthetic construct
  misc_feature 1..24: note = synthetic
  source 1..24: mol_type = other DNA
ggcatcaccgtcggtactaattat

SEQ ID NO: 132 - protein, 8 aa, synthetic construct
  REGION 1..8: note = synthetic
GITVGTNY

SEQ ID NO: 133 - DNA, 21 bp, synthetic construct
  misc_feature 1..21: note = synthetic
  source 1..21: mol_type = other DNA
atttctagcggtggtaataca

SEQ ID NO: 134 - protein, 7 aa, synthetic construct
  REGION 1..7: note = synthetic
ISSGGNT

SEQ ID NO: 135 - DNA, 27 bp, synthetic construct
  misc_feature 1..27: note = synthetic
  source 1..27: mol_type = other DNA
gcgagggggatcagaggtttggacgtc

SEQ ID NO: 136 - protein, 9 aa, synthetic construct
  REGION 1..9: note = synthetic
ARGIRGLDV

SEQ ID NO: 137 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcatgagcagctatttaaattggtatcagcagaaaccagggagagcccctaagctcctgatctttgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 138 - protein, 108 aa, synthetic construct
  REGION 1..108: note = synthetic
DIQMTQSPSSLSASVGDRVTITCRASQSMSSYLNWYQQKPGRAPKLLIFAASSLQSGVPSRFSGSGSGTDFTLTISSLQPEDFATYYCQQSYSTPPITFGQGTRLEIK

SEQ ID NO: 139 - DNA, 18 bp, synthetic construct
  misc_feature 1..18: note = synthetic
  source 1..18: mol_type = other DNA
cagagcatgagcagctat

SEQ ID NO: 140 - protein, 6 aa, synthetic construct
  REGION 1..6: note = synthetic
QSMSSY

SEQ ID NO: 141-142 - skipped (2 entries)

SEQ ID NO: 143 - DNA, 30 bp, synthetic construct; identical to SEQ ID NO: 95
  misc_feature 1..30: note = synthetic
  source 1..30: mol_type = other DNA

SEQ ID NO: 144 - protein, 10 aa, synthetic construct
  REGION 1..10: note = synthetic
QQSYSTPPIT

SEQ ID NO: 145 - DNA, 354 bp, synthetic construct
  misc_feature 1..354: note = synthetic
  source 1..354: mol_type = other DNA
caggtccagctggtgcagtctggggctgaggtgaagatgcctgggtcctcggtgagggtctcctgcaaggcttctggaggcatcttcagcagttctactatcagttgggtgcgacaggcccctggacaagggcttgaatggatgggagagatcatccctgtctttggtacagtaaactacgcacagaagttccaggacagagtcatatttaccgcggacgaatctacgactacagcctacatggagctgagcagcctgaaatctggggacacggccgtatatttctgtgcgcgaaattggggattaggctctttttatatctggggccaagggacaatggtcaccgtctcttca

SEQ ID NO: 146 - protein, 118 aa, synthetic construct
  REGION 1..118: note = synthetic
QVQLVQSGAEVKMPGSSVRVSCKASGGIFSSSTISWVRQAPGQGLEWMGEIIPVFGTVNYAQKFQDRVIFTADESTTTAYMELSSLKSGDTAVYFCARNWGLGSFYIWGQGTMVTVSS

SEQ ID NO: 147 - DNA, 24 bp, synthetic construct
  misc_feature 1..24: note = synthetic
  source 1..24: mol_type = other DNA
ggaggcatcttcagcagttctact

SEQ ID NO: 148 - protein, 8 aa, synthetic construct
  REGION 1..8: note = synthetic
GGIFSSST

SEQ ID NO: 149 - DNA, 24 bp, synthetic construct
  misc_feature 1..24: note = synthetic
  source 1..24: mol_type = other DNA
atcatccctgtctttggtacagta

SEQ ID NO: 150 - protein, 8 aa, synthetic construct
  REGION 1..8: note = synthetic
IIPVFGTV

SEQ ID NO: 151 - DNA, 33 bp, synthetic construct
  misc_feature 1..33: note = synthetic
  source 1..33: mol_type = other DNA
gcgcgaaattggggattaggctctttttatatc

SEQ ID NO: 152 - protein, 11 aa, synthetic construct
  REGION 1..11: note = synthetic
ARNWGLGSFYI

SEQ ID NO: 153 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagagttttaacttcaactacttagcctggtaccagcagaaacctggccaggctcccagactcctcatctatggtgcatccagcagggccactggcatcccagacaggttcagtggcagtgggtctgggacagacttcactctcaccatcaacaggctggagcctgaagattttggagtgttttattgtcagcagtatgaaagcgcaccttggacgttcggccaagggaccaaggtggaaatcaaa

SEQ ID NO: 154 - protein, 108 aa, synthetic construct
  REGION 1..108: note = synthetic
EIVLTQSPGTLSLSPGERATLSCRASQSFNFNYLAWYQQKPGQAPRLLIYGASSRATGIPDRFSGSGSGTDFTLTINRLEPEDFGVFYCQQYESAPWTFGQGTKVEIK

SEQ ID NO: 155 - DNA, 21 bp, synthetic construct
  misc_feature 1..21: note = synthetic
  source 1..21: mol_type = other DNA
cagagttttaacttcaactac

SEQ ID NO: 156 - protein, 7 aa, synthetic construct
  REGION 1..7: note = synthetic
QSFNFNY

SEQ ID NO: 157-158 - skipped (2 entries)

SEQ ID NO: 159 - DNA, 27 bp, synthetic construct
  misc_feature 1..27: note = synthetic
  source 1..27: mol_type = other DNA
cagcagtatgaaagcgcaccttggacg

SEQ ID NO: 160 - protein, 9 aa, synthetic construct
  REGION 1..9: note = synthetic
QQYESAPWT

SEQ ID NO: 161 - DNA, 345 bp, synthetic construct
  misc_feature 1..345: note = synthetic
  source 1..345: mol_type = other DNA
gaggtgcagcttgtagagtctgggggagacttggtacatcctggcaggtccctgagactctcctgtgcagcctctggtttcccctttgatgagtatgccatgcactgggtccggcaagttccagggaagggcctggagtgggtctcaggtattagttggagtaataataacataggctatgcggactctgtgaagggccgattcaccatctccagagacaacgccaaaaactccctgtatctacaaatgaacagtctgagacctgaggacacggccttttattactgtgcaaaatctggaatctttgactcctggggccagggaaccctggtcaccgtctcctca

SEQ ID NO: 162 - protein, 115 aa, synthetic construct
  REGION 1..115: note = synthetic
EVQLVESGGDLVHPGRSLRLSCAASGFPFDEYAMHWVRQVPGKGLEWVSGISWSNNNIGYADSVKGRFTISRDNAKNSLYLQMNSLRPEDTAFYYCAKSGIFDSWGQGTLVTVSS

SEQ ID NO: 163 - DNA, 24 bp, synthetic construct
  misc_feature 1..24: note = synthetic
  source 1..24: mol_type = other DNA
ggtttcccctttgatgagtatgcc

SEQ ID NO: 164 - protein, 8 aa, synthetic construct
  REGION 1..8: note = synthetic
GFPFDEYA

SEQ ID NO: 165 - DNA, 24 bp, synthetic construct
  misc_feature 1..24: note = synthetic
  source 1..24: mol_type = other DNA
attagttggagtaataataacata

SEQ ID NO: 166 - protein, 8 aa, synthetic construct
  REGION 1..8: note = synthetic
ISWSNNNI

SEQ ID NO: 167 - DNA, 24 bp, synthetic construct
  misc_feature 1..24: note = synthetic
  source 1..24: mol_type = other DNA
gcaaaatctggaatctttgactcc

SEQ ID NO: 168 - protein, 8 aa, synthetic construct
  REGION 1..8: note = synthetic
AKSGIFDS

SEQ ID NO: 169 - DNA, 315 bp, synthetic construct
  misc_feature 1..315: note = synthetic
  source 1..315: mol_type = other DNA
gacatccagatgacccagtctccatcctccctgtctgcatctgtaggagacagagtcaccatcacttgccgggcaagtcagagcattagcagctatttaaattggtatcagcagaaaccagggaagctcctgatctatgctgcatccagtttgcaaagtggggtcccatcacggttcagtggcggtggatctgggacagatttcactctcaccatcagcagtctgcgacctgaagattttgcaacttactactgtcaacagagttactgtacccctccgatcaccttcggccaagggacacgactggagattaaa

SEQ ID NO: 170 - protein, 105 aa, synthetic construct
  REGION 1..105: note = synthetic
DIQMTQSPSSLSASVGDRVTITCRASQSISSYLNWYQQKPGKLLIYAASSLQSGVPSRFSGGGSGTDFTLTISSLRPEDFATYYCQQSYCTPPITFGQGTRLEIK

SEQ ID NO: 171 - DNA, 18 bp, synthetic construct
  misc_feature 1..18: note = synthetic
  source 1..18: mol_type = other DNA
cagagcattagcagctat

SEQ ID NO: 172 - protein, 6 aa, synthetic construct
  REGION 1..6: note = synthetic
QSISSY

SEQ ID NO: 173-174 - skipped (2 entries)

SEQ ID NO: 175 - DNA, 30 bp, synthetic construct
  misc_feature 1..30: note = synthetic
  source 1..30: mol_type = other DNA
caacagagttactgtacccctccgatcacc

SEQ ID NO: 176 - protein, 10 aa, synthetic construct
  REGION 1..10: note = synthetic
QQSYCTPPIT

SEQ ID NO: 177 - DNA, 354 bp, synthetic construct
  misc_feature 1..354: note = synthetic
  source 1..354: mol_type = other DNA
gaggtgcagctggtggagtccgggggaggcgtggtccagcctgggaggtccctgagactctcctgtgcagcctctggattcaccttcagtagttatggcatgcactgggtccgccaggctccaggcaagggactggagtgggtgacacttatatcatatgagggaaggaataaatactatgcagactccgtgaagggccgattcaccatttccagagacaattccaagaacacgctgtatctgcaaatgaacagcctgagagctgaggacacggctgtatattactgtgcgaaagataggaccctttacggtatggacgtctggggccaaggaaccacggtcaccgtctcctca

SEQ ID NO: 178 - protein, 118 aa, synthetic construct
  REGION 1..118: note = synthetic
EVQLVESGGGVVQPGRSLRLSCAASGFTFSSYGMHWVRQAPGKGLEWVTLISYEGRNKYYADSVKGRFTISRDNSKNTLYLQMNSLRAEDTAVYYCAKDRTLYGMDVWGQGTTVTVSS

SEQ ID NO: 179 - DNA, 24 bp, synthetic construct
  misc_feature 1..24: note = synthetic
  source 1..24: mol_type = other DNA
ggattcaccttcagtagttatggc

SEQ ID NO: 180 - protein, 8 aa, synthetic construct
  REGION 1..8: note = synthetic
GFTFSSYG

SEQ ID NO: 181 - DNA, 24 bp, synthetic construct
  misc_feature 1..24: note = synthetic
  source 1..24: mol_type = other DNA
atatcatatgagggaaggaataaa

SEQ ID NO: 182 - protein, 8 aa, synthetic construct
  REGION 1..8: note = synthetic
ISYEGRNK

SEQ ID NO: 183 - DNA, 33 bp, synthetic construct
  misc_feature 1..33: note = synthetic
  source 1..33: mol_type = other DNA
gcgaaagataggaccctttacggtatggacgtc

SEQ ID NO: 184 - protein, 11 aa, synthetic construct
  REGION 1..11: note = synthetic
AKDRTLYGMDV

SEQ ID NO: 185 - DNA, 363 bp, synthetic construct
  misc_feature 1..363: note = synthetic
  source 1..363: mol_type = other DNA
caggtcaccttgagggagtctggtcctgcgctggtgaaaaccacacagaccctcacactgacctgcaccttctctgggttctcactcagcactaatagaatgtgtgtgacctggatccgtcagcccccagggaaggccctggagtggcttgcgcgcattgattgggatggtgttaaatactacaacacatctctgaagaccaggctcaccatctccaaggacacctccaaaaaccaggtggtccttacaatgaccaacatggaccctgtggacacagccactttttactgtgcacggtcgacttcgttgactttttactactttgactactggggccagggaaccctggtcaccgtctcctca

SEQ ID NO: 186 - protein, 121 aa, synthetic construct
  REGION 1..121: note = synthetic
QVTLRESGPALVKTTQTLTLTCTFSGFSLSTNRMCVTWIRQPPGKALEWLARIDWDGVKYYNTSLKTRLTISKDTSKNQVVLTMTNMDPVDTATFYCARSTSLTFYYFDYWGQGTLVTVSS

SEQ ID NO: 187 - DNA, 30 bp, synthetic construct
  misc_feature 1..30: note = synthetic
  source 1..30: mol_type = other DNA
gggttctcactcagcactaatagaatgtgt

SEQ ID NO: 188 - protein, 10 aa, synthetic construct
  REGION 1..10: note = synthetic
GFSLSTNRMC

SEQ ID NO: 189 - DNA, 21 bp, synthetic construct
  misc_feature 1..21: note = synthetic
  source 1..21: mol_type = other DNA
attgattgggatggtgttaaa

SEQ ID NO: 190 - protein, 7 aa, synthetic construct
  REGION 1..7: note = synthetic
IDWDGVK

SEQ ID NO: 191 - DNA, 39 bp, synthetic construct
  misc_feature 1..39: note = synthetic
  source 1..39: mol_type = other DNA
gcacggtcgacttcgttgactttttactactttgactac

SEQ ID NO: 192 - protein, 13 aa, synthetic construct
  REGION 1..13: note = synthetic
ARSTSLTFYYFDY

SEQ ID NO: 193 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 194 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 195 - DNA, 18 bp, synthetic construct
  misc_feature 1..18: note = synthetic
  source 1..18: mol_type = other DNA
cagagcattagcagctat

SEQ ID NO: 196 - protein, 6 aa, synthetic construct
  REGION 1..6: note = synthetic
QSISSY

SEQ ID NO: 197-198 - skipped (2 entries)

SEQ ID NO: 199 - DNA, 30 bp, synthetic construct; identical to SEQ ID NO: 95
  misc_feature 1..30: note = synthetic
  source 1..30: mol_type = other DNA

SEQ ID NO: 200 - protein, 10 aa, synthetic construct
  REGION 1..10: note = synthetic
QQSYSTPPIT

SEQ ID NO: 201 - DNA, 345 bp, synthetic construct
  misc_feature 1..345: note = synthetic
  source 1..345: mol_type = other DNA
gaggtgcagctggtggagtctggaggaggcttggtccagccgggggggtccctgagactctcctgtgcagcctctgagttcaccgtcggtaccaaccacatgaactgggtccgccaggctccagggaagggactggagtgggtctcagttatttatagcggtggtaacacattctacgcagactccgtgaagggccgattcaccatctccagacacacttccaagaacacgctgtatcttcaaatgaacagcctgacagcagaggacacggccgtatattactgtgcgcgaggattggggggtatggacgtctggggccaagggaccacggtcaccgtctcctca

SEQ ID NO: 202 - protein, 115 aa, synthetic construct
  REGION 1..115: note = synthetic
EVQLVESGGGLVQPGGSLRLSCAASEFTVGTNHMNWVRQAPGKGLEWVSVIYSGGNTFYADSVKGRFTISRHTSKNTLYLQMNSLTAEDTAVYYCARGLGGMDVWGQGTTVTVSS

SEQ ID NO: 203 - DNA, 24 bp, synthetic construct
  misc_feature 1..24: note = synthetic
  source 1..24: mol_type = other DNA
gagttcaccgtcggtaccaaccac

SEQ ID NO: 204 - protein, 8 aa, synthetic construct
  REGION 1..8: note = synthetic
EFTVGTNH

SEQ ID NO: 205 - DNA, 21 bp, synthetic construct
  misc_feature 1..21: note = synthetic
  source 1..21: mol_type = other DNA
atttatagcggtggtaacaca

SEQ ID NO: 206 - protein, 7 aa, synthetic construct
  REGION 1..7: note = synthetic
IYSGGNT

SEQ ID NO: 207 - DNA, 27 bp, synthetic construct
  misc_feature 1..27: note = synthetic
  source 1..27: mol_type = other DNA
gcgcgaggattggggggtatggacgtc

SEQ ID NO: 208 - protein, 9 aa, synthetic construct
  REGION 1..9: note = synthetic
ARGLGGMDV

SEQ ID NO: 209 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcgagtcaggtcattagcaattatttagcctggtatcagcagaaaccagggaaagttcctaggctcctgatctatgctgcatccactttgcaatcaggggtcccatctcgtttcagtggcagtggatctgggacagatttcactctcaccatcagcagcctgcagcctgaagatgttgcaacttattactgtcaaaagtataacagtgcccctcggacgttcggccaagggaccaaggtggaaatcaaa

SEQ ID NO: 210 - protein, 107 aa, synthetic construct
  REGION 1..107: note = synthetic
DIQMTQSPSSLSASVGDRVTITCRASQVISNYLAWYQQKPGKVPRLLIYAASTLQSGVPSRFSGSGSGTDFTLTISSLQPEDVATYYCQKYNSAPRTFGQGTKVEIK

SEQ ID NO: 211 - DNA, 18 bp, synthetic construct
  misc_feature 1..18: note = synthetic
  source 1..18: mol_type = other DNA
caggtcattagcaattat

SEQ ID NO: 212 - protein, 6 aa, synthetic construct
  REGION 1..6: note = synthetic
QVISNY

SEQ ID NO: 213-214 - skipped (2 entries)

SEQ ID NO: 215 - DNA, 27 bp, synthetic construct
  misc_feature 1..27: note = synthetic
  source 1..27: mol_type = other DNA
caaaagtataacagtgcccctcggacg

SEQ ID NO: 216 - protein, 9 aa, synthetic construct
  REGION 1..9: note = synthetic
QKYNSAPRT

SEQ ID NO: 217 - DNA, 360 bp, synthetic construct
  misc_feature 1..360: note = synthetic
  source 1..360: mol_type = other DNA
gaggtgcagctggtggagtctgggggaggcttggtccagcggggggagtccctgagactttactgtgcagcctctggattcacctttagtaaatattggatgaactgggtccgccaggctccagggaaggggctggagtgggtggccaacataaagggagatggaagtgagaaatactatgtggactctgtgaagggccggttcaccatctccagagacaacgccaagaactcactatatctacaaatgaacagcctgagagccgaggacacggctgtttattactgtgcgagagattattggggatcaggctactactttgacttctggggccagggaaccctggtcaccgtctcctca

SEQ ID NO: 218 - protein, 120 aa, synthetic construct
  REGION 1..120: note = synthetic
EVQLVESGGGLVQRGESLRLYCAASGFTFSKYWMNWVRQAPGKGLEWVANIKGDGSEKYYVDSVKGRFTISRDNAKNSLYLQMNSLRAEDTAVYYCARDYWGSGYYFDFWGQGTLVTVSS

SEQ ID NO: 219 - DNA, 24 bp, synthetic construct
  misc_feature 1..24: note = synthetic
  source 1..24: mol_type = other DNA
ggattcacctttagtaaatattgg

SEQ ID NO: 220 - protein, 8 aa, synthetic construct
  REGION 1..8: note = synthetic
GFTFSKYW

SEQ ID NO: 221 - DNA, 24 bp, synthetic construct
  misc_feature 1..24: note = synthetic
  source 1..24: mol_type = other DNA
ataaagggagatggaagtgagaaa

SEQ ID NO: 222 - protein, 8 aa, synthetic construct
  REGION 1..8: note = synthetic
IKGDGSEK

SEQ ID NO: 223 - DNA, 39 bp, synthetic construct
  misc_feature 1..39: note = synthetic
  source 1..39: mol_type = other DNA
gcgagagattattggggatcaggctactactttgacttc

SEQ ID NO: 224 - protein, 13 aa, synthetic construct
  REGION 1..13: note = synthetic
ARDYWGSGYYFDF

SEQ ID NO: 225 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagaacattaacaactatttaaattggtatcagcagaaaccagggaaagcccctaaactcctgatctatgctgcatccagtttccaaaatgcggtcccatcaaggttcagtggcagtggatctgggacagatttcactctcaccatcagcagtctgcaacctgaagattttgcaacttactactgtcaacagagttacaataccccgctcactttcggcggggggaccaaggtggagatcaaa

SEQ ID NO: 226 - protein, 107 aa, synthetic construct
  REGION 1..107: note = synthetic
DIQMTQSPSSLSASVGDRVTITCRASQNINNYLNWYQQKPGKAPKLLIYAASSFQNAVPSRFSGSGSGTDFTLTISSLQPEDFATYYCQQSYNTPLTFGGGTKVEIK

SEQ ID NO: 227 - DNA, 18 bp, synthetic construct
  misc_feature 1..18: note = synthetic
  source 1..18: mol_type = other DNA
cagaacattaacaactat

SEQ ID NO: 228 - protein, 6 aa, synthetic construct
  REGION 1..6: note = synthetic
QNINNY

SEQ ID NO: 229-230 - skipped (2 entries)

SEQ ID NO: 231 - DNA, 27 bp, synthetic construct
  misc_feature 1..27: note = synthetic
  source 1..27: mol_type = other DNA
caacagagttacaataccccgctcact

SEQ ID NO: 232 - protein, 9 aa, synthetic construct
  REGION 1..9: note = synthetic
QQSYNTPLT

SEQ ID NO: 233 - DNA, 390 bp, synthetic construct
  misc_feature 1..390: note = synthetic
  source 1..390: mol_type = other DNA
gaggtgcagctggtggagtctgggggaggcttggtccagtctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgatgacacggctgtgtattactgtgcgagagatgatattgtagtagtaccagctcctatgggatattactactactacttcggtatggacgtctggggccaagggaccacggtcaccgtctcctca

SEQ ID NO: 234 - protein, 130 aa, synthetic construct
  REGION 1..130: note = synthetic
EVQLVESGGGLVQSGGSLRLSCAASGFTFSSYWMSWVRQAPGKGLEWVANIKQDGSEKYYVDSVKGRFTISRDNAKNSLYLQMNSLRADDTAVYYCARDDIVVVPAPMGYYYYYFGMDVWGQGTTVTVSS

SEQ ID NO: 235 - DNA, 24 bp, synthetic construct
  misc_feature 1..24: note = synthetic
  source 1..24: mol_type = other DNA
ggattcacctttagtagctattgg

SEQ ID NO: 236 - protein, 8 aa, synthetic construct
  REGION 1..8: note = synthetic
GFTFSSYW

SEQ ID NO: 237 - DNA, 24 bp, synthetic construct
  misc_feature 1..24: note = synthetic
  source 1..24: mol_type = other DNA
ataaagcaagatggaagtgagaaa

SEQ ID NO: 238 - protein, 8 aa, synthetic construct
  REGION 1..8: note = synthetic
IKQDGSEK

SEQ ID NO: 239 - DNA, 69 bp, synthetic construct
  misc_feature 1..69: note = synthetic
  source 1..69: mol_type = other DNA
gcgagagatgatattgtagtagtaccagctcctatgggatattactactactacttcggtatggacgtc

SEQ ID NO: 240 - protein, 23 aa, synthetic construct
  REGION 1..23: note = synthetic
ARDDIVVVPAPMGYYYYYFGMDV

SEQ ID NO: 241 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 242 - protein, 107 aa, synthetic construct
  REGION 1..107: note = synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 243 - DNA, 18 bp, synthetic construct
  misc_feature 1..18: note = synthetic
  source 1..18: mol_type = other DNA
cagggcattagaaatgat

SEQ ID NO: 244 - protein, 6 aa, synthetic construct
  REGION 1..6: note = synthetic
QGIRND

SEQ ID NO: 245-246 - skipped (2 entries)

SEQ ID NO: 247 - DNA, 27 bp, synthetic construct
  misc_feature 1..27: note = synthetic
  source 1..27: mol_type = other DNA
ctacagcataatagttacccgtacact

SEQ ID NO: 248 - protein, 9 aa, synthetic construct
  REGION 1..9: note = synthetic
LQHNSYPYT

SEQ ID NO: 249 - DNA, 369 bp, synthetic construct
  misc_feature 1..369: note = synthetic
  source 1..369: mol_type = other DNA
gaagtgcagctggtggagtctgggggaggcttggttcagcctggcaggtccctgagactctcctgtgcagcctctggattcacctttgatgattttgccatgcactgggtccgacaagctccagggaagggcctggagtgggtctcaggtattagttggactggtggtaacatggactatgcgaactctgtgaagggccgattcaccatctccagagaggacgccaagaattccctgtatctgcaaatgaacagtctgagagctgcggacacggccttgtattactgtgtaaaagatataagggggatagtggctacggggggggcttttgatatctggggccgagggacaatggtcaccgtctcttca

SEQ ID NO: 250 - protein, 123 aa, synthetic construct
  REGION 1..123: note = synthetic
EVQLVESGGGLVQPGRSLRLSCAASGFTFDDFAMHWVRQAPGKGLEWVSGISWTGGNMDYANSVKGRFTISREDAKNSLYLQMNSLRAADTALYYCVKDIRGIVATGGAFDIWGRGTMVTVSS

SEQ ID NO: 251 - DNA, 24 bp, synthetic construct
  misc_feature 1..24: note = synthetic
  source 1..24: mol_type = other DNA
ggattcacctttgatgattttgcc

SEQ ID NO: 252 - protein, 8 aa, synthetic construct
  REGION 1..8: note = synthetic
GFTFDDFA

SEQ ID NO: 253 - DNA, 24 bp, synthetic construct
  misc_feature 1..24: note = synthetic
  source 1..24: mol_type = other DNA
attagttggactggtggtaacatg

SEQ ID NO: 254 - protein, 8 aa, synthetic construct
  REGION 1..8: note = synthetic
ISWTGGNM

SEQ ID NO: 255 - DNA, 48 bp, synthetic construct
  misc_feature 1..48: note = synthetic
  source 1..48: mol_type = other DNA
gtaaaagatataagggggatagtggctacggggggggcttttgatatc

SEQ ID NO: 256 - protein, 16 aa, synthetic construct
  REGION 1..16: note = synthetic
VKDIRGIVATGGAFDI

SEQ ID NO: 257 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatctcttgccgggcaagtcagaccattagcacttatttaaattggtttcagcagaaaccagggaaagcccctaagctcctgatctatgttgtgtccagtttgcaaagtggggtcccatcaaggttcagtggcagtggatctgggacagatttcactctcaccatcagcagtctgcaacctgaagattttgcaacttattactgtcaacagagttacagtaccccattcactttcggccctgggaccaaagtggatatcaaa

SEQ ID NO: 258 - protein, 107 aa, synthetic construct
  REGION 1..107: note = synthetic
DIQMTQSPSSLSASVGDRVTISCRASQTISTYLNWFQQKPGKAPKLLIYVVSSLQSGVPSRFSGSGSGTDFTLTISSLQPEDFATYYCQQSYSTPFTFGPGTKVDIK

SEQ ID NO: 259 - DNA, 18 bp, synthetic construct
  misc_feature 1..18: note = synthetic
  source 1..18: mol_type = other DNA
cagaccattagcacttat

SEQ ID NO: 260 - protein, 6 aa, synthetic construct
  REGION 1..6: note = synthetic
QTISTY

SEQ ID NO: 261-262 - skipped (2 entries)

SEQ ID NO: 263 - DNA, 27 bp, synthetic construct
  misc_feature 1..27: note = synthetic
  source 1..27: mol_type = other DNA
caacagagttacagtaccccattcact

SEQ ID NO: 264 - protein, 9 aa, synthetic construct
  REGION 1..9: note = synthetic
QQSYSTPFT

SEQ ID NO: 265 - DNA, 345 bp, synthetic construct
  misc_feature 1..345: note = synthetic
  source 1..345: mol_type = other DNA
gaggtgcagctggtggagtctggaggaggcttggtccagccgggggggtccctgagactctcctgtgcagcctctggattcaccgtcggtaccaactacatgaactgggtccgccaggctccagggaagggactggagtggatctcagttatttatagcggtggtagcacattctacgcagactccgtgaagggccgattcaccatctccagacagacttcccagaacacgctgtatcttcaaatgaacagcctgagacctgaggacacggccgtatattactgtgcgagaggtatacgtggttttgatatctggggccaagggacaatggtcaccgtctcttca

SEQ ID NO: 266 - protein, 115 aa, synthetic construct
  REGION 1..115: note = synthetic
EVQLVESGGGLVQPGGSLRLSCAASGFTVGTNYMNWVRQAPGKGLEWISVIYSGGSTFYADSVKGRFTISRQTSQNTLYLQMNSLRPEDTAVYYCARGIRGFDIWGQGTMVTVSS

SEQ ID NO: 267 - DNA, 24 bp, synthetic construct
  misc_feature 1..24: note = synthetic
  source 1..24: mol_type = other DNA
ggattcaccgtcggtaccaactac

SEQ ID NO: 268 - protein, 8 aa, synthetic construct
  REGION 1..8: note = synthetic
GFTVGTNY

SEQ ID NO: 269 - DNA, 21 bp, synthetic construct
  misc_feature 1..21: note = synthetic
  source 1..21: mol_type = other DNA
atttatagcggtggtagcaca

SEQ ID NO: 270 - protein, 7 aa, synthetic construct
  REGION 1..7: note = synthetic
IYSGGST

SEQ ID NO: 271 - DNA, 27 bp, synthetic construct
  misc_feature 1..27: note = synthetic
  source 1..27: mol_type = other DNA
gcgagaggtatacgtggttttgatatc

SEQ ID NO: 272 - protein, 9 aa, synthetic construct
  REGION 1..9: note = synthetic
ARGIRGFDI

SEQ ID NO: 273 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 274 - protein, 108 aa, synthetic construct; identical to SEQ ID NO: 194
  REGION 1..108: note = synthetic

SEQ ID NO: 275 - DNA, 18 bp, synthetic construct
  misc_feature 1..18: note = synthetic
  source 1..18: mol_type = other DNA
cagagcattagcagctat

SEQ ID NO: 276 - protein, 6 aa, synthetic construct
  REGION 1..6: note = synthetic
QSISSY

SEQ ID NO: 277-278 - skipped (2 entries)

SEQ ID NO: 279 - DNA, 30 bp, synthetic construct; identical to SEQ ID NO: 95
  misc_feature 1..30: note = synthetic
  source 1..30: mol_type = other DNA

SEQ ID NO: 280 - protein, 10 aa, synthetic construct
  REGION 1..10: note = synthetic
QQSYSTPPIT

SEQ ID NO: 281 - DNA, 345 bp, synthetic construct
  misc_feature 1..345: note = synthetic
  source 1..345: mol_type = other DNA
gaggtgcagctggtggagtctggaggaggcttggtccagccgggggggtccctgagactctcctgtgcagcctctgggtttaccatcagtaccaactacatgaactgggtccgccaggctccagggaaggggctggagtgggtcgcagttatttatagcagtggttccacatactatatcgactccgtgaagggccgattcaccatctccagactcacttccaagaacacggtgtatcttcaaatgagcagcctgaattctgaagacacggccgtgtattactgtgcgagggggatcaggggttttgatatttggggccaagggacaatggtcaccgtctcttca

SEQ ID NO: 282 - protein, 115 aa, synthetic construct
  REGION 1..115: note = synthetic
EVQLVESGGGLVQPGGSLRLSCAASGFTISTNYMNWVRQAPGKGLEWVAVIYSSGSTYYIDSVKGRFTISRLTSKNTVYLQMSSLNSEDTAVYYCARGIRGFDIWGQGTMVTVSS

SEQ ID NO: 283 - DNA, 24 bp, synthetic construct
  misc_feature 1..24: note = synthetic
  source 1..24: mol_type = other DNA
gggtttaccatcagtaccaactac

SEQ ID NO: 284 - protein, 8 aa, synthetic construct
  REGION 1..8: note = synthetic
GFTISTNY

SEQ ID NO: 285 - DNA, 21 bp, synthetic construct
  misc_feature 1..21: note = synthetic
  source 1..21: mol_type = other DNA
atttatagcagtggttccaca

SEQ ID NO: 286 - protein, 7 aa, synthetic construct
  REGION 1..7: note = synthetic
IYSSGST

SEQ ID NO: 287 - DNA, 27 bp, synthetic construct
  misc_feature 1..27: note = synthetic
  source 1..27: mol_type = other DNA
gcgagggggatcaggggttttgatatt

SEQ ID NO: 288 - protein, 9 aa, synthetic construct
  REGION 1..9: note = synthetic
ARGIRGFDI

SEQ ID NO: 289 - DNA, 372 bp, synthetic construct
  misc_feature 1..372: note = synthetic
  source 1..372: mol_type = other DNA
gaagtgcagctggtggagtcggggggaggcttggtacagcctggcaggtccctgagactctcctgtgcagcctctggattcaccattgatgatagtgccatgcactgggtccggcaaactccagggaagggcctggagtgggtctcaggtattagttggaaaagtggtagcataggttatgcggactctgtgaggggccgattcaccatctccagagacaacgccaagaattccctctatctgcaaatgaacagtctgagagttgaggacacggccttgtattactgtgtaaaagatataaggggcaactggaactacgggggaaactggttcgacccctggggccagggaaccctggtcactgtctcctca

SEQ ID NO: 290 - protein, 124 aa, synthetic construct
  REGION 1..124: note = synthetic
EVQLVESGGGLVQPGRSLRLSCAASGFTIDDSAMHWVRQTPGKGLEWVSGISWKSGSIGYADSVRGRFTISRDNAKNSLYLQMNSLRVEDTALYYCVKDIRGNWNYGGNWFDPWGQGTLVTVSS

SEQ ID NO: 291 - DNA, 24 bp, synthetic construct
  misc_feature 1..24: note = synthetic
  source 1..24: mol_type = other DNA
ggattcaccattgatgatagtgcc

SEQ ID NO: 292 - protein, 8 aa, synthetic construct
  REGION 1..8: note = synthetic
GFTIDDSA

SEQ ID NO: 293 - DNA, 24 bp, synthetic construct
  misc_feature 1..24: note = synthetic
  source 1..24: mol_type = other DNA
attagttggaaaagtggtagcata

SEQ ID NO: 294 - protein, 8 aa, synthetic construct
  REGION 1..8: note = synthetic
ISWKSGSI

SEQ ID NO: 295 - DNA, 51 bp, synthetic construct
  misc_feature 1..51: note = synthetic
  source 1..51: mol_type = other DNA
gtaaaagatataaggggcaactggaactacgggggaaactggttcgacccc

SEQ ID NO: 296 - protein, 17 aa, synthetic construct
  REGION 1..17: note = synthetic
VKDIRGNWNYGGNWFDP

SEQ ID NO: 297 - DNA, 345 bp, synthetic construct
  misc_feature 1..345: note = synthetic
  source 1..345: mol_type = other DNA
gaggtgcagctggtggagtctggaggaggcttggtccagcctggggggtccctgagactctcatgtgaagcctctgggttcaccgtcggtgtcaaccacatgaactgggtccgccaggctccagggaagggtctggagtgggtctcagttattttcagtagtggtaggacattctacggagactacgtgaaggggcgattaaccatcttcagacaaacctcccagaacacggtgtatcttcaaatgaatagcctgagaagtgaggacacggccatatattactgtgcgagagggattggcggtttggacatctggggccgagggacaatggtcaccgtctcttca

SEQ ID NO: 298 - protein, 115 aa, synthetic construct
  REGION 1..115: note = synthetic
EVQLVESGGGLVQPGGSLRLSCEASGFTVGVNHMNWVRQAPGKGLEWVSVIFSSGRTFYGDYVKGRLTIFRQTSQNTVYLQMNSLRSEDTAIYYCARGIGGLDIWGRGTMVTVSS

SEQ ID NO: 299 - DNA, 24 bp, synthetic construct
  misc_feature 1..24: note = synthetic
  source 1..24: mol_type = other DNA
gggttcaccgtcggtgtcaaccac

SEQ ID NO: 300 - protein, 8 aa, synthetic construct
  REGION 1..8: note = synthetic
GFTVGVNH

SEQ ID NO: 301 - DNA, 21 bp, synthetic construct
  misc_feature 1..21: note = synthetic
  source 1..21: mol_type = other DNA
attttcagtagtggtaggaca

SEQ ID NO: 302 - protein, 7 aa, synthetic construct
  REGION 1..7: note = synthetic
IFSSGRT

SEQ ID NO: 303 - DNA, 27 bp, synthetic construct
  misc_feature 1..27: note = synthetic
  source 1..27: mol_type = other DNA
gcgagagggattggcggtttggacatc

SEQ ID NO: 304 - protein, 9 aa, synthetic construct
  REGION 1..9: note = synthetic
ARGIGGLDI

SEQ ID NO: 305 - DNA, 369 bp, synthetic construct
  misc_feature 1..369: note = synthetic
  source 1..369: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctca

SEQ ID NO: 306 - protein, 123 aa, synthetic construct
  REGION 1..123: note = synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSS

SEQ ID NO: 307 - DNA, 24 bp, synthetic construct
  misc_feature 1..24: note = synthetic
  source 1..24: mol_type = other DNA
ggattcacctttgatgattatgcc

SEQ ID NO: 308 - protein, 8 aa, synthetic construct
  REGION 1..8: note = synthetic
GFTFDDYA

SEQ ID NO: 309 - DNA, 24 bp, synthetic construct
  misc_feature 1..24: note = synthetic
  source 1..24: mol_type = other DNA
attagttggactggtggtactata

SEQ ID NO: 310 - protein, 8 aa, synthetic construct
  REGION 1..8: note = synthetic
ISWTGGTI

SEQ ID NO: 311 - DNA, 48 bp, synthetic construct
  misc_feature 1..48: note = synthetic
  source 1..48: mol_type = other DNA
acaagagatatccgggggaactggaagtacggaggctggttcgacccc

SEQ ID NO: 312 - protein, 16 aa, synthetic construct
  REGION 1..16: note = synthetic
TRDIRGNWKYGGWFDP

SEQ ID NO: 313 - DNA, 360 bp, synthetic construct
  misc_feature 1..360: note = synthetic
  source 1..360: mol_type = other DNA
caggtgcagctggtgcagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctca

SEQ ID NO: 314 - protein, 120 aa, synthetic construct
  REGION 1..120: note = synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSS

SEQ ID NO: 315 - DNA, 24 bp, synthetic construct
  misc_feature 1..24: note = synthetic
  source 1..24: mol_type = other DNA
ggatacaccttcaccgcctactat

SEQ ID NO: 316 - protein, 8 aa, synthetic construct
  REGION 1..8: note = synthetic
GYTFTAYY

SEQ ID NO: 317 - DNA, 24 bp, synthetic construct
  misc_feature 1..24: note = synthetic
  source 1..24: mol_type = other DNA
atcagccctaacagtggtttcaca

SEQ ID NO: 318 - protein, 8 aa, synthetic construct
  REGION 1..8: note = synthetic
ISPNSGFT

SEQ ID NO: 319 - DNA, 39 bp, synthetic construct
  misc_feature 1..39: note = synthetic
  source 1..39: mol_type = other DNA
gcgcgagagggttctactcaccacaattctttcgacccc

SEQ ID NO: 320 - protein, 13 aa, synthetic construct
  REGION 1..13: note = synthetic
AREGSTHHNSFDP

SEQ ID NO: 321 - DNA, 342 bp, synthetic construct
  misc_feature 1..342: note = synthetic
  source 1..342: mol_type = other DNA
gaggtgcagctggtggagtctggaggaggcttggtccaaccgggggggtccctgaggctctcctgtgcagcctctgggttcaccgtcggtactaacttcatgaattgggtccgccaggctccagggaaggggctggagtgggtctcagcgatttatagcggtggtaccgctaactacgcagactccgtgaagggccgattcaccatttccagagacacttccaggaacacgctgtatcttcaaatgaacagcctgagaactgaggacacggccgtttattattgtgcgagaggggggggtatggacgtctggggccaagggaccacggtcaccgtctcctca

SEQ ID NO: 322 - protein, 114 aa, synthetic construct
  REGION 1..114: note = synthetic
EVQLVESGGGLVQPGGSLRLSCAASGFTVGTNFMNWVRQAPGKGLEWVSAIYSGGTANYADSVKGRFTISRDTSRNTLYLQMNSLRTEDTAVYYCARGGGMDVWGQGTTVTVSS

SEQ ID NO: 323 - DNA, 24 bp, synthetic construct
  misc_feature 1..24: note = synthetic
  source 1..24: mol_type = other DNA
gggttcaccgtcggtactaacttc

SEQ ID NO: 324 - protein, 8 aa, synthetic construct
  REGION 1..8: note = synthetic
GFTVGTNF

SEQ ID NO: 325 - DNA, 21 bp, synthetic construct
  misc_feature 1..21: note = synthetic
  source 1..21: mol_type = other DNA
atttatagcggtggtaccgct

SEQ ID NO: 326 - protein, 7 aa, synthetic construct
  REGION 1..7: note = synthetic
IYSGGTA

SEQ ID NO: 327 - DNA, 24 bp, synthetic construct
  misc_feature 1..24: note = synthetic
  source 1..24: mol_type = other DNA
gcgagaggggggggtatggacgtc

SEQ ID NO: 328 - protein, 8 aa, synthetic construct
  REGION 1..8: note = synthetic
ARGGGMDV

SEQ ID NO: 329 - DNA, 354 bp, synthetic construct
  misc_feature 1..354: note = synthetic
  source 1..354: mol_type = other DNA
caggtccagctggtgcagtctggggctgaggtgaagaagcctgggtcctcggtgaaggtctcctgcaaggcttctggaggcaccttcaacacctatgttctcagctgggtgcgacaggcccctggacaagggcttgagtggatgggagagatcatccctatcttaggtgcagcaaactacgcacagaacttccagggcagagtcacttttaccacggacgaatccacgaatacagcctacatggacctgagcagcctaagatctgaggacacggccgtgtattactgtgcgagagatcggacctccggggggttcgacccctggggccagggaaccctggtcactgtctcctca

SEQ ID NO: 330 - protein, 118 aa, synthetic construct
  REGION 1..118: note = synthetic
QVQLVQSGAEVKKPGSSVKVSCKASGGTFNTYVLSWVRQAPGQGLEWMGEIIPILGAANYAQNFQGRVTFTTDESTNTAYMDLSSLRSEDTAVYYCARDRTSGGFDPWGQGTLVTVSS

SEQ ID NO: 331 - DNA, 24 bp, synthetic construct
  misc_feature 1..24: note = synthetic
  source 1..24: mol_type = other DNA
ggaggcaccttcaacacctatgtt

SEQ ID NO: 332 - protein, 8 aa, synthetic construct
  REGION 1..8: note = synthetic
GGTFNTYV

SEQ ID NO: 333 - DNA, 24 bp, synthetic construct
  misc_feature 1..24: note = synthetic
  source 1..24: mol_type = other DNA
atcatccctatcttaggtgcagca

SEQ ID NO: 334 - protein, 8 aa, synthetic construct
  REGION 1..8: note = synthetic
IIPILGAA

SEQ ID NO: 335 - DNA, 33 bp, synthetic construct
  misc_feature 1..33: note = synthetic
  source 1..33: mol_type = other DNA
gcgagagatcggacctccggggggttcgacccc

SEQ ID NO: 336 - protein, 11 aa, synthetic construct
  REGION 1..11: note = synthetic
ARDRTSGGFDP

SEQ ID NO: 337 - DNA, 357 bp, synthetic construct
  misc_feature 1..357: note = synthetic
  source 1..357: mol_type = other DNA
caggttcagctggtgcagtctggagctgaggtggagaagcctggggcctcagtgaaggtctcctgcaaggcttctggttacatctttacccactatggtatcagctgggtgcgacaggcccctggacaaggacttgagtgggtgggctggatcagcccttacaatggttacacagactatgcacagaaactccagggcagagtcaccttgaccacagacacatccacgaccacagcctacatggagctgaggaacctgagatctgacgacacggccatgtattactgttcgagagggaggggcccttactggtccttcgatctctggggccgtggcaccctggtcaccgtctcctca

SEQ ID NO: 338 - protein, 119 aa, synthetic construct
  REGION 1..119: note = synthetic
QVQLVQSGAEVEKPGASVKVSCKASGYIFTHYGISWVRQAPGQGLEWVGWISPYNGYTDYAQKLQGRVTLTTDTSTTTAYMELRNLRSDDTAMYYCSRGRGPYWSFDLWGRGTLVTVSS

SEQ ID NO: 339 - DNA, 24 bp, synthetic construct
  misc_feature 1..24: note = synthetic
  source 1..24: mol_type = other DNA
ggttacatctttacccactatggt

SEQ ID NO: 340 - protein, 8 aa, synthetic construct
  REGION 1..8: note = synthetic
GYIFTHYG

SEQ ID NO: 341 - DNA, 24 bp, synthetic construct
  misc_feature 1..24: note = synthetic
  source 1..24: mol_type = other DNA
atcagcccttacaatggttacaca

SEQ ID NO: 342 - protein, 8 aa, synthetic construct
  REGION 1..8: note = synthetic
ISPYNGYT

SEQ ID NO: 343 - DNA, 36 bp, synthetic construct
  misc_feature 1..36: note = synthetic
  source 1..36: mol_type = other DNA
tcgagagggaggggcccttactggtccttcgatctc

SEQ ID NO: 344 - protein, 12 aa, synthetic construct
  REGION 1..12: note = synthetic
SRGRGPYWSFDL